(19) United States
(12) Patent Application Publication
(10) Pub. No.: US 2023/0331767 A1
(43) Pub. Date: Oct. 19, 2023

(54) TARGETED STEROID COMPOUNDS

(71) Applicant: Purdue Research Foundation, West Lafayette, IN (US)

(72) Inventors: Spencer S. Gardeen, Bloomington, MN (US); Philip Stewart Low, West Lafayette, IN (US)

(21) Appl. No.: 17/758,492

(22) PCT Filed: Jan. 7, 2021
(86) PCT No.: PCT/US2021/012532

§ 371 Date: Jul. 7, 2022

Related U.S. Application Data
(60) Provisional application No. 62/958,102, filed on Jan. 7, 2020.
(60) Provisional application No. 63/030,020, filed on May 26, 2020.

(51) Int. Cl. C07J 43/00; C07H 15/26; C07J 51/00; A61P 1/04

(52) U.S. Cl. CPC C07J 43/003; A61P 1/04; C07H 15/26; C07J 51/00

(57) ABSTRACT

A compound of the formula (I): G¹-L-G², or a pharmaceutically acceptable salt, polymorph, prodrug, solvate or clathrate thereof, wherein G¹ is a folate radical, an antifolate radical, or a folate analog radical; L is a linker; and G² is a radical of a steroid; compositions comprising such compounds; and the use of such compounds and compositions to treat, for example, inflammation associated with a disease or disorder.

### CROSS REFERENCE TO RELATED APPLICATIONS

This application claims priority to U.S. Provisional Pat. Application No. 62/958,102, which was filed on Jan. 7, 2020, and U.S. Provisional Pat. Application No. 63/030,020, which was filed on May 26, 2020, and which are hereby incorporated by reference in their entirety.

### BACKGROUND

Macrophages are a diverse group of white blood cells known for eliminating pathogens through phagocytosis. In the past, macrophages were classified by the organ in which they were found: Kupffer cells in the liver, Langerhans cells in the skin, microglia in the brain and spinal cord, and osteoclasts in the bone.

The current taxonomy for macrophages has shifted away from organ-specific macrophages to M1 and M2 macrophages. This classification is based upon macrophage polarization rather than macrophage location.

M1 macrophages are classically activated, typically by IFN-γ or lipopolysaccharide (LPS), and produce proinflammatory cytokines, phagocytize microbes, and initiate an immune response. M1 macrophages produce nitric oxide (NO) or reactive oxygen intermediates (ROI) to protect against bacteria and viruses.

M2 macrophages are alternatively activated by exposure to certain cytokines such as IL-4, IL-10, or IL-13. M2 macrophages will produce either polyamines to induce proliferation or proline to induce collagen production. These macrophages are associated with wound healing and tissue repair.

### SUMMARY

The disclosure relates to the design, synthesis, and testing of a series of compounds. Accordingly, the disclosure provides compounds comprising folate or related compounds as a ligand linked to a steroid via a linker. The linker can be any suitable linker, such as a hydrophilic linker. The linker can comprise one or more amino acids, a polyethylene glycol (PEG) monomer, a PEG oligomer, a PEG polymer, or a combination of any of the foregoing. The linker can comprise an oligomer of peptidoglycans, glycans, anions, or a combination of any of the foregoing. The compounds polarize macrophages from pro-inflammatory (M1) to anti-inflammatory (M2). In alternative embodiments, the ligand is a folate analog or an antifolate. The steroid can be any suitable steroid, such as dexamethasone, betamethasone or betamethasone 17-valerate. The steroid can be releasable, such as via reduction, oxidation, or hydrolysis, or non-releasable. The steroid can release via a self-immolative moiety.

Provided in some embodiments herein are compounds of the formula (I):

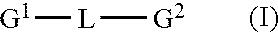

wherein:
G¹ is a folate radical, an antifolate radical, or a folate analog radical;
L is a linker; and
G² is a radical of a steroid;
or a pharmaceutically acceptable salt, polymorph, prodrug, solvate or clathrate thereof.

Disclosed herein, in certain embodiments, are compounds of the formula (I):

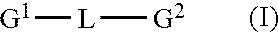

wherein:
G¹ is a folate radical, an antifolate radical, or a folate analog radical;
L is a linker; and
G² is a radical of a steroid;
or a pharmaceutically acceptable salt, polymorph, prodrug, solvate or clathrate thereof. In some embodiments, the folate radical has the formula:

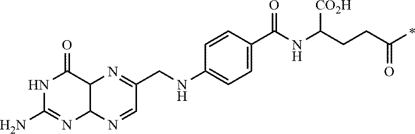 [SMILES: *C(=O)CCC(NC(=O)c1ccc(NCC2=NC3C(=O)NC(N)=NC3N=C2)cc1)C(=O)O]

wherein the asterisk denotes the point of attachment of the carbonyl carbon to the linker L. In some embodiments, G¹ is a pteroyl-amino acid radical where the amino acid is selected from the group consisting of aspartic acid, lysine, tyrosine, cysteine, threonine, serine, histidine, arginine, and an unnatural amino acid with a derivatizable moiety in the side chain. In some embodiments, G¹ is an antifolate radical or a folate analog radical comprising an amino acid selected from the group consisting of aspartic acid, lysine, tyrosine, cysteine, threonine, serine, histidine, and arginine. In some embodiments, G¹ is a radical of an antifolate of any one of formulas in Table 4 as described herein. In some embodiments, the steroid polarizes macrophages from pro-inflammatory (M1) to anti-inflammatory (M2). In some embodiments, G² is a radical of a steroid selected from the group consisting of betamethasone, cortisone, cortivazol, difluprednate, hydrocortisone, prednisolone, methylprednisolone, prednisone, dexamethasone, hydrocortisone-17-valerate, budesonide, flumethazone, fluticasone proprionate, fluorocortisone, fludrocortisone, paramethasone, eplerenone, and an ester of any of the foregoing. In some embodiments, G² is a radical of dexamethasone. In some embodiments, G² is a radical of prednisone. In some embodiments, G² is a radical of prednisolone. In some embodiments, G² is a radical of methylprednisolone. In some embodiments, G² is a radical of budesonide. In some embodiments, G² is a radical of triamcinolone. In some embodiments, G² is a radical of betamethasone. In some embodiments, the linker is releasable. In some embodiments, the linker is non-releasable. In some embodiments, the linker comprises one or more of an amino acid, an alkyl chain, a polyethylene glycol (PEG) monomer, a PEG oligomer, a PEG polymer, or a combination of any of the foregoing. In some embodiments, the linker increases the water solubility of the compound. In some embodiments, the linker comprises an oligomer of peptidoglycans, glycans, anions, or a combination of any of the foregoing. In some embodiments, the linker comprises at least one 2,3-diaminopropionic acid group, at least one glutamic acid group, and at least one cysteine group. In some embodiments, the linker comprises a repeating unit of the formula:

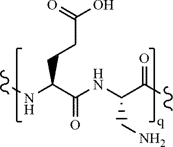

wherein q is an integer from 1 to 10. In some embodiments, the linker comprises the formula:

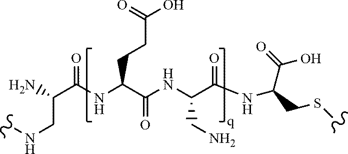

wherein q is an integer from 1 to 10. In some embodiments, the linker comprises the formula:

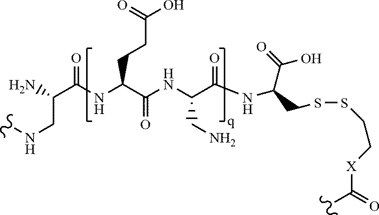

wherein X can be O, NH, NR, or S, and q is an integer from 1 to 10, wherein R is C₁₋₆ alkyl. In some embodiments, the linker comprises the formula:

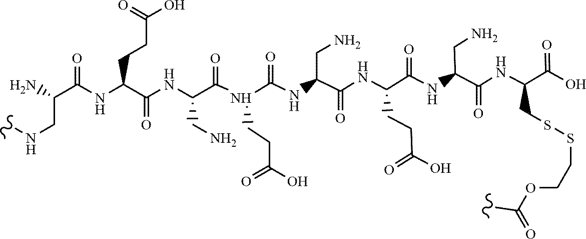

In some embodiments, the linker is a bivalent linker. In some embodiments, the linker is polyvalent and has multiple attachment points for one or more additional chemical groups. In some embodiments, the additional chemical groups comprise one or more additional G¹ groups. In some embodiments, the additional chemical groups comprise one or more binding ligands that are not G¹ groups. In some embodiments, the linker comprises a PEG oligomer with 2-16 PEG units. In some embodiments, the linker comprises a PEG oligomer with 12 PEG units. In some embodiments, the linker comprises an albumin ligand. In some embodiments, the albumin ligand comprises

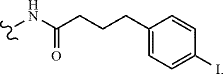

In some embodiments, the linker comprises a dimethylcysteine group. In some embodiments, the dimethylcysteine group is linked to a succinimide to form:

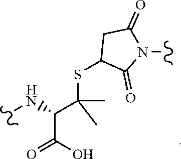

In some embodiments, the linker comprises a phosphate or pyrophosphate group. In some embodiments, the linker comprises a cathepsin B cleavable group. In some embodiments, the cathepsin B cleavable group is valine-citrulline. In some embodiments, the linker comprises a carbamate moiety. In some embodiments, the linker comprises a β-glucuronide. In some embodiments, the linker comprises an ester, phosphate, oxime, acetal, pyrophosphate, polyphosphate, disulfide, sulfate, hydrazide, imine, carbonate, carbamate or enzyme-cleavable amino acid sequence. In some embodiments, the linker comprises a self-immolative moiety. In some embodiments, the linker comprises a self-immolative disulfide and or sterically protected disulfide bond. In some embodiments, the linker comprises a self-immolative cathepsin-cleavable amino acid sequence. In some embodiments, the linker comprises a self-immolative furin-cleavable amino acid sequence. In some embodiments, the linker comprises a self-immolative β-glucuronidase-cleavable moiety. In some embodiments, the linker comprises a self-immolative phosphatase-cleavable moiety. In some embodiments, the linker comprises a self-immolative sulfatase-cleavable moiety. In some embodiments, the compound has the formula:

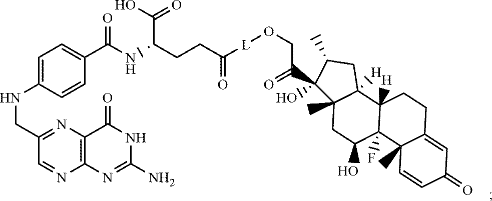

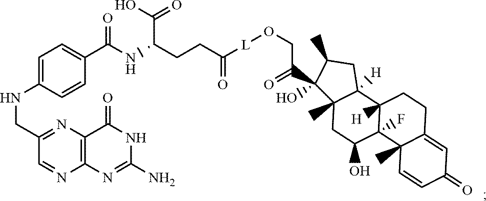

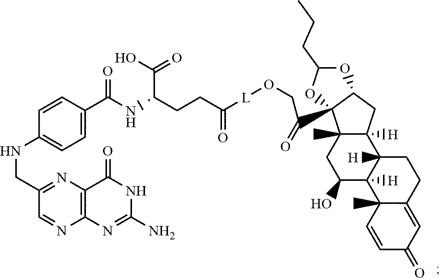

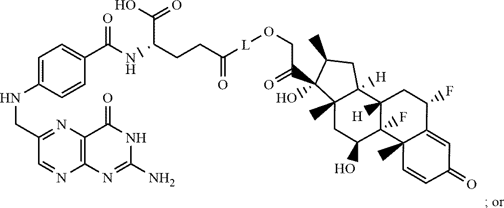

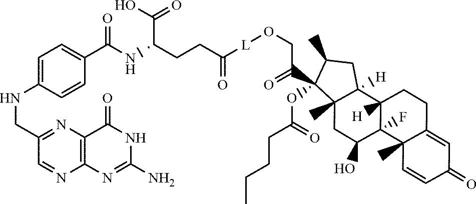

or a pharmaceutically acceptable salt, polymorph, prodrug, solvate or clathrate thereof. In some embodiments, the compound has the formula: 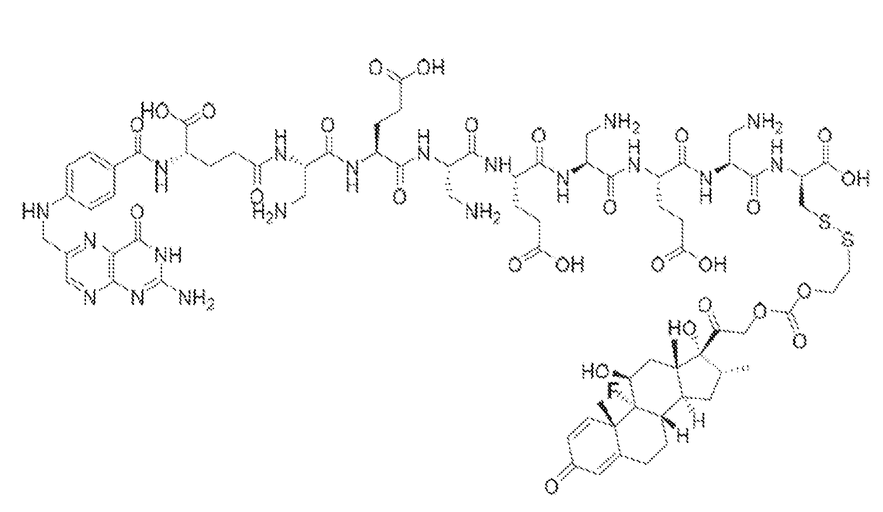 or a pharmaceutically acceptable salt, polymorph, prodrug, solvate or clathrate thereof. In some embodiments, the compound has the formula: 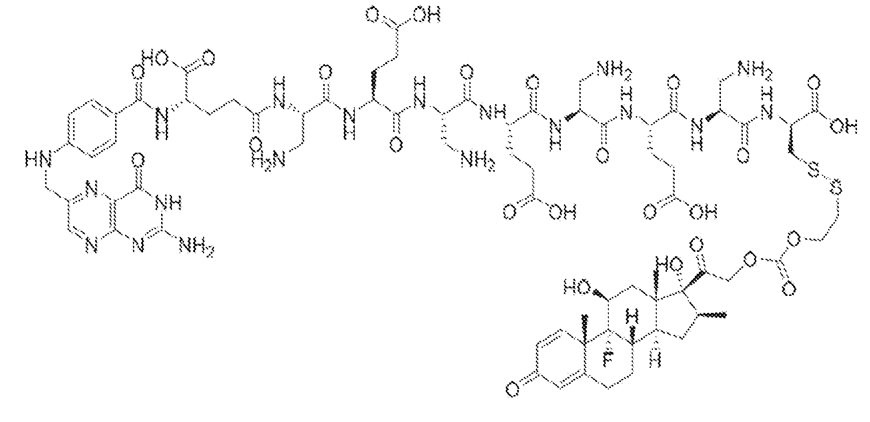 or a pharmaceutically acceptable salt, polymorph, prodrug, solvate or clathrate thereof. In some embodiments, the compound has the formula: 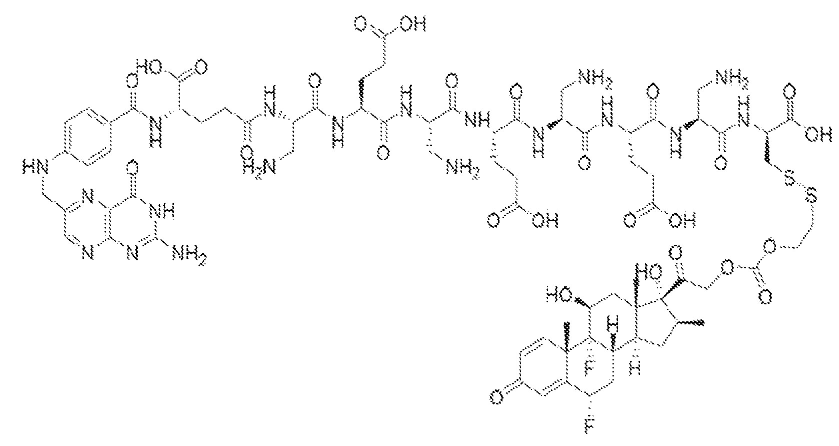 or a pharmaceutically acceptable salt, polymorph, prodrug, solvate or clathrate thereof. In some embodiments, the compound has the formula: 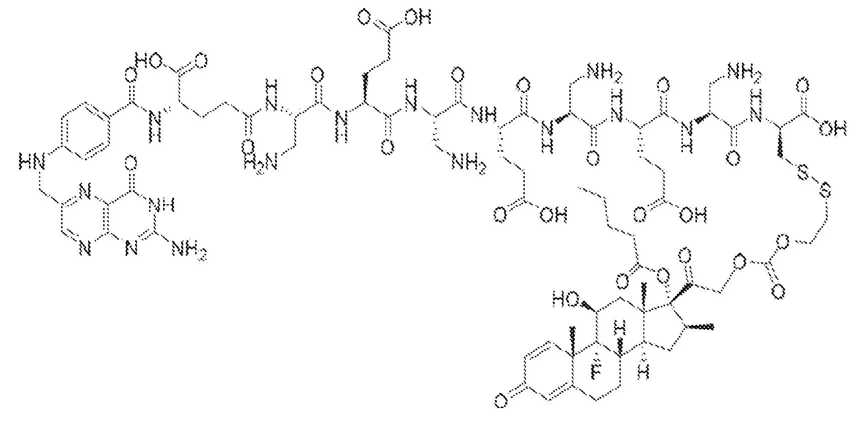 [SMILES: *C(=O)CCC(NC(=O)c1ccc(NCC2=NC3C(=O)NC(N)=NC3N=C2)cc1)C(=O)O] or a pharmaceutically acceptable salt, polymorph, prodrug, solvate or clathrate thereof. In some embodiments, the compound has the formula: 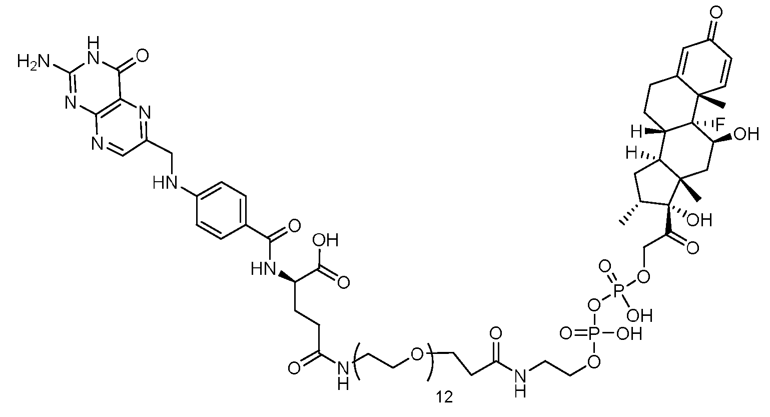 or a pharmaceutically acceptable salt, polymorph, prodrug, solvate or clathrate thereof. In some embodiments, the compound has the formula: 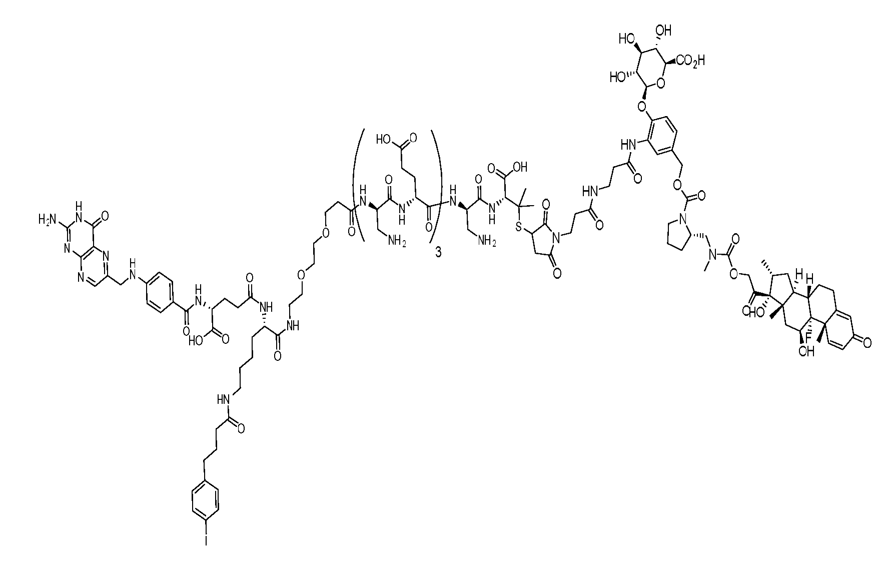 [SMILES: *C(=O)CCC(NC(=O)c1ccc(NCC2=NC3C(=O)NC(N)=NC3N=C2)cc1)C(=O)O] or a pharmaceutically acceptable salt, polymorph, prodrug, solvate or clathrate thereof. In some embodiments, the compound has the formula: 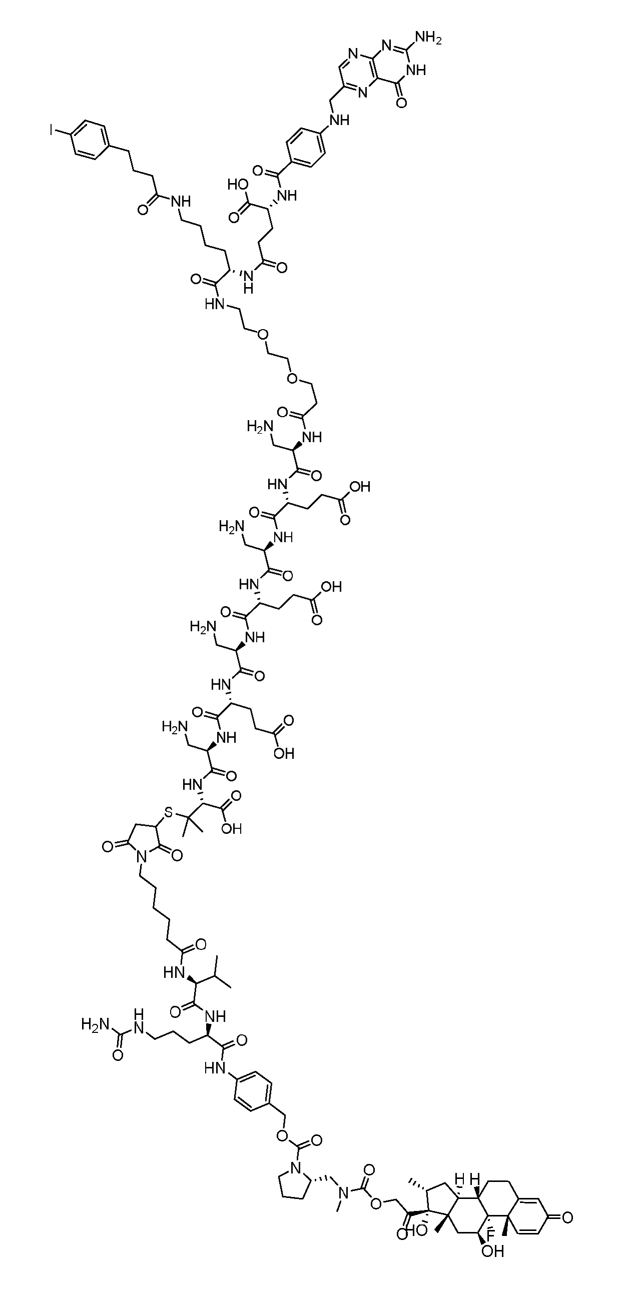 or a pharmaceutically acceptable salt, polymorph, prodrug, solvate or clathrate thereof. In some embodiments, the compound is selected from the compounds in the specification.

Disclosed herein, in certain embodiments, are pharmaceutical compositions, comprising: (a) a compound disclosed herein; and (b) a pharmaceutically acceptable excipient. In some embodiments, the pharmaceutically acceptable excipient is part of a nanoparticle, a liposomal formulation or an exosomal formulation.

Disclosed herein, in certain embodiments, are methods of shifting macrophages from M1 to M2 in a subject in need thereof, the method comprising: administering to the subject a therapeutically effective amount of a compound disclosed herein or a pharmaceutical composition disclosed herein.

Disclosed herein, in certain embodiments, are methods for treating an inflammatory disease or disorder (“disease” and “disorder” can be used interchangeably) in a subject in need thereof, the method comprising administering to the subject a therapeutically effective amount of a compound disclosed herein or a pharmaceutical composition disclosed herein. In some embodiments, G² is a radical of dexamethasone. In some embodiments, G² is a radical of prednisone. In some embodiments, G² is a radical of prednisolone. In some embodiments, G² is a radical of methylprednisolone. In some embodiments, G² is a radical of budesonide. In some embodiments, G² is a radical of triamcinolone. In some embodiments, G² is a radical of betamethasone. In some embodiments, the inflammatory disorder is Crohn’s disease, lupus, inflammatory bowel disease (IBS), Addison’s disease, Grave’s disease, Sjogren’s syndrome, celiac disease, Hashimoto’s thyroiditis, myasthenia gravis, autoimmune vasculitis, reactive arthritis, psoriatic arthritis, pernicious anemia, ulcerative colitis, rheumatoid arthritis, type 1 diabetes, multiple sclerosis, or fibrotic disease, graft vs. host disease (GVHD), fatty liver disease, asthma, osteoporosis, sarcoidosis, ischemia-reperfusion injury, prosthesis osteolysis, glomerulonephritis, scleroderma, psoriasis, with autoimmune myocarditis, spinal cord injury, central nervous system, viral infection, influenza, coronavirus infection, cytokine storm syndrome, bone damage, inflammatory brain disease, atherosclerosis, cancer, or a tumor. In some embodiments, the disorder is treated with reduced adverse effects relative to when the steroid is administered without linkage to the folate ligand.

Disclosed herein, in certain embodiments, are methods of treating an autoimmune disease in a subject in need thereof, the method comprising administering to the subject a therapeutically effective amount of a compound disclosed herein or a pharmaceutical composition disclosed herein. In some embodiments, G² is a radical of dexamethasone. In some embodiments, G² is a radical of prednisone. In some embodiments, G² is a radical of prednisolone. In some embodiments, G² is a radical of methylprednisolone. In some embodiments, G² is a radical of budesonide. In some embodiments, G² is a radical of triamcinolone. In some embodiments, G² is a radical of betamethasone.

Disclosed herein, in certain embodiments, are methods for treating inflammation in a subject in need thereof, the method comprising administering to the subject a therapeutically effective amount of a compound disclosed herein or a pharmaceutical composition disclosed herein. In some embodiments, the inflammation is treated with substantially reduced side effects relative to when the steroid is administered without linkage to the folate ligand. In some embodiments, the inflammation is associated with an autoimmune disease. In some embodiments, G² is a radical of a steroid selected from the group consisting of dexamethasone, betamethasone, budesonide, fludrocortisone, beclomethasone, fluticasone, mometasone, ciclesonide, cortisone, cortivazol, hydrocortisone, 21-hydroxypregnenolone, meprednisone, dexamethasone, triamcinolone, betamethasone, prednisone, prednisolone, and methylprednisolone. In some embodiments, G² is a radical of dexamethasone. In some embodiments, G² is a radical of prednisone. In some embodiments, G² is a radical of prednisolone. In some embodiments, G² is a radical of methylprednisolone. In some embodiments, G² is a radical of budesonide. In some embodiments, G² is a radical of triamcinolone. In some embodiments, G² is a radical of betamethasone. In some embodiments, the inflammation is associated with Crohn’s disease, lupus, inflammatory bowel disease (IBS), Addison’s disease, Grave’s disease, Sjogren’s syndrome, celiac disease, Hashimoto’s thyroiditis, myasthenia gravis, autoimmune vasculitis, reactive arthritis, psoriatic arthritis, pernicious anemia, ulcerative colitis, rheumatoid arthritis, type 1 diabetes, multiple sclerosis, or fibrotic disease, graft vs. host disease (GVHD), fatty liver disease, asthma, osteoporosis, sarcoidosis, ischemia-reperfusion injury, prosthesis osteolysis, glomerulonephritis, scleroderma, psoriasis, with autoimmune myocarditis, spinal cord injury, central nervous system, viral infection, influenza, coronavirus infection, cytokine storm syndrome, bone damage, inflammatory brain disease, atherosclerosis, cancer, or a tumor. In some embodiments, the inflammation is associated with Crohn’s disease, and G² is a radical of a steroid selected from the group consisting of dexamethasone, hydrocortisone, budesonide, betamethasone, prednisone, prednisolone, and methylprednisolone. In some embodiments, the inflammation is associated with lupus, and G² is a radical of a steroid selected from the group consisting of dexamethasone, hydrocortisone, betamethasone, budesonide, prednisone, prednisolone, and methylprednisolone. In some embodiments, the inflammation is associated with inflammatory bowel disease, and wherein G² is a radical of a steroid selected from the group consisting of dexamethasone, betamethasone, hydrocortisone, budesonide, prednisone, prednisolone, and methylprednisolone. In some embodiments, the inflammation is associated with Addison’s disease, and G² is a radical of a steroid selected from the group consisting of dexamethasone, betamethasone, hydrocortisone, fludrocortisone, prednisone, prednisolone, and methylprednisolone. In some embodiments, the inflammation is associated with Grave’s disease, and G² is a radical of a steroid selected from the group consisting of dexamethasone, betamethasone, budesonide, prednisone, prednisolone, and methylprednisolone. In some embodiments, the inflammation is associated with Sjogren’s syndrome, and G² is a radical of a steroid selected from the group consisting of dexamethasone, betamethasone, budesonide, prednisone, prednisolone, and methylprednisolone. In some embodiments, the inflammation is associated with celiac disease, and G² is a radical of a steroid selected from the group consisting of dexamethasone, betamethasone, budesonide, prednisone, prednisolone, and methylprednisolone. In some embodiments, the inflammation is associated with Hashimoto’s thyroiditis, and wherein G² is a radical of a steroid selected from the group consisting of dexamethasone, betamethasone, budesonide, prednisone, prednisolone, and methylprednisolone. In some embodiments, the inflammation is associated with myasthenia gravis, and G² is a radical of a steroid selected from the group consisting of dexamethasone, betamethasone, budesonide, prednisone, prednisolone, and methylprednisolone. In some embodiments, the inflammation is associated with autoimmune vasculitis, and G² is a radical of a steroid selected from the group consisting of dexamethasone, betamethasone, budesonide, prednisone, prednisolone, and methylprednisolone. In some embodiments, the inflammation is associated with reactive arthritis, and G² is a radical of a steroid selected from the group consisting of cortisone, hydrocortisone, dexamethasone, betamethasone, budesonide, prednisone, prednisolone, and methylprednisolone. In some embodiments, the inflammation is associated with psoriatic arthritis, and G² is a radical of a steroid selected from the group consisting of cortisone, hydrocortisone, dexamethasone, betamethasone, budesonide, prednisone, prednisolone, and methylprednisolone. In some embodiments, the inflammation is associated with pernicious anemia, and G² is a radical of a steroid selected from the group consisting of dexamethasone, betamethasone, prednisone, prednisolone, and methylprednisolone. In some embodiments, the inflammation is associated with ulcerative colitis, and G² is a radical of a steroid selected from the group consisting of cortisone, hydrocortisone, triamcinolone, beclomethasone, betamethasone, dexamethasone, budesonide, prednisone, prednisolone, and methylprednisolone. In some embodiments, the inflammation is associated with rheumatoid arthritis, and G² is a radical of a steroid selected from the group consisting of cortisone, hydrocortisone, triamcinolone, beclomethasone, betamethasone, dexamethasone, budesonide, prednisone, prednisolone, and methylprednisolone. In some embodiments, the inflammation is associated with type 1 diabetes, and G² is a radical of a steroid selected from the group consisting of dexamethasone, betamethasone, prednisone, prednisolone, and methylprednisolone. In some embodiments, the inflammation is associated with multiple sclerosis, and G² is a radical of a steroid selected from the group consisting of dexamethasone, betamethasone, prednisone, prednisolone, and methylprednisolone. In some embodiments, the inflammation is associated with asthma, and G² is a radical of a steroid selected from the group consisting of triamcinolone, fluticasone, budesonide, mometasone, beclomethasone, ciclesonide, dexamethasone, betamethasone, budesonide, prednisone, prednisolone, and methylprednisolone. In some embodiments, the inflammation is associated with osteoporosis, and G² is a radical of a steroid selected from the group consisting of dexamethasone, betamethasone, prednisone, prednisolone, and methylprednisolone. In some embodiments, the inflammation is associated with sarcoidosis, and G² is a radical of a steroid selected from the group consisting of dexamethasone, betamethasone, prednisone, prednisolone, and methylprednisolone. In some embodiments, the inflammation is associated with glomerulonephritis, and G² is a radical of a steroid selected from the group consisting of dexamethasone, betamethasone, budesonide, prednisone, prednisolone, and methylprednisolone. In some embodiments, the inflammation is associated with autoimmune myocarditis, and G² is a radical of a steroid selected from the group consisting of dexamethasone, betamethasone, budesonide, prednisone, prednisolone, and methylprednisolone. In some embodiments, the inflammation is associated with a fibrotic disease, and G² is a radical of a steroid selected from the group consisting of dexamethasone, betamethasone, budesonide, and prednisone. In some embodiments, the inflammation is associated with graft vs. host disease (GVHD), and G² is a radical of a steroid selected from the group consisting of dexamethasone, betamethasone, budesonide, prednisone, prednisolone, and methylprednisolone. In some embodiments, the inflammation is associated with fatty liver disease, and G² is a radical of a steroid selected from the group consisting of dexamethasone, betamethasone, budesonide, prednisone, prednisolone, and methylprednisolone. In some embodiments, the inflammation is associated with ischemia-reperfusion injury, and G² is a radical of a steroid selected from the group consisting of dexamethasone, betamethasone, budesonide, prednisone, prednisolone, and methylprednisolone. In some embodiments, the inflammation is associated with prosthesis osteolysis, and G² is a radical of a steroid selected from the group consisting of dexamethasone, betamethasone, budesonide, prednisone, prednisolone, and methylprednisolone. In some embodiments, the inflammation is associated with scleroderma, and G² is a radical of a steroid selected from the group consisting of dexamethasone, budesonide, betamethasone, triamcinolone, prednisone, prednisolone, and methylprednisolone. In some embodiments, the inflammation is associated with psoriasis, and G² is a radical of a steroid selected from the group consisting of budesonide, betamethasone, triamcinolone, prednisone, prednisolone, methylprednisolone, hydrocortisone, dexamethasone, hydrocortisone-17-valerate, diflorasone, meprednisone, halobetacol, tixocortol, amcinonide, desonide, fluocinolone acetonide, fluocinonide, halcinonide, beclomethasone, and halometasone. In some embodiments, the inflammation is associated with spinal cord injury, and G² is a radical of a steroid selected from the group consisting of dexamethasone, betamethasone, budesonide, prednisone, prednisolone, and methylprednisolone. In some embodiments, the inflammation is of the central nervous system, and G² is a radical of a steroid selected from the group consisting of dexamethasone, betamethasone, budesonide, prednisone, prednisolone, and methylprednisolone. In some embodiments, the inflammation is associated with a viral infection, and G² is a radical of a steroid selected from the group consisting of hydrocortisone, dexamethasone, betamethasone, budesonide, prednisone, prednisolone, and methylprednisolone. In some embodiments, the inflammation is associated with influenza, and G² is a radical of a steroid selected from the group consisting of hydrocortisone, dexamethasone, betamethasone, budesonide, prednisone, prednisolone, and methylprednisolone. In some embodiments, the inflammation is associated with SARS-CoV-2 (COVID-19), and G² is a radical of a steroid selected from the group consisting of hydrocortisone, dexamethasone, betamethasone, budesonide, and prednisone, prednisolone, and methylprednisolone. In some embodiments, the inflammation is associated with cytokine storm syndrome, and G² is a radical of a steroid selected from the group consisting of hydrocortisone, dexamethasone, betamethasone, budesonide, prednisone, prednisolone, and methylprednisolone. In some embodiments, the inflammation is associated with damage to bone, and G² is a radical of a steroid selected from the group consisting of cortisone, cortivazol, hydrocortisone, 21-hydroxypregnenolone, meprednisone, dexamethasone, triamcinolone, betamethasone, budesonide, prednisone, prednisolone, and methylprednisolone. In some embodiments, the inflammation is associated with inflammatory brain disease, and G² is a radical of a steroid selected from the group consisting of dexamethasone, betamethasone, budesonide, prednisone, prednisolone, and methylprednisolone. In some embodiments, the inflammation is associated with atherosclerosis, and G² is a radical of a steroid selected from the group consisting of fluticasone, budesonide, beclomethasone, ciclesonide, dexamethasone, betamethasone, prednisone, prednisolone, and methylprednisolone. In some embodiments, the inflammation is associated with a tu or, and wherein G² is a radical of a steroid selected from the group consisting of cortisone, hydrocortisone, clobetasol, dexamethasone, betamethasone, budesonide, meprednisone, prednisone, prednisolone, and methylprednisolone. In some embodiments, the inflammation is associated with cancer, and G² is a radical of a steroid selected from the group consisting of cortisone, hydrocortisone, clobetasol, dexamethasone, betamethasone, budesonide, meprednisone, prednisone, prednisolone, and methylprednisolone.

Disclosed herein, in certain embodiments, are methods for treating a disease or disorder that involves polarizing macrophages from M1 to M2 in a subject in need thereof, the method comprising administering to the subject a therapeutically effective amount of a compound disclosed herein or a pharmaceutical composition disclosed herein.

### BRIEF DESCRIPTION OF THE DRAWINGS

FIG. 1 exemplifies the chemical structure for compound 101 of the disclosure.

FIG. 2 exemplifies a reaction scheme for the activation of dexamethasone for linkage to a ligand comprising folate and a moiety that becomes a linker when reacted with the activated dexamethasone.

FIG. 3 exemplifies the chemical structure of a ligand comprising folate and a moiety that becomes a linker when reacted with the activated dexamethasone shown in FIG. 2.

FIG. 4 exemplifies a reaction scheme for the formation of compound 101, by reaction of the activated dexamethasone with the compound of FIG. 3.

FIG. 5 exemplifies structures and names of steroids described in the disclosure.

FIG. 6 is a plot of CD206 (a marker for M2 macrophages) expression vs. percent of maximum (Max) showing flow cytometry results of compound 101 in the absence and presence of the competitor folate-glucosamine.

FIG. 7 is a plot of CD206 (a marker for M2 macrophages) expression vs. percent of maximum (Max) showing flow cytometry results of compound 101 in the absence and presence of the competitor folate-glucosamine compared to untreated cells and cells treated with free dexamethasone.

FIG. 8 is a plot of CD86 (a marker for M1 macrophages) expression vs. percent of maximum (Max) showing flow cytometry results of compound 101 in the absence and presence of the competitor folate-glucosamine compared to untreated cells and cells treated with free dexamethasone.

FIG. 9 is a set of plots of flow cytometery results for F4/80 rodent colon cells in a peritonitis model, and includes results for cells treated with compound 106, untreated, and vehicle.

FIG. 10 is a bar graph of flow cytometery results for F4/80 rodent colon cells in a peritonitis model, and includes results for cells treated with compound 106, untreated, and vehicle.

FIG. 11 is a set of plots of flow cytometery results for F4/80, CD4, Ly6G, and CD8 rodent colon cells in a peritonitis model, and includes results for cells treated with compound 107, and untreated.

FIG. 12 is a bar graph of flow cytometery results for F4/80, CD4, Ly6G, and CD8 rodent colon cells in a peritonitis model, and includes results for cells treated with compound 107, and untreated.

FIG. 13 is a set of plots of flow cytometery results for F4/80, CD4, Ly6G, and CD8 rodent colon cells in a peritonitis model, and includes results for cells treated with compound 108, and untreated.

FIG. 14 is a bar graph of flow cytometery results for F4/80, CD4, Ly6G, and CD8 rodent colon cells in a peritonitis model, and includes results for cells treated with compound 108, and untreated.

FIG. 15 is a set of two images of mouse colons, one from an untreated mouse and one from a mouse treated with compound 107.

FIG. 16 is a set of LC-MS traces for compound 101.

FIG. 17 is a set of LC-MS traces for compound 106.

FIG. 18 is a set of LC-MS traces for compound 107.

FIG. 19 is a set of LC-MS traces for compound 125.

FIG. 20 is a set of LC-MS traces for compound 108.

FIG. 21 is a set of LC-MS traces for compound 124.

FIG. 22 is a set of LC-MS traces for compound 126.

FIG. 23 is a set of LC-MS traces for compound 127.

It is to be understood that the drawings are not intended to limit the scope of the present teachings in any way.

### DETAILED DESCRIPTION

Folate receptor beta is expressed on the surface of activated macrophages that are present at sites of inflammation. These macrophages have been shown to be crucial in maintaining levels of pro-inflammatory signals in a variety of diseases.

Steroids work by decreasing inflammation and reducing the activity of the immune system. Inflammation is a process in which the body’s white blood cells and chemicals can protect against infection and foreign substances such as bacteria and viruses. In certain diseases, however, the body’s defense system (immune system) doesn’t function properly. This might cause inflammation to work against the body’s tissues and cause damage. Signs of inflammation include redness, warmth, swelling, and pain.

Steroids reduce the production of chemicals that cause inflammation. This helps keep tissue damage as low as possible. Steroids also reduce the activity of the immune system by affecting the way white blood cells work.

There are associated side effects of steroid use depending on the dose, type of steroid and the length of treatment. To avoid these side effects, the following guidelines are helpful: use steroids only when necessary and, if possible, use local steroids for local problems; use the smallest dose needed to control the disease; and reduce the dose gradually as long as the disease remains under control.

The above side effects could be minimized by targeted delivery of steroid to the site of inflammation. For example, steroid could be delivered to activated macrophages by a compound that targets the cell-surface folate receptor beta. To date, the inventors have not seen evidence of the synthesis and efficacy of the use of folate as a ligand linked to a steroid in the literature. Therefore, there is an unmet need for such a compound.

### Definitions

Unless defined otherwise, all technical and scientific terms used herein have the same meaning as commonly understood by one of skill in the chemical and biological arts. Additionally, as used in this specification and the appended claims, the singular forms “a”, “an” and “the” include plural referents unless the content clearly dictates otherwise. Thus, for example, where a compound/composition is substituted with “an” alkyl or aryl, the compound/composition is optionally substituted with at least one alkyl and/or at least one aryl. Furthermore, unless specifically stated otherwise, the term “about” refers to a range of values plus or minus 10% for percentages and plus or minus 1.0 unit for unit values, for example, about 1.0 refers to a range of values from 0.9 to 1.1.

If a chemical group combines several other chemical groups defined herein, then each part of the combination is assumed to be defined as when it is separate, with allowances made to create valences for allowing attachment of the other groups. For example, “alkoxycycloalkylenecarbonyl” radical would be understood to be an alkoxy as defined herein bonded to a cycloalkylene as defined herein, and the cycloalkylene is in turn bonded to a carbonyl group, which is not defined herein but is generally understood to organic chemists, with an open valence on the carbonyl.

The term “radical” as used herein refers to a fragment of a molecule, wherein that fragment has an open valence for bond formation. A monovalent radical has one open valence such that it can form one bond with another chemical group. In some embodiments, a radical of a molecule (e.g., a radical of a steroid) as used herein is created by removal of one hydrogen atom from that molecule to create a monovalent radical with one open valence at the location where the hydrogen atom was removed. Where appropriate, a radical can be divalent, trivalent, etc., wherein two, three or more hydrogen atoms have been removed to create a radical which can bond to two, three, or more chemical groups. Where appropriate, a radical open valence may be created by removal of other than a hydrogen atom (e.g., a halogen atom), or by removal of two or more atoms (e.g., a hydroxyl group), as long as the atoms removed are a small fraction (20% or less of the atom count) of the total atoms in the molecule forming the radical. In some embodiments, a radical is formed from a folate, antifolate, or folate analog by removal of a hydroxyl group.

The term “substituted” or “substituent” as used herein refers to a group that can be or is substituted onto a molecule or onto another group (e.g., on an aryl or an alkyl group). Examples of substituents include, but are not limited to, a halogen (e.g., F, Cl, Br, and I), OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, -(CH₂)₀₋₂P(O)(OR)₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH2)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(=NH)N(R)₂,C(O)N(OR)R, or C(=NOR)R wherein each R can be, independently, hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or two R groups bonded to a nitrogen atom or to adjacent nitrogen atoms can together with the nitrogen atom or atoms form a heterocyclyl, which can be mono- or independently multi-substituted.

An “individual,” “subject” or “patient,” as used herein, is a mammal, preferably a human, but can also be an animal.

“Oxo” refers to the ═O radical.

“Alkyl” generally refers to a straight or branched hydrocarbon chain radical consisting solely of carbon and hydrogen atoms, such as having from one to fifteen carbon atoms (e.g., C₁-C₁₅ alkyl). Disclosures provided herein of an “alkyl” are intended to include independent recitations of a saturated “alkyl,” unless otherwise stated. In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

“Alkoxy” refers to a radical bonded through an oxygen atom of the formula -O-alkyl, where alkyl is an alkyl chain as defined above.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

“Aralkyl” or “aryl-alkyl” refers to a radical of the formula -R^{c}-aryl where R^{c} is an alkylene chain as defined above, for example, methylene, ethylene, and the like. The alkylene chain part of the aralkyl radical is optionally substituted as described above for an alkylene chain.

“Cycloalkyl” refers to a stable 3- to 18-membered non-aromatic ring radical that comprises only carbon atoms as ring atoms. Unless stated otherwise specifically in the specification, the cycloalkyl radical is a monocyclic, bicyclic, tricyclic or tetracyclic ring system, which optionally includes aromatic, fused, and/or bridged ring systems. Examples of such radicals include cyclopropyl, cyclohexyl, norbornyl, and adamantyl. “Cycloalkylene” as used herein specifically refers to a divalent cycloalkyl radical.

“Halo” or “halogen” refers to bromo, chloro, fluoro or iodo substituents.

“Haloalkyl” refers to an alkyl radical, as defined above, that is substituted by one or more halogen radicals, as defined above, for example, trifluoromethyl, difluoromethyl, fluoromethyl, 2,2,2-trifluoroethyl, 1-fluoromethyl-2-fluoroethyl, and the like.

The term “heteroalkyl” refers to an alkyl group as defined above in which one or more skeletal carbon atoms of the alkyl are substituted with a heteroatom (with the appropriate number of substituents or valencies - for example, —CH₂— may be replaced with —NH— or —O—). For example, each substituted carbon atom is independently substituted with a heteroatom, such as wherein the carbon is substituted with a nitrogen, oxygen, selenium, or other suitable heteroatom. In some instances, each substituted carbon atom is independently substituted for an oxygen, nitrogen (e.g. —NH—, -N(alkyl)-, or -N(aryl)- or having another substituent contemplated herein), or sulfur (e.g. —S—, —S(═O)—, or —S(═O)₂—). In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₁₈ heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁-C₄ heteroalkyl. In some embodiments, heteroalkyl includes alkoxy, alkoxyalkyl, alkylamino, alkylaminoalkyl, aminoalkyl, and heterocycloalkyl, heterocycloalkylalkyl, as defined herein.

“Heteroalkylene” refers to a divalent heteroalkyl group defined above which links one part of the molecule to another part of the molecule.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes aromatic, fused, and/or bridged ring systems. The heteroatoms in the heterocyclyl radical are optionally oxidized. The heterocyclyl radical is partially or fully saturated. Disclosures provided herein of an “heterocyclyl” are intended to include independent recitations of heterocyclyl comprising aromatic and non-aromatic ring structures, unless otherwise stated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, 1,3-benzodioxolyl, 1,4-benzodioxanyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, indolinyl, isoindolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, benzofuranyl, benzooxazolyl, benzo[d]thiazolyl, benzothiadiazolyl, benzo[b][1,4]dioxepinyl, benzo[b][1,4]oxazinyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl).

The term “heterocycloalkyl” as used herein refers to alkyl groups as defined herein in which a hydrogen or carbon bond of an alkyl group as defined herein is replaced with a bond to a heterocyclyl group as defined herein. Representative heterocycloalkyl groups include, but are not limited to, furan-2-yl methyl, furan-3-yl methyl, pyridine-3-yl methyl, tetrahydrofuran-2-yl methyl, and indol- 2-yl propyl. The term “heterocycloalkylalkyl” as used herein refers to a heterocycloalkyl group attached to an alkyl group, as defined herein.

The term “heteroarylalkyl” as used herein refers to alkyl groups as defined herein in which a hydrogen or carbon bond of an alkyl group is replaced with a bond to a heteroaryl group as defined herein.

The term “amine” as used herein refers to primary, secondary, and tertiary amines having, e.g., the formula N(group)₃⁺ wherein each group can independently be H or non-H, such as alkyl, aryl, and the like. Amines include but are not limited to R-NH₂, for example, alkylamines, arylamines, alkylarylamines; R₂NH wherein R is defined herein, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form -NH₂, -NHR, -NR₂, -NR₃⁺, wherein each R is defined herein, and protonated forms of each, except for -NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

An example of a “alkylamino” is -NH-alkyl and -N(alkyl)₂.

The compounds disclosed herein, in some embodiments, contain one or more asymmetric centers and thus give rise to enantiomers, diastereomers, and other stereoisomeric forms that are defined, in terms of absolute stereochemistry, as (R)- or (5)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para- isomers around a benzene ring.

As used herein, the term “linker” generally refers to a portion of a compound that forms a chemical bond with a G¹ (e.g., a binding ligand) and/or G² (e.g., a therapeutic agent). In particular, a “linker” can connect two or more functional parts of a molecule to form a compound provided herein. Illustratively, the linker may comprise atoms selected from C, N, O, S, Si, and P; C, N, O, S, and P; or C, N, O, and S. The linker may connect different functional capabilities of the compound, such as the folate ligand and the therapeutic agent. The linker may comprise a several linker groups, such as, for example, in the range from about 2 to about 100 atoms in the contiguous backbone. In some embodiments, the linker is a releasable linker. In some embodiments, the linker is a non-releasable linker.

A “pteroyl” radical, moiety, or group as used herein has the following structure:

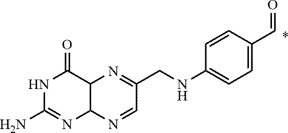

wherein the asterisk denotes the point of attachment of the carbonyl carbon to another chemical group, such as the linker L.

A “pteroyl-amino acid” radical, moiety, or group as used herein has the following structure:

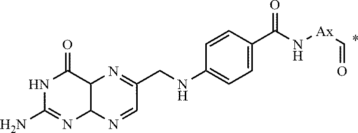

wherein the asterisk denotes the point of attachment of the carbonyl carbon to another chemical group, such as the linker L, and wherein H₂N-Ax-COOH is an amino acid.

“Reduced adverse events” in a subject, as used herein refers to a reduction of the prevalence and/or severity of adverse events occurring in a treated patient, wherein that reduction is significant with at least a 95% confidence level. “Adverse events” as used herein include serious events causing death, hospitalization and/or disability (temporary or permanent), as well as less serious events causing discomfort (such as skin rash, sleep problems, hair loss, headache) which is noticeable by the subject.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional nontoxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington’s Pharmaceutical Sciences, 21st ed., Lippincott Williams & Wilkins, 2006, e.g., Chapter 38, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound disclosed herein. Examples of prodrugs include, but are not limited to, derivatives and metabolites of a compound disclosed herein that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger’s Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

The term “polymorph” generally refers to crystalline materials that have the same chemical composition but different molecular packing. The term “crystalline salt” includes crystalline structures with the same chemical materials, but incorporating acid or base addition salts within the molecular packing of the crystalline structure.

The term “clathrate” generally refers to a compound in which molecules of one component (e.g., solvent) are physically trapped within the crystal structure of another.

The terms “non-releasable linker” or “non-cleavable linker” are used interchangeably. As used herein, they refer to a linker that cannot be cleaved under extracellular physiological conditions (e.g., a pH-labile, acid-labile, oxidatively-labile, or enzyme-labile bond). However, such a linker may include bonds that may be cleaved after entry into a cell.

The term “releasable linker” as used herein refers to a linker that includes at least one bond that can be cleaved under extracellular physiological conditions (e.g., a pH-labile, acid-labile, oxidatively-labile, or enzyme-labile bond). Releasable groups also include photochemically-cleavable groups. Examples of photochemically-cleavable groups include 2-(2-nitrophenyl)-ethan-2-ol groups and linkers containing o-nitrobenzyl, desyl, trans-o-cinnamoyl, m-nitrophenyl or benzylsulfonyl groups (see, for example, Dorman and Prestwich, Trends Biotech. 18:64-77 (2000); and Greene and Wuts, Protective Groups in Organic Synthesis, 2nd ed., John Wiley & Sons, New York (1991)).

The cleavable bond or bonds may be present in the interior of a cleavable linker and/or at one or both ends of a cleavable linker. It should be appreciated that such physiological conditions resulting in bond cleavage include standard chemical hydrolysis reactions that occur, for example, at physiological pH, or as a result of compartmentalization into a cellular organelle such as an endosome having a lower pH than cytosolic pH. Illustratively, the bivalent linkers described herein can undergo cleavage under other physiological or metabolic conditions, such as by the action of a glutathione mediated mechanism. It is appreciated that the lability of the cleavable bond may be adjusted by including functional groups or fragments within the bivalent linker L that are able to assist or facilitate such bond cleavage, also termed anchimeric assistance. The lability of the cleavable bond can also be adjusted by, for example, substitutional changes at or near the cleavable bond, such as including alpha branching adjacent to a cleavable disulfide bond, increasing the hydrophobicity of substituents on silicon in a moiety having a silicon-oxygen bond that may be hydrolyzed, homologating alkoxy groups that form part of a ketal or acetal that may be hydrolyzed, and the like. In addition, it is appreciated that additional functional groups or fragments may be included within the bivalent linker L that are able to assist or facilitate additional fragmentation of the compounds after bond breaking of the releasable linker, when present.

As used herein, the terms “subject,” “patient,” and “individual” are used interchangeably. None of the terms are intended to require the continuous supervision of a medical professional. The subject can be any mammal, for example a human.

The term “treating,” as used herein, encompasses therapeutic treatment (e.g., a subject with signs and symptoms of a disease state being treated) and/or prophylactic treatment. Prophylactic treatment encompass prevention and inhibition or delay of progression of a disease state.

The term “therapeutically effective amount” as used herein, refers to that amount of one or more compounds of the various embodiments described herein (e.g. a compound of the formula (I)) that elicits a biological or medicinal response in a tissue system, animal or human, that is being sought by a researcher, veterinarian, medical doctor or other clinician, which includes alleviation of the signs or symptoms of the disease or disorder being treated.

As used herein, an “antifolate” is a compound that binds to a folate receptor and antagonizes the biological actions of folic acid or one of its naturally occurring forms such as dihydrofolate, 5-methyltetrahydrofolate, or methylene tetrahydrofolates. Antifolates include inhibitors of dihydrofolate reductase, thymidylate synthase and other enzymes in the folate biosynthesis pathway. Antifolates include methotrexate, pemetrexed, proguanil, pyrimethamine, ralitrexed, pralatrexate, and trimethoprim. Antifolates further include compounds described in Table 4 herein.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section.

The term “about” as used herein can allow for a degree of variability in a value or range, for example, within 10%, within 5%, or within 1% of a stated value or of a stated limit of a range.

The terms and expressions, which have been employed, are used as terms of description and not of limitation. In this regard, where certain terms are defined under “Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Definitions,” are used in the “Detailed Description,” such use is solely for ease of reference and is not intended to limit any disclosure made in one section to that section only; rather, any disclosure made under one subheading is intended to constitute a disclosure under each and every other subheading.

### Compounds

In one aspect, this disclosure relates to the design, synthesis, and testing of a series of compounds. According, provided are compounds of the formula (I):

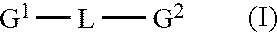

or a pharmaceutically acceptable salt, polymorph, prodrug, solvate or clathrate thereof, wherein:
G¹ is a folate radical, an antifolate radical, or a folate analog radical;
L is a linker; and
G² is a radical of a steroid.

In some embodiments, the folate radical is a group of the formula:

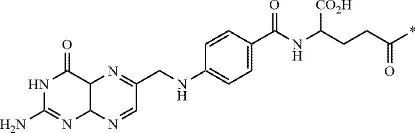

wherein the asterisk (*) denotes the point of attachment of the carbonyl carbon to the linker L. In alternative embodiments, G¹ can be a radical of an antifolate (e.g., a folate antagonist or a folic acid antagonist) or a folate analog radical. In some embodiments, G¹ is a radical of an antifolate of any one of formulas in Table 4 as described herein

In some embodiments, G¹ is a folate analog radical. In some embodiments, the folate analog is a pteroyl moiety or a pteroyl-amino acid moiety or a folate analog of any one of formulas V-X as described herein. In some embodiments, G¹ is a pteroyl-amino acid radical. In some embodiments, G¹ is a pteroyl-amino acid radical where the amino acid is selected from the group consisting of aspartic acid, lysine, tyrosine, cysteine, threonine, serine, histidine, arginine, and an unnatural amino acid with a derivatizable moiety in the side chain.

In some embodiments, G¹ is an antifolate radical or a folate analog radical comprising an amino acid selected from the group consisting of aspartic acid, lysine, tyrosine, cysteine, threonine, serine, histidine, and arginine. In some embodiments, G¹ is a radical of a folate analog of any one of formulas V-X as described herein. In some embodiments, G¹ is a radical of an antifolate of any one of formulas in Table 4 as described herein.

Provided in some embodiments herein are compounds of the formula (I):

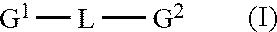

or a pharmaceutically acceptable salt, polymorph, prodrug, solvate or clathrate thereof, wherein:
G¹ is a folate radical of the formula:

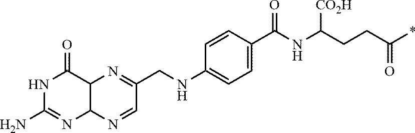

wherein the asterisk denotes the point of attachment of the carbonyl carbon to the linker L; or a pteroyl-amino acid radical;
L is a linker; and
G² is a radical of a corticosteroid.

In some embodiments, G¹ is a radical of a folate analog which is a pyrido[2,3-d]pyrimidine analog ligand, a functional fragment or analog thereof with an affinity (for example, and without limitation, a high specificity) for the folate receptor. For example, such folate analogs may have a relative affinity for binding folate receptor, such as folate receptor beta (FRβ) of about 0.01 or greater as compared to folic acid at a temperature about 20° C./25° C./30° C./physiological temperature.

Specific examples of suitable targeting moieties (or radicals thereof) will now be provided; however, it will be understood that the G¹ targeting moiety (or radical thereof) of the present disclosure may comprise any ligand (or radical thereof) useful to target folate receptors and is not limited to the structures specified herein. The ligand (or radical thereof) may bind to the folate receptor.

As used herein, for example in the following formulae V-XI, a group (such as C(O)Z) that is drawn with a bond extending into a ring but not terminating at a ring atom is understood to be a substituent which substitutes for any hydrogen bonded to a ring atom of that ring. This includes substitution of hydrogens that are explicitly shown or described (e.g., hydrogens as part of X¹-X⁹ groups in formulas V-XI), as well as hydrogens that are not explicitly shown but would be understood to be present.

In some embodiments, G¹ has a structure of formula V or a radical thereof:

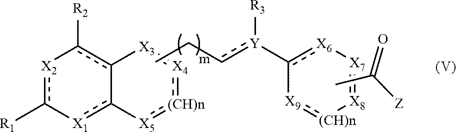

wherein
X₁, X₂, X₃, X₄, X₅, X₆, X₇, X₈, and X₉ are each independently N, NH,
CH, CH₂, O, or S;
Y is C, CH, CH₂, N, NH, O, or S;
Z is glutamic acid or valine;
R₁ and R₂ are each independently NH₂, OH, SH, CH₃, or H;
R₃ is H or an alkyl;
m and n are each independently 0, 1, or between 0 and 1; and

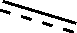

is representative of either a single or double bond C-C.

In some embodiments, formula V has a structure of VI or a radical thereof:

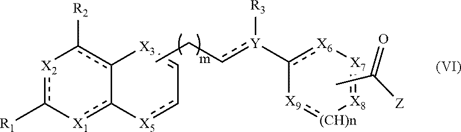

wherein
X₁, X₂, X₃, X₅, X₆, X₇, X₈, and X₉ are each independently N, NH, CH,
CH₂, O, or S;
Y is C, CH, CH₂, N, NH, O, or S;
Z is glutamic acid or valine;
R₁ and R₂ are each independently NH₂, OH, SH, CH₃, or H;
R₃ is H or an alkyl;
m and n are each independently 0, 1, or between 0 and 1; and

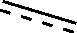

is representative of either a single or double bond C-C.

In some embodiments, formula V has a structure of formula VII or a radical thereof:

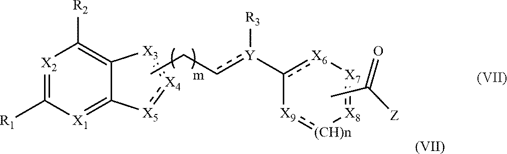

wherein
X₁, X₂, X₃, X₄, X₅, X₆, X₇, X₈, and X₉ are each independently N, NH,
CH, CH₂, O, or S;
Y is C, CH, CH₂, N, NH, O, or S;
Z is glutamic acid or valine;
R₁ and R₂ are each independently NH₂, OH, SH, CH₃, or H;
R₃ is H or an alkyl;
m and n are each independently 0, 1, or between 0 and 1; and

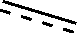

is representative of either a single or double bond C-C.

In some embodiments, formula V has the structure of formula VIII or a radical thereof:

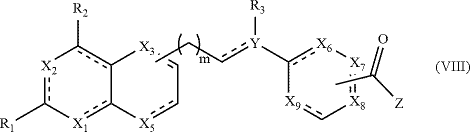

wherein
X₁, X₂, X₃, X₅, X₆, X₇, X₈, and X₉ are each independently N, NH, CH,
CH₂, O, or S;
Y is C, CH, CH₂, N, NH, O, or S;
Z is glutamic acid or valine;
R₁ and R₂ are each independently NH₂, OH, SH, CH₃, or H;
R₃ is H or an alkyl;
m is 0, 1, or between 0 and 1; and

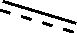 [SMILES: Nc1nc(O)c2nc(CNc3ccc(C(=O)N[C@@H](CCC(=O)O)C(=O)O)cc3)cnc2n1]

is representative of either a single or double bond C-C.

In some embodiments, formula V has the structure of formula IX or ra radical thereof:

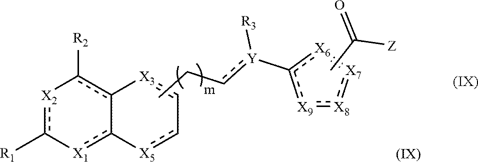

wherein
X₁, X₂, X₃, X₅, X₆, X₇, X₈, and X₉ are each independently N, NH, CH,
CH₂, O, or S;
Y is C, CH, CH₂, N, NH, O, or S;
Z is glutamic acid or valine;
R₁ and _{R2} are each independently NH₂, OH, SH, CH₃, or H;
R₃ is H or an alkyl;
m is 0, 1, or between 0 and 1; and

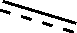 [SMILES: Nc1nc(=O)c2c([nH]1)NCC(CNc1ccc(C(=O)N[C@@H](CCC(=O)O)C(=O)O)cc1)N2C=O]

is representative of either a single or double bond C-C.

In some embodiments, formula V has the structure of formula X or a radical thereof:

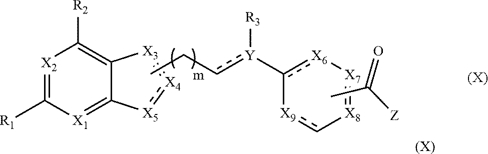

wherein
X₁, X₂, X₃, X₄, X₅, X₆, X₇, X₈, and X₉ are each independently N, NH,
CH, CH₂, O, or S;
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
m is 0, 1, or between 0 and 1; and

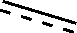 [SMILES: C#CCC(Cc1cnc2nc(N)nc(N)c2n1)c1ccc(C(=O)N[C@@H](CCC(=O)O)C(=O)O)cc1]

is representative of either a single or double bond C-C.

In some embodiments, formula V has the structure of formula XI or a radical thereof:

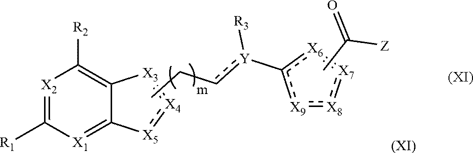

wherein
X₁, X₂, X₃, X₄, X₅, X₆, X₇, X₈, and X₉ are each independently N, NH,
CH, CH₂, O, or S;
Y is C, CH, CH₂, N, NH, O, or S; Z is glutamic acid, valine, or a substrate;
R₁ and R₂ are each independently NH₂, OH, SH, CH₃, or H;
R₃ is hours or an alkyl;
m is 0, 1, or between 0 and 1; and

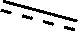 [SMILES: C#CCN(Cc1ccc2nc(N)[nH]c(=O)c2c1)c1ccc(C(=O)N[C@@H](CCC(=O)O)C(=O)O)cc1]

is representative of either a single or double bond C-C.

The chemical structures and spectroscopic data of some additional embodiments of a G¹ moiety (e.g., or radicals thereof) of the present disclosure are provided in the following Table 1, Table 2, Table 3, and Table 4.

Table 1 provides non-limiting examples of additional embodiments of formula VIII.

TABLE 1

Formula VIII

| | |
|---|---|
| Ligand | Structure |
| a | 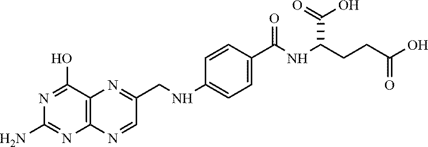 |
| b | 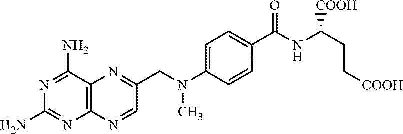 |
| c | 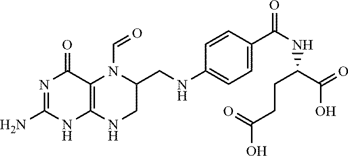 |
| d | 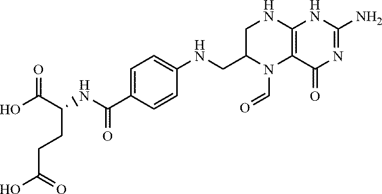 |
| e | 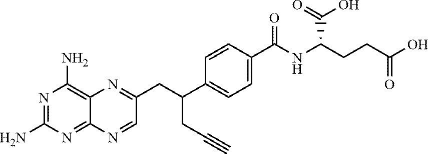 |
| f | 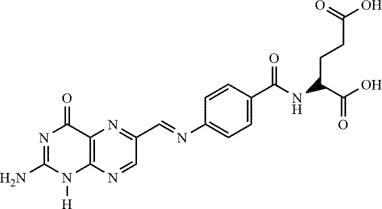 |
| g | 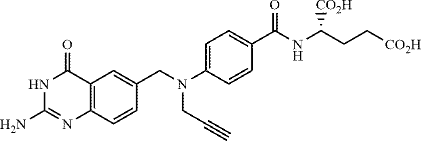 |
| h | 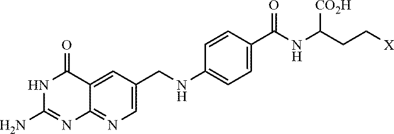 wherein X is PO(OH)₂, CH₂NH₂, or SO₂OH |
| i | 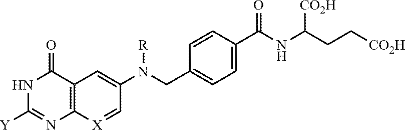 wherein X is N or CH; Y is NH₂, H, or CH₃ and R is H, CH₃, or CHO |
| j | 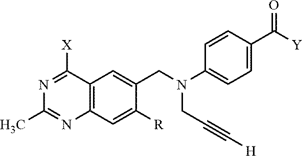 [SMILES: Nc1nc2[nH]cc(CCc3ccc(C(=O)NC(C(=O)O)C(=O)O)cc3)c2c(=O)[nH]1] |
| | wherein X is OH or OCH₃; R is H or CH₃; and Y is glutamic acid or valine. |

Table 2 provides non-limiting examples of additional embodiments of formula IX.

TABLE 2

Formula IX

| | |
|---|---|
| Ligand | Structure |
| aa | 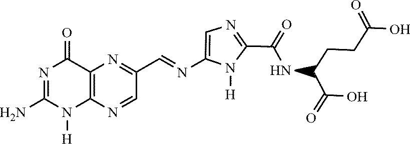 |
| bb | 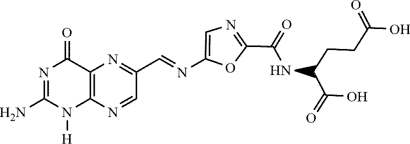 |
| cc | 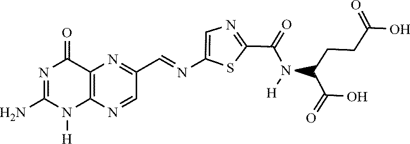 |
| dd | 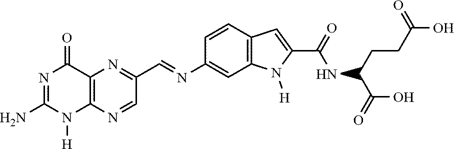 |
| ee | 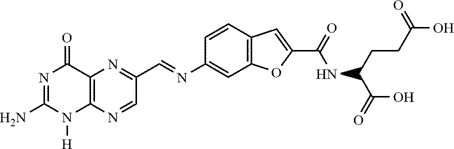 |
| ff | 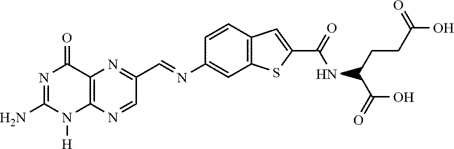 |

Table 3 provides non-limiting examples of additional embodiments of formula X.

TABLE 3

Formula X

| | |
|---|---|
| Ligand | Structure |
| aaa | 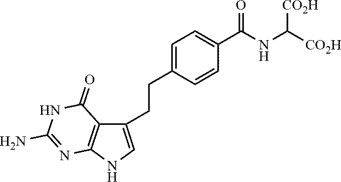 |
| bbb | 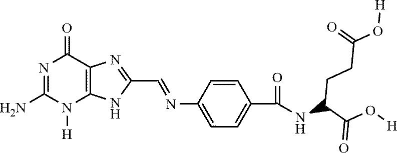 |
| ccc | 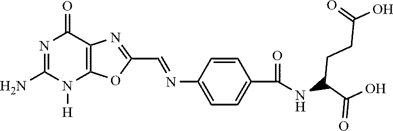 |
| ddd | 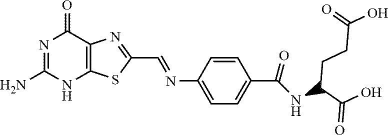 |

In some embodiments, instead of a folate, the G¹ moiety (e.g., a radical thereof) is a nonclassical antifolate such as, for example, pyrido[2,3-d]pyrimidine or similar analogs (or radicals thereof) having the formulas (e.g., radicals of the formulas) set forth in Table 4 below (or an analog or functional fragment thereof). Note that in Table 4, a substituent of 2′,3′-(C₄H₄) indicates an aryl ring fused at the 2′ and 3′ positions of the substituted aryl ring to form a bicyclic aryl system.

TABLE 4

Nonclassical antifolate analogs

| Ligand | Formula |
|---|---|
| aaaa | 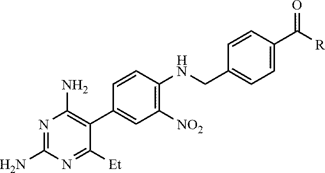 |
| | wherein R is NH₂, NHMe, NHCH(CO₂Et)(CH₂)₂CO₂Et, NHCH(CO₂Et)(CH₂)₂CO₂H, or |
| | 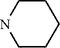 |
| bbbb | 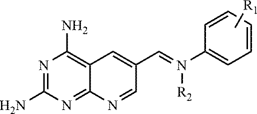 |
| | wherein R₁ is 3,4,5-(OCH₃)₃, 3,4-(OCH₃)₂, or 4-OCH₃; R₂ is H, alkyl chain, or CHO; and is representative of either a single or double bond CC. |
| cccc | 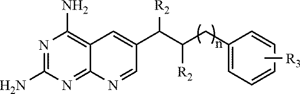 |
| | wherein n is 0 or 1; R₁ and R₂ are each independently an hours or an alkyl; and R₃ is H, 3′,4′,5′-OMe, 2′,3′,4′-OMe, 2′,4′,5′-OMe, 2′,4′,6′-OMe, 3′,4′-OMe, 3′,5′-OMe, 2′,5′-OMe, 2′,3′-C₄H₄, 4′-OMe,2′,3′-C₄H₄, 6′-OMe,2′,3′-C₄H₄, 4′-O-C₆H₅, or 4′-CONH-L-glutamic acid. |
| dddd | 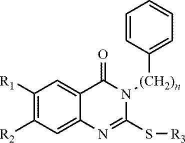 |
| | wherein n is 0 or 1; R₁ is CH₃, Cl or OCH₃; R₂ is H or OCH₃; and R₃ is |
| | 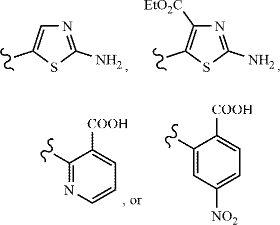 |
| eeee | 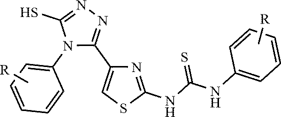 |
| | wherein R is H, 4-Cl, 2-CH₃O, 4-CH₃O, 2,4-(CH₃O)₂, 4-CH₃, or 4-C₆H₅O. |
| ffff | 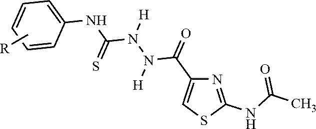 |
| | wherein R is H, 4-Cl, 2-CH₃O, 4-CH₃O, 2,4-(CH₃O)₂, 4-CH₃, or 4-C₆H₅O. |
| gggg | 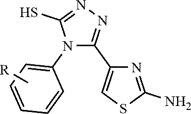 |
| | wherein R is H, 4-Cl, 2-CH₃O, 4-CH₃O, 2,4-(CH₃O)₂, 4-CH₃, or 4-C₆H₅O. |
| hhhh | 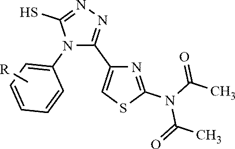 |
| | wherein R is H, 4-Cl, 2-CH₃O, 4-CH₃O, 2,4-(CH₃O)₂, 4-CH₃, or 4-C₆H₅O. |
| iiii | 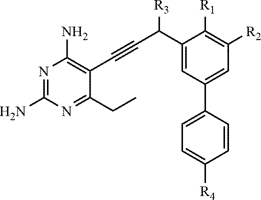 |
| | wherein R₁ and R₂ are each independently H or OMe; R₃ is H or an alkyl; and R₄ is o-COOH or m-COOH or p-COOH. |
| jjjj | 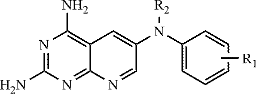 |
| | wherein R₁ is H, 2′-OMe, 4′-OMe,2′,5′-diOMe, 3′,4′,5′-triOMe, 4′-Me, 4′-i-Pr, 3′,4′-(C₄H₄), 2′,3′-(C₄H₄), 4′-NO₂, 2′,5′-diF or 3′,4′,5′-triF; and R₂ is H or an alkyl. |

The steroid present in the compounds described herein can be any suitable steroid that polarizes macrophages from pro-inflammatory (M1) to anti-inflammatory (M2). In some embodiments, G² is a radical of a corticosteroid. Examples of such corticosteroids include betamethasone, cortisone, cortivazol, difluprednate, hydrocortisone, prednisolone, methylprednisolone, prednisone, dexamethasone, hydrocortisone-17-valerate, fluorocortisone, fludrocortisone, paramethasone, eplerenone, amcinonide, alclometasone, beclomethasone, ciclesonide, clobetasol, clocortolone, desonide, deflazacort, desoximetasone, diflorasone, fluocinolone, fluocinonide, fluprednidene, flunisolide, fluticasone, fluclorolone, fludroxycortide, fluorometholone, flucorolone, halcinonide, halometasone, 21-hydroxypregnenolone, halobetacol, loteprednol, meprednisone, mometasone, prednicarbate, prebediolone, rimexolone, tixocortol, triamcinolone, or an ester of any of the foregoing.

In some embodiments, G² is a radical of a steroid selected from the group consisting of betamethasone, cortisone, cortivazol, difluprednate, hydrocortisone, prednisolone, methylprednisolone, prednisone, dexamethasone, hydrocortisone-17-valerate, budesonide, flumethazone, fluticasone proprionate, fluorocortisone, fludrocortisone, paramethasone, eplerenone, and an ester of any of the foregoing. In some embodiments, the steroid is betamethasone. In some embodiments, the steroid is cortisone. In some embodiments, the steroid is cortivazol. In some embodiments, the steroid is difluprednate. In some embodiments, the steroid is hydrocortisone. In some embodiments, the steroid is prednisolone. In some embodiments, the steroid is methylprednisolone. In some embodiments, the steroid is prednisone. In some embodiments, the steroid is dexamethasone. In some embodiments, the steroid is hydrocortisone-17-valerate. In some embodiments, the steroid is budesonide. In some embodiments, the steroid is flumethazone. In some embodiments, the steroid is fluticasone propionate. In some embodiments, the steroid is fluorocortisone. In some embodiments, the steroid is fludrocortisone. In some embodiments, the steroid is paramethasone. In some embodiments, the steroid is eplerenone. In some embodiments, the steroid is amcinonide. In some embodiments, the steroid is alclometasone. In some embodiments, the steroid is beclomethasone. In some embodiments, the steroid is ciclesonide. In some embodiments, the steroid is clobetasol. In some embodiments, the steroid is clocortolone. In some embodiments, the steroid is desonide. In some embodiments, the steroid is deflazacort. In some embodiments, the steroid is desoximetasone. In some embodiments, the steroid is diflorasone. In some embodiments, the steroid is fluocinolone. In some embodiments, the steroid is fluocinonide. In some embodiments, the steroid is fluprednidene. In some embodiments, the steroid is flunisolide. In some embodiments, the steroid is fluticasone. In some embodiments, the steroid is fluclorolone. In some embodiments, the steroid is fludroxycortide. In some embodiments, the steroid is fluorometholone. In some embodiments, the steroid is flucorolone. In some embodiments, the steroid is halcinonide. In some embodiments, the steroid is halometasone. In some embodiments, the steroid is 21-hydroxypregnenolone. In some embodiments, the steroid is halobetacol. In some embodiments, the steroid is loteprednol. In some embodiments, the steroid is meprednisone. In some embodiments, the steroid is mometasone. In some embodiments, the steroid is prednicarbate. In some embodiments, the steroid is prebediolone. In some embodiments, the steroid is rimexolone. In some embodiments, the steroid is tixocortol. In some embodiments, the steroid is triamcinolone.

The linker present in the compounds described herein can be any suitable linker. For example, the linker can be a hydrophilic linker, such as a linker that comprises one or more of an amino acid (which can be the same or different), an alkyl chain, a polyethylene glycol (PEG) monomer, a PEG oligomer, a PEG polymer, or a combination of an any of the foregoing. The linker can comprise an oligomer of peptidoglycans, glycans, or anions. In some embodiments, when the linker comprises a chemical group, that group includes one or more of its atoms in the backbone of the linker. In some embodiments said chemical group is not be required to include atoms in the backbone of L when the group is for binding purposes (such as an albumin binding group), is a glucuronide, or is a “W” group as described herein. For a linker that comprises one or more PEG units, all carbon and oxygen atoms of the PEG units are part of the backbone unless otherwise specified. A cleavable bond for a releasable linker is part of the backbone. The “backbone” of the linker L is the shortest chain of contiguous atoms forming a covalently bonded connection between G¹ and G². In some embodiments, a polyvalent linker has a branched backbone, with each branch serving as a section of backbone linker until reaching a terminus.

The L groups described herein can have any suitable length and chemical composition. For example, L can have a chain length of at least about 7 atoms in length. In some embodiments, L is at least about 10 atoms in length. In some embodiments, L is at least about 14 atoms in length. In some embodiments, L is between about 7 and about 31 (such as, about 7 to 31, 7 to about 31 or 7 to 31) between about 7 and about 24 (such as, about 7 to 24, 7 to about 24 or 7 to 24), or between about 7 and about 20 (such as, about 7 to 20, 7 to about 20 or 7 to 20) atoms in length. In some embodiments, L is between about 14 and about 31 (such as, about 14 to 31, 14 to about 31 or 14 to 31), between about 14 and about 24 (such as, about 14 to 24, 14 to about 24 or 14 to 24), or between about 14 and about 20 (such as, about 14 to 20, 14 to about 20 or 14 to 20) atoms in length. In some embodiments, L has a chain length of at least 7 atoms, at least 14 atoms, at least 20 atoms, at least 25 atoms, at least 30 atoms, at least 40 atoms; or from 1 to 15 atoms, 1 to 5 atoms, 5 to 10 atoms, 5 to 20 atoms, 10 to 40 atoms or 25 to 100 atoms. An example of an L linker group having a chain length of 1 to 5 atoms is a group of the formula:

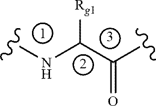

wherein R_{z1} is H, alkyl, arylalkyl, or -alkyl-S-alkyl or the side-chain of any naturally- or non-naturally occurring amino acid, and the like; and the numbers represent the atoms that are counted as being part of the chain, which in this case is 3 atoms. Examples of R_{z1} include H (i.e., side chain of glycine), alkyl (e.g., side chain of alanine, valine, isoleucine, and leucine), -alkyl-S-alkyl (e.g., side chain of methionine), arylalkyl (e.g., side chain of phenylalanine, tyrosine, and tryptophan), and the like. In some embodiments, the atom to which R_{z1} is attached is chiral and can have any suitable relative configuration, such as a D-or L- configuration.

The atoms used in forming L can be combined in all chemically relevant ways, such as chains of carbon atoms forming alkylene groups, chains of carbon and oxygen atoms forming polyoxyalkylene groups, chains of carbon and nitrogen atoms forming polyamines, and others. In addition, it is to be understood that the bonds connecting atoms in the chain may be either saturated or unsaturated, such that for example, alkanes, alkenes, alkynes, cycloalkanes, arylenes, imides, and the like may be divalent radicals that are included in L. In addition, it is to be understood that the atoms forming the linker may also be cyclized upon each other to form saturated or unsaturated divalent cyclic radicals in the linker, such as radicals of the formulae:

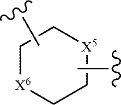

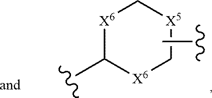

wherein each X⁵ is independently CH₂, N (when there is a bond attached to X⁵), NH or O and each X⁶ is independently N, C (when there is a bond attached to X⁶) or CH. Thus, for example, the foregoing groups can be of the formulae:

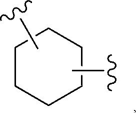

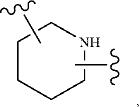

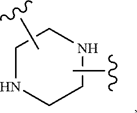

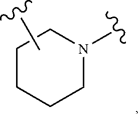

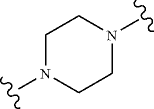

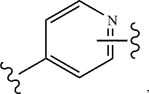

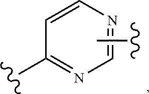

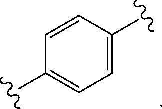

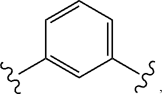

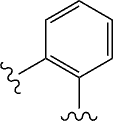

and the like. In each of the foregoing and other L groups described herein, the chain forming the linker may be substituted or unsubstituted.

Alternatively, or in addition to chain length, in some embodiments L has suitable substituents that can affect the hydrophobicity or hydrophilicity of L. Thus, for example, L can have a hydrophobic side chain group, such as an alkyl, cycloalkyl, aryl, arylalkyl, or like group, each of which is optionally substituted. If L were to include one or more amino acids, L can contain hydrophobic amino acid side chains, such as one or more amino acid side chains from phenylalanine (Phe) and tyrosine (Tyr), including substituted variants thereof, and analogs and derivatives of such side chains. Variants, analogs, and derivatives of these side chains include, for example, groups such as:

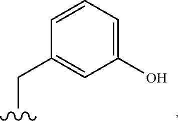

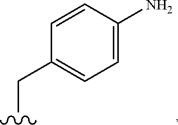

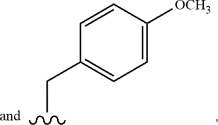

which are respectively a variant of tyrosine, an amine analog of tyrosine, and a methoxy derivative of tyrosine. Other variants, analogs, and derivatives are contemplated.

In some embodiments, L comprises portions that are neutral under physiological conditions. In some embodiments, L comprises portions that can be protonated or deprotonated to carry one or more positive or one or more negative charges, respectively. In some embodiments, L comprises neutral portions and portions that may be protonated to carry one or more positive charges. Examples of neutral portions include poly hydroxyl groups, such as sugars, carbohydrates, saccharides, inositols, and the like, and/or polyether groups, such as polyoxyalkylene groups including polyoxyethylene, polyoxypropylene, and the like. Examples of portions that can be protonated to carry one or more positive charges include amino groups, such as polyaminoalkylenes including ethylene diamines, propylene diamines, butylene diamines and the like, and/or heterocycles including pyrrolidines, piperidines, piperazines, and other amino groups, each of which can be optionally substituted. Examples of portions that can be deprotonated to carry one or more negative charges include carboxylic acids, such as aspartic acid, glutamic acid, and longer chain carboxylic acid groups, and sulfuric acid esters, such as alkyl esters of sulfuric acid.

Illustrative polyoxyalkylene groups include those of a specific length range from about 4 to about 20 polyoxyalkylene (e.g., polyethylene glycol) groups, such as about 4 to 20, 4 to about 20 or 4 to 20 polyoxyalkylene groups. Illustrative alkyl sulfuric acid esters may also be introduced with click chemistry directly into the backbone. Illustrative L groups comprising polyamines include L groups comprising EDTA and DTPA radicals:

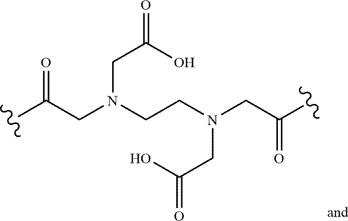

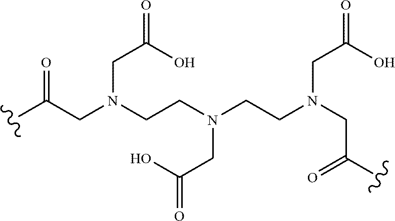

(poly)peptides:

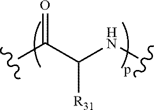

β-amino acids, and the like:

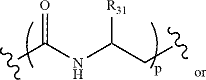

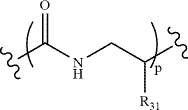

and combinations thereof, wherein each R₃₁ is independently H, alkyl, arylalkyl, heterocyclylalkyl, ureido, aminoalkyl, alkylthio or amidoalkyl, such as in the side chains of naturally-occurring amino acids like alanine, valine, leucine, isoleucine, phenylalanine, tyrosine, tryptophan, serine, threonine, asparagine, methionine, lysine, arginine, and histidine. Non-naturally occurring amino acids are also contemplated herein.

As discussed herein, in some embodiments L includes at least one releasable portion. In some embodiments, L includes at least two releasable linkers (e.g., cleavable linkers). The choice of a releasable linker or a non-releasable linker can be made independently for each application or configuration of the compounds described herein. The releasable linkers described herein comprise various atoms, chains of atoms, functional groups, and combinations of functional groups. For example, in some embodiments the releasable linker comprises about 1 to about 30 atoms (e.g., about 1 to 30, 1 to about 30, and 1 to 30 atoms), or about 2 to about 20 atoms (e.g., about 2 to 20, 2 to about 20, and 2 to 20 atoms). Lower molecular weight linkers (e.g., those having an approximate molecular weight of about 30 g/mol to about 1,000 g/mol, such as from about 30 g/mol to about 300 g/mol, about 100 g/mol to about 500 g/mol or about 150 g/mol to about 600 g/mol) are also described. Precursors to such linkers may be selected to have either nucleophilic or electrophilic functional groups, or both, optionally in a protected form with a readily cleavable protecting group to facilitate their use in synthesis of the intermediate species.

In some embodiments, the linker is a bivalent linker (e.g., connecting a single G¹ to a single G²). In some embodiments, the linker is a multivalent linker (e.g., connecting two or more G¹ to a single G²). In some embodiments, the linker is polyvalent and has multiple attachment points for one or more additional chemical groups (e.g., the additional chemical groups comprise one or more additional G¹ groups; or the additional chemical groups comprise one or more binding ligands that are not G¹ groups). In some embodiments, the linker is a releasable linker. In some embodiments, the linker is a non-releasable linker.

In some embodiments, L is (L¹)ₒ-Y-(L²)ₚ, wherein:
each L¹ is a first linker;
each L² is a second linker;
Y is a template that connects multiple arms of the compound;
o is an integer from 1-5; and
p is an integer from 1-5.

In some embodiments, L¹ and L² are the same. In some embodiments, L¹ and L² are different. In some embodiments, each L¹ is connected to an G¹ group (and the Y group). In certain embodiments, each L² is connected to a G² group (and the Y group). In certain embodiments, o and m are the same, such as 1-6, 1-3, or 1. In some embodiments, p is 1. In some embodiments, o is 1. In some embodiments, p and o are each 1.

In some embodiments, each L¹ and L² independently comprise a oligoethylene glycol (chain), a polyethylene glycol (chain), an alkyl (chain), an oligopeptide (chain), or a polypeptide (chain). In some embodiments, each L¹ and L² independently comprise an oligoethylene glycol (chain) or a polyethylene glycol (chain).

In some embodiments, each L¹ and L² independently comprise a triazole or an amide.

In some embodiments, each L¹ and L² independently comprise an oligopeptide (chain) or a polypeptide (chain). In some embodiments, each L¹ and L² independently comprise a peptidoglycan (chain).

In some embodiments, each L¹ and L² independently comprise an oligoproline or an oligopiperidine.

In some embodiments, each L¹ and L² are independently a length from 15-200 angstroms (Å).

In some embodiments, o is an integer from 1-5. In some embodiments, o is an integer from 1-3. In some embodiments, o is 1. In some embodiments, o is m.

In some embodiments, p is an inter from 1-5. In some embodiments, p is an integer from 1-3. In some embodiments, p is 1.

Provided in some embodiments herein is a multivalent compound having the following formula:

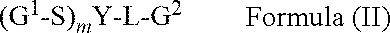

wherein:
G¹ is a folate radical, an antifolate radical, or a folate analog radical;
S is a spacer (e.g., having a length for the arms of the multivalent targeting ligand (e.g., drug) to reach multiple adjacent folate receptors on a target cell);
Y is a template that connects multiple arms of the compound;
L is a (e.g., bi-functionalized) linker connecting G¹ to G² (e.g., through a first covalent bond connecting L to G¹ and a second covalent bond linking L to G²); and
G² is a radical of a steroid; and
m is 2-6.

In some embodiments, the spacer is the optimal length for the arms of the multivalent drug to reach to multiple adjacent folate receptors on a target (e.g., macrophage) cell.

In some embodiments, S comprises an oligoethylene, a polyethyleneglycol, an alkyl chain, an oligopeptide or a polypeptide. In some embodiments, S is an oligoethylene glycol or a polyethylene glycol

In some embodiments, S is an oligopeptide or polypeptide.

In embodiments, S is a peptidoglycan.

In some embodiments, the spacer is a rigid linker. In some embodiments, S is a rigid linker, such as, for example, an oligoproline or an oligopiperidine

In some embodiments, S is a length of at least 15 angstroms (Å). In some embodiments, S is a length of at most 200 angstroms (Å). In some embodiments, S is a length from 15-200 angstroms (Å).

In some embodiments, Y is a template that connects multiple arms of the compound. In some embodiments, Y has a repeating structure. In some embodiments, Y comprises a releasable bond. In some embodiments, L comprises a disulfide bond. In some embodiments, Y comprises at least one citric acid group (or a radical thereof). In some embodiments, Y comprises one or more triazole. In some embodiments, Y comprises one or more amine. In some embodiments, Y comprises one or more amide. In some embodiments, Y has an aromatic core (e.g., an aryl core or a heteroaryl core). In some embodiments, Y has an alkyl(ene) core. In some embodiments, Y has an amine core. In some embodiments, Y is N(L¹)₃ (e.g., wherein L¹ is described elsewhere herein). In some embodiments, Y is phenyl substituted with three L¹ (e.g., wherein L¹ is described elsewhere herein). In some embodiments, Y is C(L¹)₄ (e.g., wherein L¹ is described elsewhere herein).

In some embodiments, Y is attached to a single L¹. In some embodiments, Y is attached to a single L². In some embodiments, Y is attached to a single L¹ and a single L². In some embodiments, Y is independently connected to each L¹ and L² by an amide bond. In some embodiments, Y is attached to L.

In some embodiments, Y is a template (e.g., a multivalent template) that connects multiple arms of the compound. In some embodiments, Y has a repeating structure. In some embodiments, Y comprises at least one citric acid group (or a radical thereof). In some embodiments, the template has the following structure:

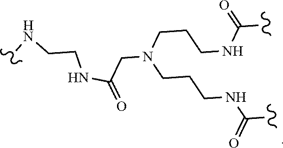

In some embodiments, Y is a template (e.g., a multivalent template) that connects multiple arms of the compound and comprises a template (e.g., a repeating unit) of the following structure:

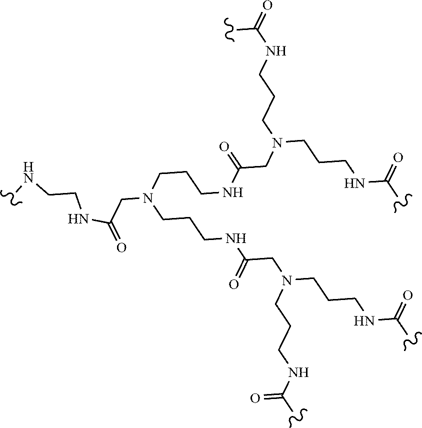

In some embodiments, Y is a template that connects multiple arms of the compound that has a citric acid-based template. In some embodiments, Y is a template (e.g., a multivalent template) that connects multiple arms of the compound and has a (e.g., citric acid-based) template of the following structure:

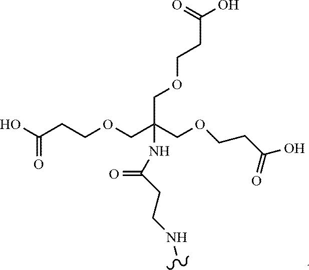

In some embodiments, Y is a template (e.g., a multivalent template) that connects multiple arms of the compound and has a (e.g., citric acid-based) template of the following structure:

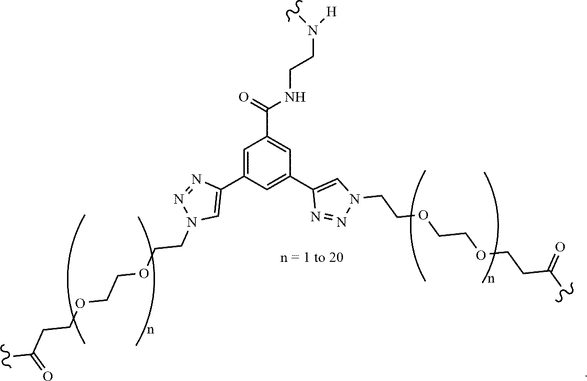

In some embodiments, Y is a template (e.g., a multivalent template) that connects multiple arms of the compound and has a (e.g., citric acid-based) template of the following structure:

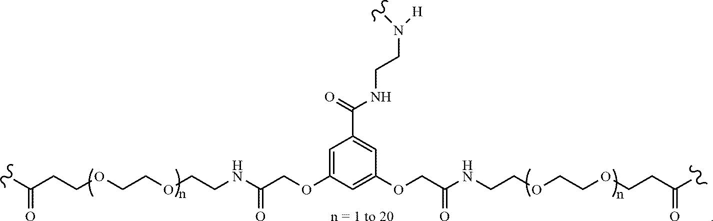

In some embodiments, L comprises at least one linker group, each linker group selected from the group consisting of polyethylene glycol (PEG), alkyl, sugar, and peptide. In some embodiments, the linker is a polyethylene glycol- (PEG-) (e.g., pegylated-), alkyl-, sugar-, and peptide-based dual linker.

In some embodiments, the linker comprises a PEG oligomer with 2-16 PEG units. In some embodiments, the linker comprises a PEG oligomer with 12 PEG units.

In some embodiments, L is a non-releasable linker (e.g., bivalently (e.g., covalently) attached to G² and G¹). In some embodiments, L is a releasable linker (e.g., bivalently (e.g., covalently) attached to G² and G¹).

In one example, L can comprise one or more releasable linkers that cleave under the conditions described herein by a chemical mechanism involving beta elimination. Such releasable linkers include beta-thio, beta-hydroxy, and beta-amino substituted carboxylic acids and derivatives thereof, such as esters, amides, carbonates, carbamates, and ureas. Such linkers also include 2- and 4-thioarylesters, carbamates, and carbonates.

An example of a releasable linker includes a linker of the formula:

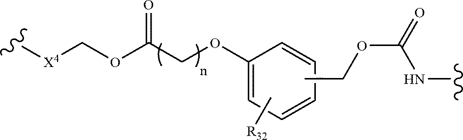

wherein X⁴ is NR, n is an integer selected from 0, 1, 2, and 3, R₃₂ is hydrogen, or a substituent, including a substituent capable of stabilizing a positive charge inductively or by resonance on the aryl ring, such as alkoxy, and the like. The releasable linker can be further substituted.

Assisted cleavage of releasable portions of L can include mechanisms involving benzylium intermediates, benzyne intermediates, lactone cyclization, oxonium intermediates, beta-elimination, and the like. In addition to fragmentation subsequent to cleavage of a releasable portion of L, the initial cleavage of the releasable linker may be facilitated by an anchimerically assisted mechanism. Thus, in the example of a releasable portion of L given above, the hydroxyalkanoic acid, which may cyclize, facilitates cleavage of the methylene bridge, by for example an oxonium ion, and facilitates bond cleavage or subsequent fragmentation after bond cleavage of the releasable linker. Alternatively, acid catalyzed oxonium ion-assisted cleavage of the methylene bridge may begin a cascade of fragmentation of this illustrative bivalent linker, or fragment thereof. Alternatively, acid-catalyzed hydrolysis of the carbamate may facilitate the beta elimination of the hydroxyalkanoic acid, which may cyclize, and facilitate cleavage of methylene bridge, by for example an oxonium ion. It is appreciated that other chemical mechanisms of bond cleavage under the metabolic, physiological, or cellular conditions described herein may initiate such a cascade of fragmentation. It is appreciated that other chemical mechanisms of bond cleavage under the metabolic, physiological, or cellular conditions described herein can initiate such a cascade of fragmentation.

Illustrative mechanisms for cleavage of the bivalent linkers described herein include the following 1,4 and 1,6 fragmentation mechanisms for carbonates and carbamates:

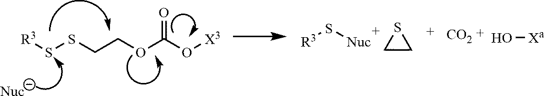

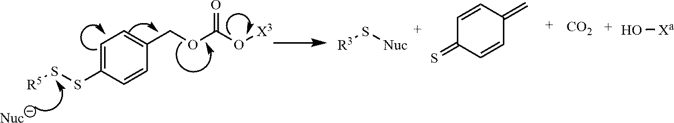

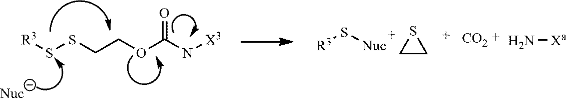

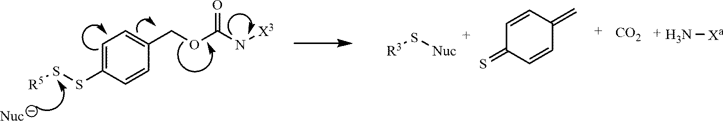

wherein Nuc⁻ is an exogenous or endogenous nucleophile, glutathione, or bioreducing agent, and the like, and R^{a} and X^{a} are connected through other portions of the bivalent linker. The location of R^{a} and X^{a} can be switched such that, e.g., the resulting products are X^{a}-S-Nuc and HO-R^{a} H2N-R^{a}.

Although the above fragmentation mechanisms are depicted as concerted mechanisms, any number of discrete steps can take place to effect the ultimate fragmentation of the bivalent linker to the final products shown. For example, the bond cleavage can also occur by acid catalyzed elimination of the carbamate moiety, which may be anchimerically assisted by the stabilization provided by either the aryl group of the beta sulfur or disulfide illustrated in the above examples. In those variations of this embodiment, the releasable linker is the carbamate moiety. Alternatively, the fragmentation can be initiated by a nucleophilic attack on the disulfide group, causing cleavage to form a thiolate. The thiolate can intermolecularly displace a carbonic acid or carbamic acid moiety and form the corresponding thiacyclopropane. In the case of the benzyl-containing bivalent linkers, following an illustrative cleavage of the disulfide bond, the resulting phenyl thiolate can further fragment to release a carbonic acid or carbamic acid moiety by forming a resonance-stabilized intermediate. In any of these cases, the releasable nature of the illustrative bivalent linkers described herein may be realized by whatever mechanism may be relevant to the chemical, metabolic, physiological, or biological conditions present.

As described above, therefore, releasable linkers can comprise a disulfide group. Further examples of releasable linkers comprised in L include divalent radicals comprising alkyleneaziridin-1-yl, alkylenecarbonylaziridin-1-yl, carbonylalkylaziridin-1-yl, alkylenesulfoxylaziridin-1-yl, sulfoxylalkylaziridin-1-yl, sulfonylalkylaziridin-1-yl, or alkylenesulfonylaziridin-1-yl groups, wherein each of the releasable linkers is optionally substituted. Additional examples of releasable linkers comprised in L include divalent radicals comprising methylene, 1-alkoxyalkylene, 1-alkoxycycloalkylene, 1-alkoxyalkylenecarbonyl, 1-alkoxycycloalkylenecarbonyl, carbonylarylcarbonyl,carbonyl(carboxyaryl) carbonyl, carbonyl(biscarboxyaryl)carbonyl, haloalkylenecarbonyl, alkylene(dialkylsilyl), alkylene(alkylarylsilyl), alkylene(diarylsilyl), (dialkylsilyl)aryl, (alkylarylsilyl)aryl, (diarylsilyl)aryl, oxycarbonyloxy, oxycarbonyloxyalkyl, sulfonyloxy, oxysulfonylalkyl, iminoalkylidenyl, carbonylalkylideniminyl, iminocycloalkylidenyl, carbonylcycloalkylideniminyl, alkylenethio, alkylenearylthio or carbonylalkylthio groups, wherein each of the releasable linkers is optionally substituted.

Additional examples of releasable linkers comprised in L include an oxygen atom and methylene, 1-alkoxyalkylene, 1-alkoxycycloalkylene, 1-alkoxyalkylenecarbonyl or 1-alkoxycycloalkylenecarbonyl groups, wherein each of the releasable linkers is optionally substituted. Alternatively, in some embodiments the releasable linker includes an oxygen atom and a methylene group, wherein the methylene group is substituted with an optionally substituted aryl, and the releasable linker is bonded to the oxygen to form an acetal or ketal. Further, in some embodiments the releasable linker includes an oxygen atom and a sulfonylalkyl group, and the releasable linker is bonded to the oxygen to form an alkylsulfonate.

Additional examples of releasable linkers comprised in L include a nitrogen and iminoalkylidenyl, carbonylalkylideniminyl, iminocycloalkylidenyl, and carbonylcycloalkylideniminyl groups, wherein each of the releasable linkers is optionally substituted and the releasable linker is bonded to the nitrogen to form an hydrazone. In some embodiments, the hydrazone is acylated with a carboxylic acid derivative, an orthoformate derivative, or a carbamoyl derivative to form various acylhydrazone releasable linkers.

Additional examples of releasable linkers comprised in L include an oxygen atom and alkylene(dialkylsilyl), alkylene(alkylarylsilyl), alkylene(diarylsilyl), (dialkylsilyl)aryl, (alkylarylsilyl)aryl or (diarylsilyl)aryl groups, wherein each of the releasable linkers is optionally substituted and the releasable linker is bonded to the oxygen to form a silanol.

Additional examples of releasable linkers comprised in L include two independent nitrogens and carbonylarylcarbonyl, carbonyl(carboxyaryl)carbonyl, or carbonyl(biscarboxyaryl)carbonyl. In some embodiments the releasable linker is bonded to the heteroatom nitrogen to form an amide, and also bonded to X^{a} or R^{a} via an amide bond.

Additional examples of releasable linkers comprised in L include an oxygen atom, a nitrogen, and a carbonylarylcarbonyl, carbonyl(carboxyaryl)carbonyl, or carbonyl(biscarboxyaryl)carbonyl. In some embodiments, the releasable linker forms an amide, and in some embodiments is bonded to X^{a} or R^{a} via an amide bond.

In some embodiments, L comprises an optionally substituted 1-alkylenesuccinimid-3-yl group and a releasable portion comprising methylene, 1-alkoxyalkylene, 1-alkoxycycloalkylene, 1-alkoxyalkylenecarbonyl or 1-alkoxycycloalkylenecarbonyl groups, each of which can be optionally substituted, to form a succinimid-1-ylalkyl acetal or ketal.

In some embodiments, L comprises carbonyl, thionocarbonyl, alkylene, cycloalkylene, alkylenecycloalkyl, alkylenecarbonyl, cycloalkylenecarbonyl, carbonylalkylcarbonyl, 1-alkylenesuccinimid-3-yl, 1-(carbonylalkyl)succinimid-3-yl, alkylenesulfoxyl, sulfonylalkyl, alkylenesulfoxylalkyl, alkylenesulfonylalkyl, carbonyltetrahydro-2H-pyranyl, carbonyltetrahydrofuranyl, 1-(carbonyltetrahydro-2H-pyranyl)succinimid-3-yl or 1-(carbonyltetrahydrofuranyl)succinimid-3-yl, each of which is optionally substituted. In some embodiments, L further comprises an additional nitrogen such that L comprises alkylenecarbonyl, cycloalkylenecarbonyl, carbonylalkylcarbonyl or 1-(carbonylalkyl)succinimid-3-yl groups, each of which is optionally substituted, bonded to the nitrogen to form an amide. In some embodiments, L further comprises a sulfur atom and alkylene or cycloalkylene groups, each of which is optionally substituted with carboxy, and is bonded to the sulfur to form a thiol. In some embodiments, L comprises a sulfur atom and 1-alkylenesuccinimid-3-yl and 1-(carbonylalkyl)succinimid-3-yl groups bonded to the sulfur to form a succinimid-3-ylthiol.

In some embodiments L comprises a nitrogen and a releasable portion comprising alkyleneaziridin-1-yl, carbonylalkylaziridin-1-yl, sulfoxylalkylaziridin-1-yl, or sulfonylalkylaziridin-1-yl, each of which is optionally substituted. In some embodiments, L comprises carbonyl, thionocarbonyl, alkylenecarbonyl, cycloalkylenecarbonyl, carbonylalkylcarbonyl, or 1-(carbonylalkyl)succinimid-3-yl, each of which is optionally substituted, and bonded to the releasable portion to form an aziridine amide.

Examples of L include alkylene-amino-alkylenecarbonyl, alkylene-thio-(carbonylalkylsuccinimid-3-yl), and the like, as further illustrated by the following formulae:

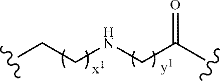

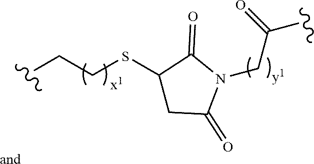

wherein x′ and y′ are each independently 1, 2, 3, 4, or 5.

L can have any suitable assortment of atoms in the chain, including C (e.g., —CH₂—, C(O)), N (e.g., NH, NR^{b}, wherein R^{b} is, e.g., H, alkyl, alkylaryl, and the like), O (e.g., —O—), P (e.g., —O—P(O)(OH)O—), and S (e.g., —S—). For example, the atoms used in forming L may be combined in all chemically relevant ways, such as chains of carbon atoms forming alkyl groups, chains of carbon and oxygen atoms forming polyoxyalkyl groups, chains of carbon and nitrogen atoms forming polyamines, and others, including rings, such as those that form aryl and heterocyclyl groups (e.g., triazoles, oxazoles, and the like). In addition, the bonds connecting atoms in the chain in L may be either saturated or unsaturated, such that for example, alkanes, alkenes, alkynes, cycloalkanes, arylenes, imides, and the like may be divalent radicals that are included in L. Further, the chain-forming L can be substituted or unsubstituted.

Additional examples of L groups include the groups 1-alkylsuccinimid-3-yl, carbonyl, thionocarbonyl, alkyl, cycloalkyl, alkylcycloalkyl, alkylcarbonyl, cycloalkylcarbonyl, carbonylalkylcarbonyl, 1-alkylsuccinimid-3-yl, 1-(carbonylalkyl)succinimid-3-yl, alkylsulfoxyl, sulfonylalkyl, alkylsulfoxylalkyl, alkylsulfonylalkyl, carbonyltetrahydro-2H-pyranyl, carbonyltetrahydrofuranyl, 1-(carbonyltetrahydro-2H-pyranyl)succinimid-3-yl, and 1-(carbonyltetrahydrofuranyl)succinimid-3-yl, wherein each group can be substituted or unsubstituted. Any of the aforementioned groups can be L or can be included as a portion of L. In some instances, any of the aforementioned groups can be used in combination (or more than once) (e.g., -alkyl-C(O)-alkyl) and can further comprise an additional nitrogen (e.g., alkyl-C(O)-NH-, -NH-alkyl- C(O)- or -NH-alkyl-), oxygen (e.g., -alkyl-O-alkyl-) or sulfur (e.g., -alkyl-S-alkyl-). Examples of such L groups are alkylcarbonyl, cycloalkylcarbonyl, carbonylalkylcarbonyl, 1-(carbonylalkyl)succinimid-3-yl, and succinimid-3-ylthiol, wherein each group can be substituted or unsubstituted.

In some embodiments, L is formed via click chemistry/click chemistry-derived. Those of skill in the art understand that the terms “click chemistry” and “click chemistry-derived” generally refer to a class of small molecule reactions commonly used in conjugation, allowing the joining of substrates of choice with specific molecules. Click chemistry is not a single specific reaction, but describes a way of generating products that follow examples in nature, which also generates substances by joining small modular units. In many applications, click reactions join a biomolecule and a reporter molecule. Click chemistry is not limited to biological conditions: the concept of a “click” reaction has been used in pharmacological and various biomimetic applications. However, they have been made notably useful in the detection, localization and qualification of biomolecules.

Click reactions can occur in one pot, typically are not disturbed by water, can generate minimal byproducts, and are “spring-loaded″-characterized by a high thermodynamic driving force that drives it quickly and irreversibly to high yield of a single reaction product, with high reaction specificity (in some cases, with both regio- and stereo-specificity). These qualities make click reactions suitable to the problem of isolating and targeting molecules in complex biological environments. In such environments, products accordingly need to be physiologically stable and any byproducts need to be non-toxic (for in vivo systems).

Click chemistry examples include examples where L can be derived from copper-catalyzed azide-alkyne cycloaddition (CuAAC), strain-promoted azide-alkyne cycloaddition (SPAAC), inverse electron demand Diels- Alder reaction (IEDDA), and Staudinger ligation (SL). For example, X^{a} and R^{a} can be linked to each other as shown in Schemes 1-5:

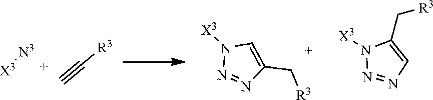

Scheme 1

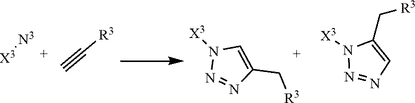

Scheme 2

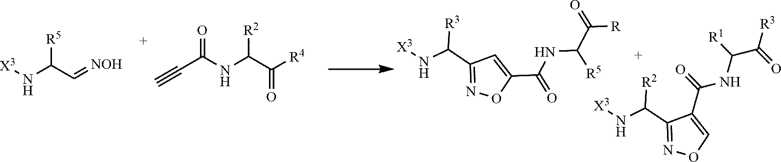

Scheme 3

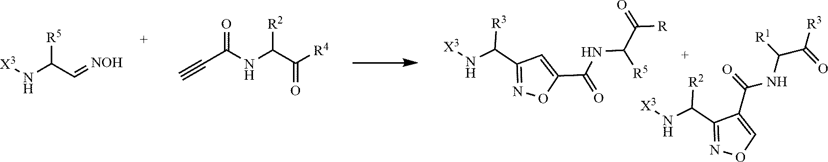

Scheme 4

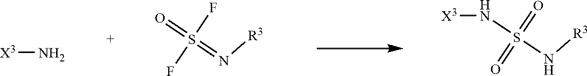

Scheme 5

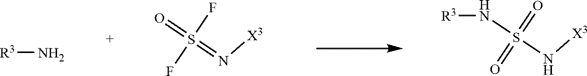

wherein each R^{b} is independently H, alkyl, arylalkyl, -alkyl-S-alkyl or arylalkyl or the side-chain of any naturally- or non-naturally occurring amino acid and the like. In Schemes 1-5, the wavy line connected to X^{a} and R^{a} represents a linkage between X^{a} and R^{a} and the groups to which they are attached. It should be appreciated that in Schemes 1-5, the triazole, oxazole, and the —NH—SO2—NH— group would be considered to be part of L.

In some embodiments, L is a linker selected from the group consisting of pegylated-, alkyl-, sugar-, and peptide- based dual linker; L is either a non-releasable linker or a releasable linker bivalently covalently attached to the folate ligand (or, in other embodiments, folate analogue or antifolate) and the steroid.

In some embodiments, L is:

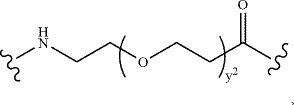

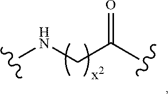

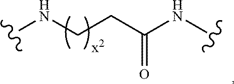

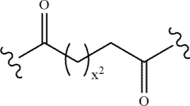

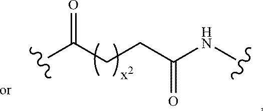

wherein x″ is an integer from 0 to 10, and y″ is an integer from 3 to 100.

In some aspects, x″ is an integer from 3 to 10.

In some embodiments, L is:

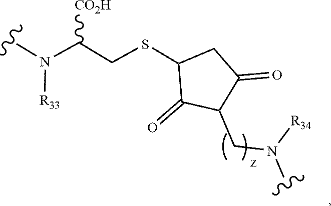

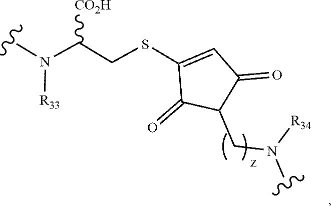

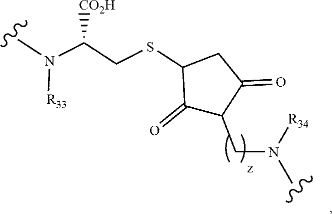

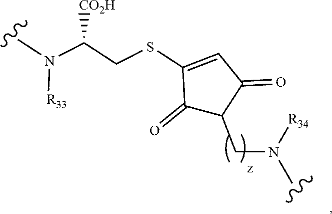

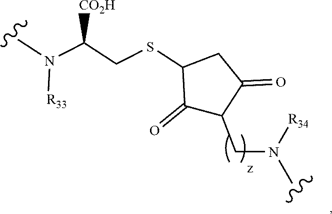

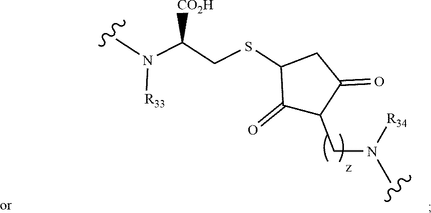

wherein each of R₃₃ and R₃₄ is independently H or C₁-C₆ alkyl; and z is an integer from 1 to 8.

In some embodiments, L is:

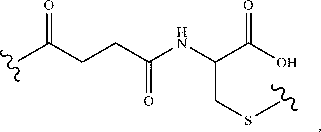

In some embodiments, L is:

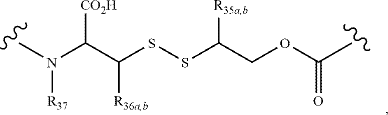

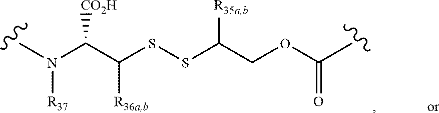

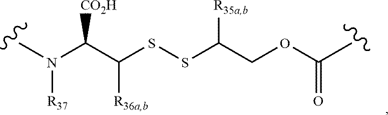

wherein R₃₇ is H or C₁-C₆ alkyl; R₃₅ₐ, R_{35b}, R₃₆ₐ, and R_{36b} each is independently H or C₁-C₆ alkyl.

In some embodiments, L comprises an amino acid. In some embodiments, L comprises an amino acid selected from the group consisting of Lys, Asn, Thr, Ser, Ile, Met, Pro, His, Gln, Arg, Gly, Asp, Glu, Ala, Val, Phe, Leu, Tyr, Cys, and Trp. In some embodiments, L comprises at least two amino acids independently selected from the group consisting of Glu and Cys. In some embodiments, L comprises Glu-Glu, wherein the glutamic acids are covalently bonded to each other through the carboxylic acid side chains.

In some embodiments, L comprises one or more hydrophilic spacer linkers comprising a plurality of hydroxyl functional groups.

In some embodiments, L comprises at least one 2,3-diaminopropionic acid group, at least one glutamic acid group (e.g., unnatural amino acid D-Glutamic acid), and at least one cysteine group. One example of such a linker is one having the non-natural amino acid, such as a linker having the repeating unit of the formula:

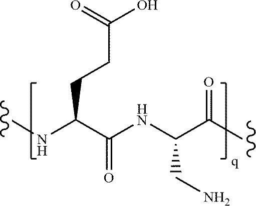

wherein q is an integer from 1 to 10 (e.g., 1 to 3 and 2 to 5). In some embodiments, L comprises the general formula:

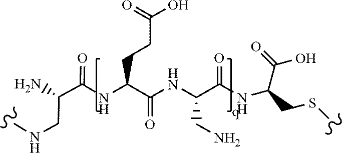

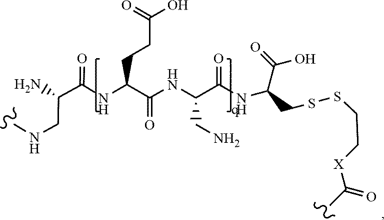

wherein X can be O, NH, NR, or S, and q is an integer from 1 to 10. In some embodiments, L comprises the formula:

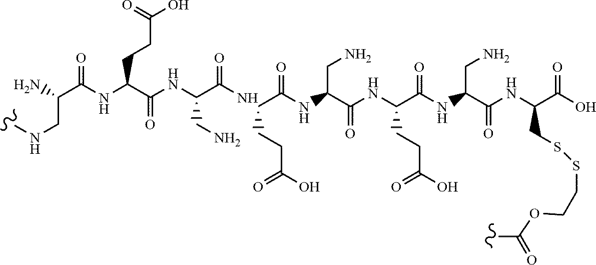

wherein the disulfide group is a part of a self-immolative group that can be generically described as a group of the formula -CH₂-S-S-CH₂-.

In some embodiments, the compounds described herein include linkages that cause the steroids described herein to be released by any suitable mechanism, including a release mechanism involving reduction, oxidation, or hydrolysis. An example of a reduction mechanism includes reduction of a disulfide group into two separate sulfyhydryl groups. Thus, for example, a group of the formula -CH₂-S-S-CH₂- would be reduced to two separate groups of the formula -CH₂-SH, such that if the linker were of the formula:

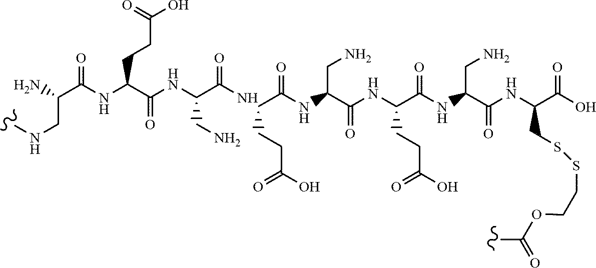

the reduction product would be of the formula:

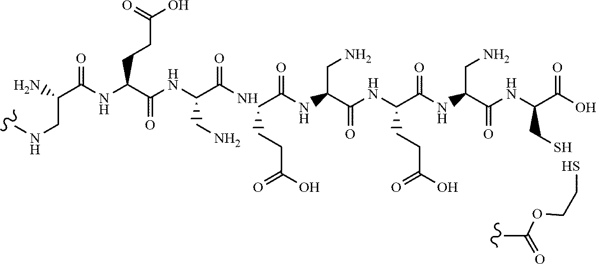

In this example, the steroid is attached to the linker via a self-immolative moiety (e.g., a disulfide group).

An example of a self-immolative disulfide also includes a sterically protected disulfide bond. The steroid can be attached to the linker via any other suitable self-immolative bond, including via a self-immolative cathepsin cleavable amino acid sequence; via a self-immolative furin cleavable amino acid sequence; via a self-immolative β-glucuronidase cleavable moiety; via a self-immolative phosphatase cleavable moiety; or via a self-immolative sulfatase cleavable moiety. Multiple self-immolative linkages are also contemplated herein.

In some embodiments, the linker comprises a self-immolative moiety. In some embodiments, the linker comprises a self-immolative disulfide and or sterically protected disulfide bond. In some embodiments, the linker comprises a self-immolative cathepsin-cleavable amino acid sequence. In some embodiments, the linker comprises a self-immolative furin-cleavable amino acid sequence. In some embodiments, the linker comprises a self-immolative β-glucuronidase-cleavable moiety. In some embodiments, the linker comprises a self-immolative phosphatase-cleavable moiety. In some embodiments, the linker comprises a self-immolative sulfatase-cleavable moiety.

In some embodiments, the linker comprises a phosphate or pyrophosphate group. In some embodiments, the linker comprises a cathepsin B cleavable group. In some embodiments, the cathepsin B cleavable group is Valine-Citrulline. In some embodiments, the linker comprises a carbamate moiety. In some embodiments, the linker comprises a β-glucuronide.

In some embodiments, the compounds described herein include linkages where the steroid is attached to the linker via an ester, phosphate, oxime, acetal, pyrophosphate, polyphosphate, disulfide, sulfate, hydrazide, imine, carbonate, carbamate or enzyme-cleavable amino acid sequence.

In some embodiments, the linker comprises an ester, phosphate, oxime, acetal, pyrophosphate, polyphosphate, disulfide, sulfate, hydrazide, imine, carbonate, carbamate or enzymelcleavable amino acid sequence

In some embodiments, L comprises one or more spacer linkers. Spacer linkers can be hydrophilic spacer linkers comprising a plurality of hydroxyl functional groups. A spacer “L” can comprise any stable arrangement of atoms. A spacer comprises one or more L′. Each L′ is independently selected from the group consisting an amide, ester, urea, carbonate, carbamate, disulfide, amino acid, amine, ether, alkyl, alkene, alkyne, heteroalkyl (e.g., polyethylene glycol), cycloakyl, aryl, heterocycloalkyl, heteroaryl, carbohydrate, glycan, peptidoglycan, polypeptide, or any combination thereof. In some embodiments, a spacer comprises any one or more of the following units: an amide, ester, urea, carbonate, carbamate, disulfide, amino acid, amine, ether, alkyl, alkene, alkyne, heteroalkyl (e.g., PEG), cycloakyl, aryl, heterocycloalkyl, heteroaryl, carbohydrate, glycan, peptidoglycan, polypeptide, or any combination thereof. In some embodiments, a spacer L or L′ comprises a solubility enhancer or PK/PD modulator W. In some embodiments, a spacer comprises a glycosylated amino acid. In some embodiments, a spacer comprises one or more monosaccharide, disaccharide, polysaccharide, glycan, or peptidoglycan. In some embodiments, a spacer comprises a releasable moiety (e.g., a disulfide bond, an ester, or other moieties that can be cleaved in vivo). In some embodiments, a spacer comprises one or more units such as ethylene (e.g., polyethylene), ethylene glycol (e.g., PEG), ethanolamine, ethylenediamine, and the like (e.g., propylene glycol, propanolamine, propylenediamine). In some embodiments, a spacer comprises an oligoethylene, PEG, alkyl chain, oligopeptide, polypeptide, rigid functionality, peptidoglycan, oligoproline, oligopiperidine, or any combination thereof. In some embodiments, a spacer comprises an oligoethylene glycol or a PEG. A spacer can comprise an oligoethylene glycol. In some embodiments, a spacer comprises a PEG. In some embodiments, a spacer comprises an oligopeptide or polypeptide. In some embodiments, a spacer comprises an oligopeptide. In some embodiments, a spacer comprises a polypeptide. In some embodiments, a spacer comprises a peptidoglycan. In some embodiments, a spacer does not comprise a glycan. In some embodiments, a spacer does not comprise a sugar. In some embodiments, a rigid functionality is an oligoproline or oligopiperidine. In some embodiments, a rigid functionality is an oligoproline. In some embodiments, a rigid functionality is an oligopiperidine. In some embodiments, a rigid functionality is an oligophenyl. In some embodiments, a rigid functionality is an oligoalkyne. In some embodiments, an oligoproline or oligopiperidine has about two up to and including about fifty, about two to about forty, about two to about thirty, about two to about twenty, about two to about fifteen, about two to about ten, or about two to about six repeating units (e.g., prolines or piperidines).

In some embodiments, the compound comprises W groups to improve properties of the compound. In some embodiments, one or more G¹ are replaced with W, provided that one or more G¹ are not W. In some embodiments, one or more G¹ are replaced with W, provided that one or more G¹ are folate receptor ligands. In some embodiments, the linker L comprises one or more W groups. In some embodiments, W c comprises a solubility enhancer or PK/PD modulator. In some embodiments, W comprises polyethylene glycol (PEG), sugar, peptide, or peptidoglycan. In some embodiments, W comprises a PEG, sugar, peptide, or peptidoglycan for achieving better solubility and PK/PD properties. In some embodiments, W comprises one or more monosaccharide, disaccharide, peptide, peptidoglycan, and/or serum albumin. In some embodiments, W comprises one or more PEG, peptide, peptidoglycan, or serum albumin. In some embodiments, W does not comprise a sugar. In some embodiments, W does not comprise a monosaccharide, disaccharide, or polysaccharide. In some embodiments, W does not comprise a glycan. In some embodiments, W comprises a glycosylated amino acid. In some embodiments, W comprises a glycosylate cysteine. In some embodiments, W comprises a free carboxylic acid. In some embodiments, W comprises a PEG.

In some embodiments, W comprises one or more monosaccharide, disaccharide, oligosaccharide, polysaccharide, peptide, peptidoglycan, serum albumin, solubility enhancer, PK/PD modulator, or a combination thereof. In some embodiments, W modulates a pharmacological, pharmacokinetic, pharmacodynamic, or physicochemical property. In some embodiments, W facilitates internalization. In some embodiments, W improves aqueous solubility. In some embodiments, W increases plasma protein binding. In some embodiments, W modulates (e.g., reduces) the compound’s excretion, elimination, metabolism, stability (e.g., enzymatic stability, plasma stability), distribution, toxicity, or a combination thereof.

In some embodiments, a monosaccharide such as found in W exists in an equilibrium between its linear and cyclic form. In some embodiments, a monosaccharide is linear. In some embodiments, a monosaccharide is cyclic. In some embodiments, a monosaccharide exists as a D isomer. In some embodiments, a monosaccharide exists as an L isomer. As non-limiting examples, in some embodiments, W comprises one or more monosaccharides selected from the following: ribose, galactose, mannose, glucosefructose, N-acetylglucosamine, N-acetylmuramic acid or derivatives thereof (e.g., cyclic or linear forms, methylated derivatives, acetylated derivatives, phosphorylated derivatives, aminated derivatives, oxidized or reduced derivatives, D or L isomers, isotopes, stereoisomers, regioisomers, tautomers, or combinations thereof).

In some embodiments, a disaccharide, oligosaccharide, or polysaccharide, as may be disposed within W, contains an O-linkage, an N-linkage, a C-linkage, or a combination thereof. In some embodiments, a disaccharide, oligosaccharide, or polysaccharide contains a glycosidic linkage in either an alpha- or beta- orientation. In some embodiments, W comprises an oligosaccharide, a polysaccharide, or a glycan (e.g., a glycoprotein, glycopeptide, glycolipid, glycogen, proteoglycan, peptidoglycan, and the like).

In some embodiments, W comprises an amino acid, a peptide, a polypeptide, or a protein. In some embodiments, the amino acid is a natural amino acid (e.g., alanine (Ala), arginine (Arg), asparagine (Asn), aspartic acid (Asp), cysteine (Cys), glutamic acid (Glu), glutamine (Gln), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val)). Alternatively, in some embodiments, the amino acid is an unnatural or modified amino acid. W can comprise a sugar or sugar derivative covalently attached to the side chain of an amino acid (e.g., a glutamic acid, an aspartic acid).

In some embodiments, W comprises a glycosylated amino acid such as:

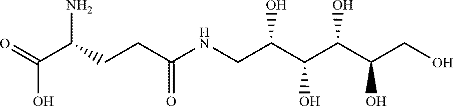

In some embodiments, a peptide or polypeptide comprises a plurality of amino acids, natural and/or unnatural. In some embodiments, a peptide (or peptidoglycan) has about two and about twenty amino acids. In some embodiments, an amino acid, a peptide, a polypeptide, or a protein (e.g., such as disposed within or making up W) has a pharmacological or physicochemical effect that enhances one or more properties of the compound (e.g., modulating solubility, solubility, size, permeability, protein binding, target binding, excretion, metabolism, toxicity, distribution, half-life, and/or duration of action). In some embodiments, W is a pharmacokinetic modulator. In some embodiments, the pharmacokinetic modulator is a peptide or protein that can modulate (e.g., enhance) protein binding. In some embodiments, the pharmacokinetic modulator enhances plasma protein binding. In some embodiments, the pharmacokinetic modulator reduces the rate of elimination, excretion, or metabolism. In some embodiments, the pharmacokinetic modulator increases the duration of action of the compound.

In some embodiments, the linker comprises an albumin ligand. In some embodiments, the albumin ligand comprises

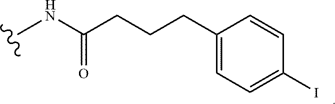

In some embodiments, the linker comprises a dimethylcysteine group. In some embodiments, the dimethylcysteine group is linked to a succinimide to form:

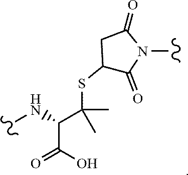

Compounds of formula (I) include compounds of the formulae:

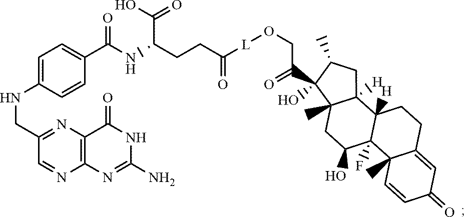

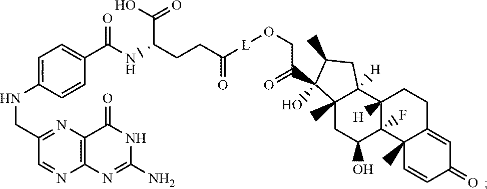

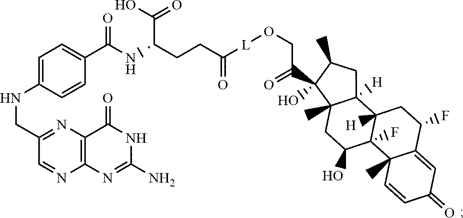

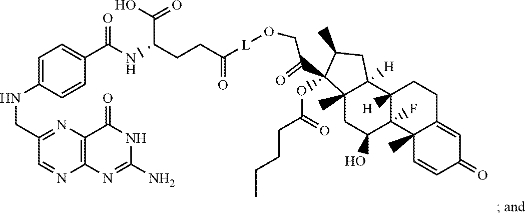

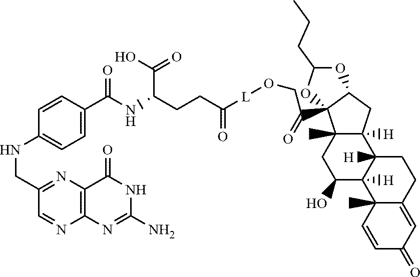

or a pharmaceutically acceptable salt, polymorph, prodrug, solvate or clathrate thereof, wherein the steroid is dexamethasone, flumethasone, betamethasone, betamethasone-17-valerate, and budesonide, respectively. Although the steroids are shown attached to L via the hydroxyl group α to the carbonyl of the D-ring, the connection of the steroids described and contemplated herein can be from any suitable portion of the steroid. Further, those of ordinary skill in the art will recognize that the folate targeting ligand and the steroid portions of the compounds of the formula (I) contain chiral centers. All diastereomers of the compounds described herein are contemplated, as well as racemates.

### Representative Compounds

Compounds of formula (I) include compounds of the formulae: Compound 101: [symbol] Compound 102: [symbol] Compound 103: [symbol] Compound 104: [symbol] Compound 105: [symbol] Compound 106 (Folate-PEG2-Hydrolink-DimethylCysteine-Carbamate-Dexamethasone): 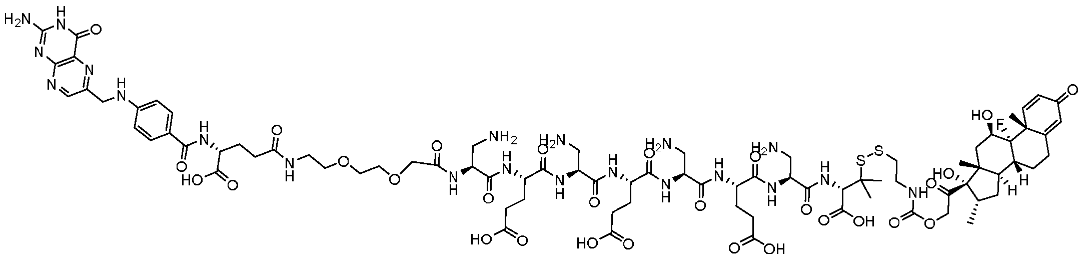 Compound 107 (Folate-AlbuminBinder-PEG2-Hydrolink-DimethylCysteine-Carbamate-Dexamethasone): 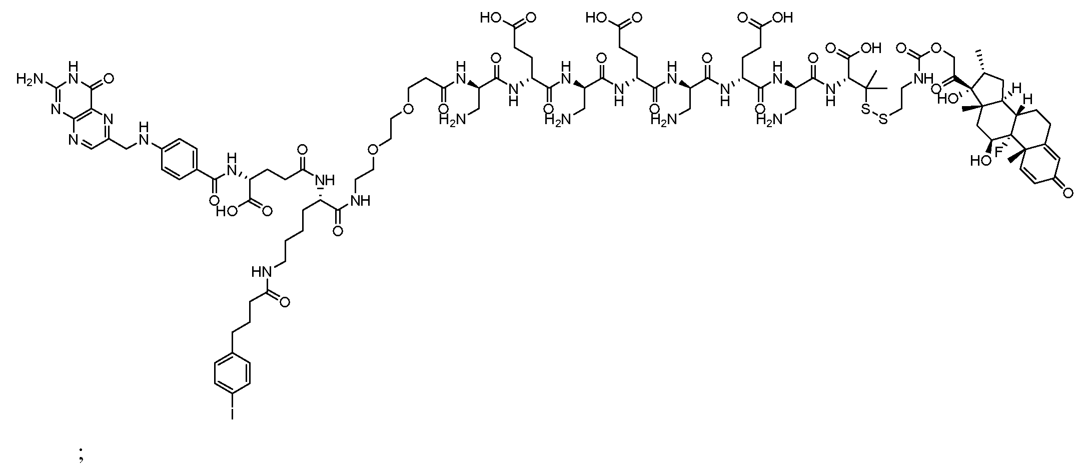 Compound 108 (Folate-PEG12(CH₂)-Pyrophosphate-Dexamethasone):

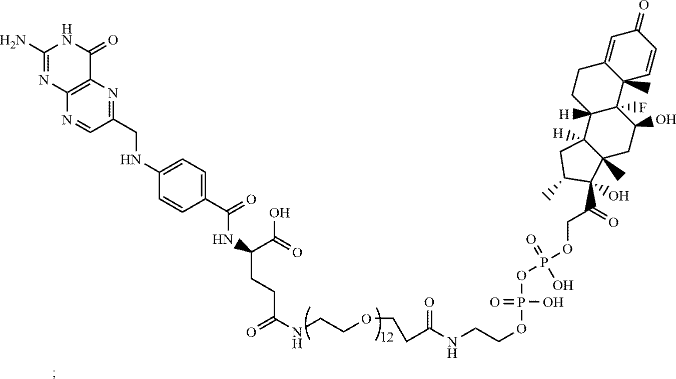 [SMILES: *C(=O)CCC(NC(=O)c1ccc(NCC2=NC3C(=O)NC(N)=NC3N=C2)cc1)C(=O)O]

Compound 109 (Folate-AlbuminBinder-PEG12(CH₂)-DimethylCysteine-Carbamate-Dexamethasone): 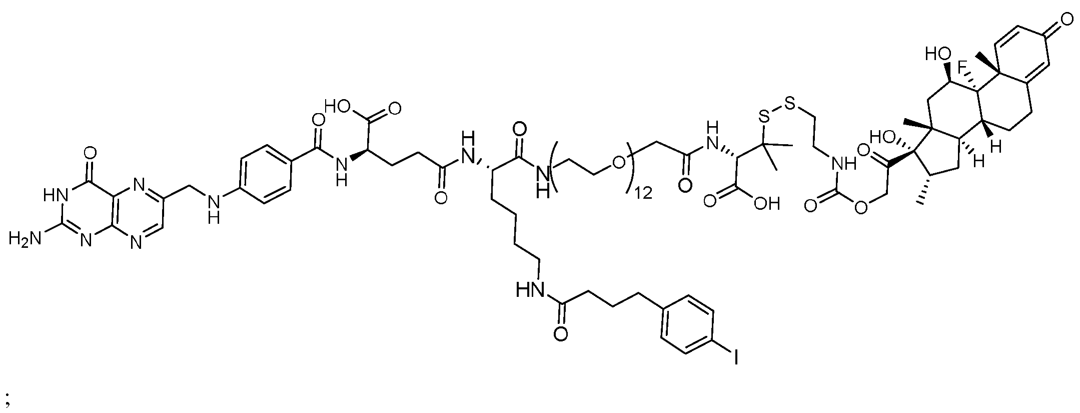 Compound 110 (Folate-AlbuminBinder-PEG12(CH₂)-DimethylCysteine-Carbamate-Dexamethasone): 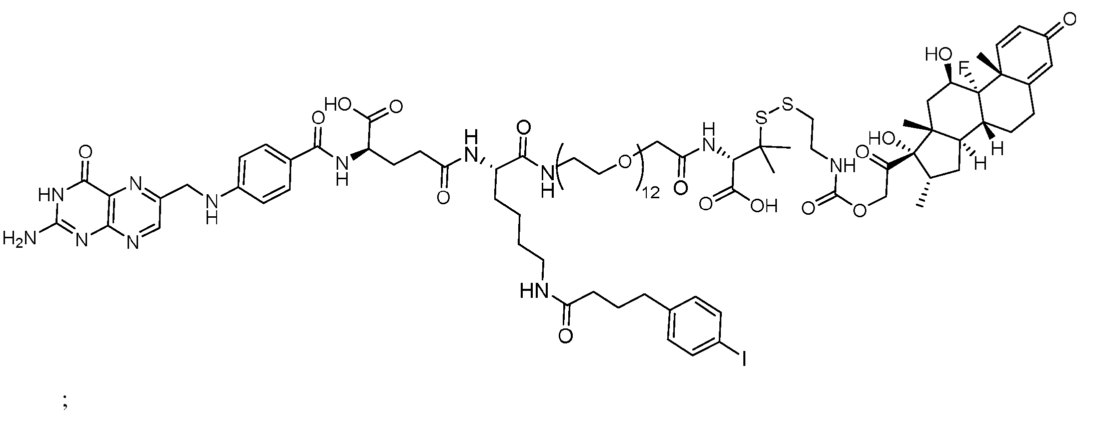 Compound 111 (Folate-PEG12(CH₂)-DimethylCysteine-Maleimide-CathepsinBCleavable-Dicarbamate-Dexamethasone): 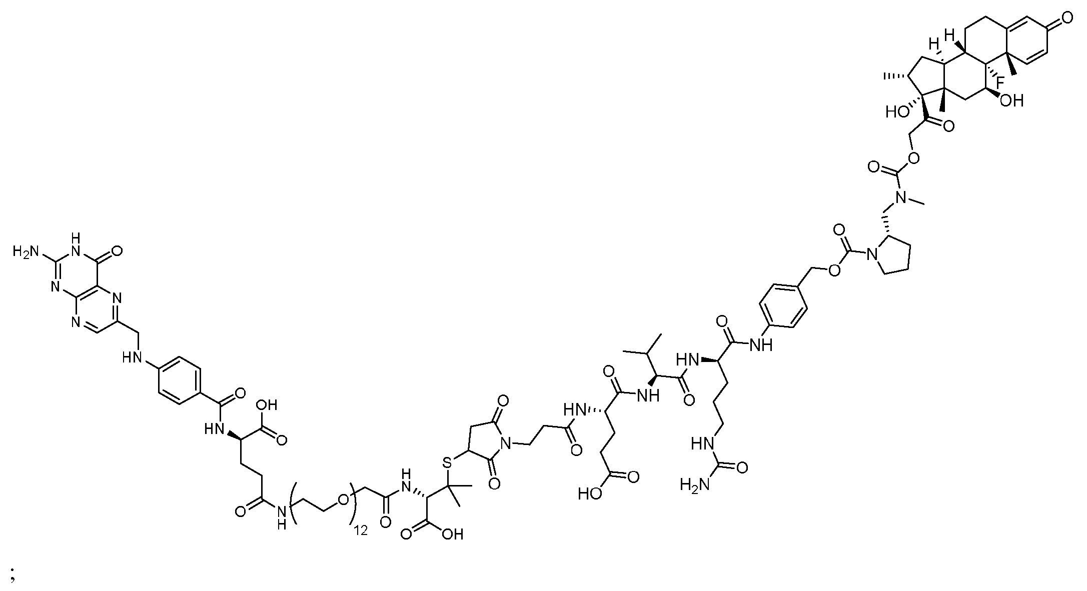 Compound 112 (Folate-AlbuminBinder-PEG12(CH₂)-DimethylCysteine-Maleimide-CathepsinBCleavable-Dicarbamate-Dexamethasone): 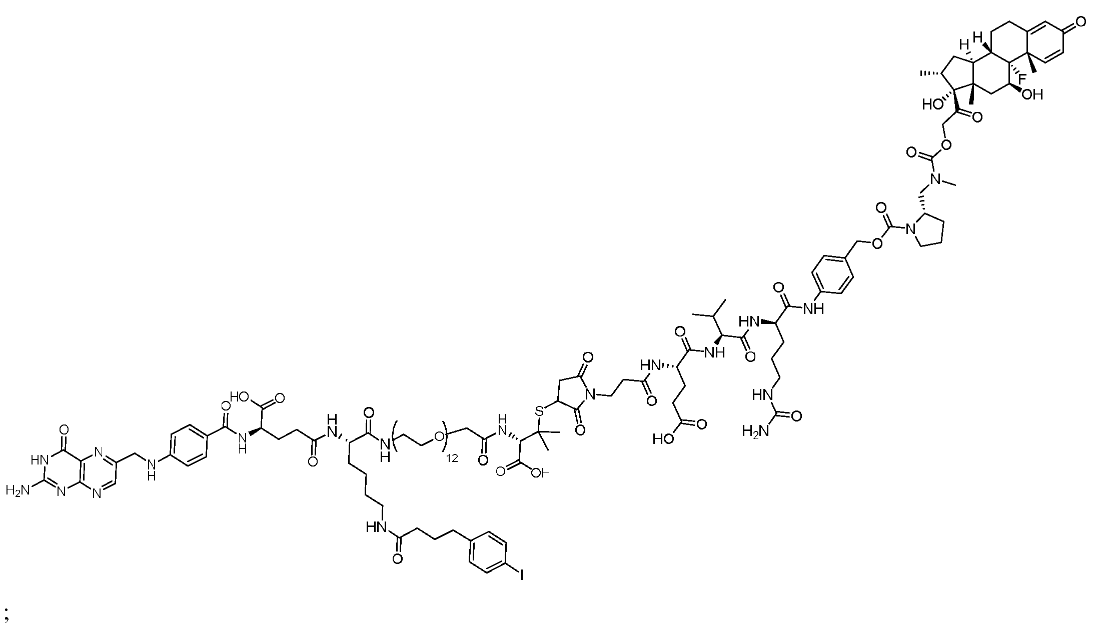 Compound 113 (Folate-AlbuminBinder-PEG2-Hydrolink-DimethylCysteine-Maleimide-CathepsinBCleavable-(CH₂CH₂)-DiCarbamate-Dexamethasone): 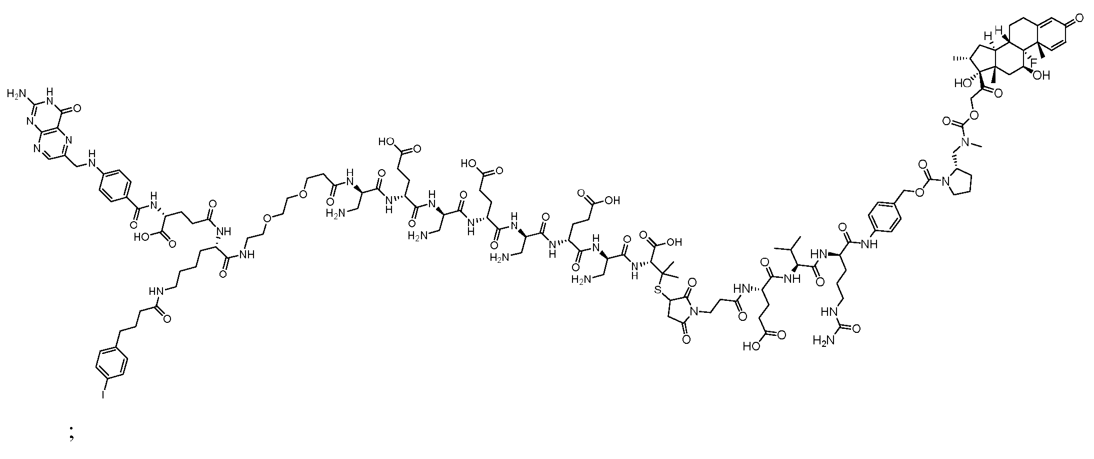 Compound 114 (Folate-AlbuminBinder-PEG2-Hydrolink-DimethylCysteine-Maleimide-CathepsinBCleavable-Phosphate-Dexamethasone): 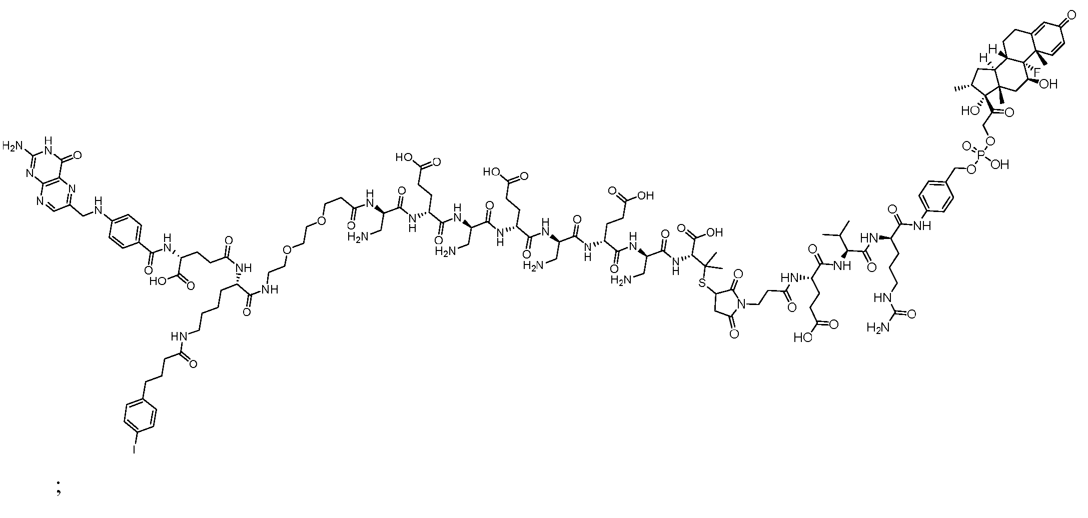 Compound 115 (Folate-AlbuminBinder-PEG2-Hydrolink-DimethylCysteine-Maleimide-BetaGlucuronidaseCleavable-Dicarbamate-Dexamethasone): 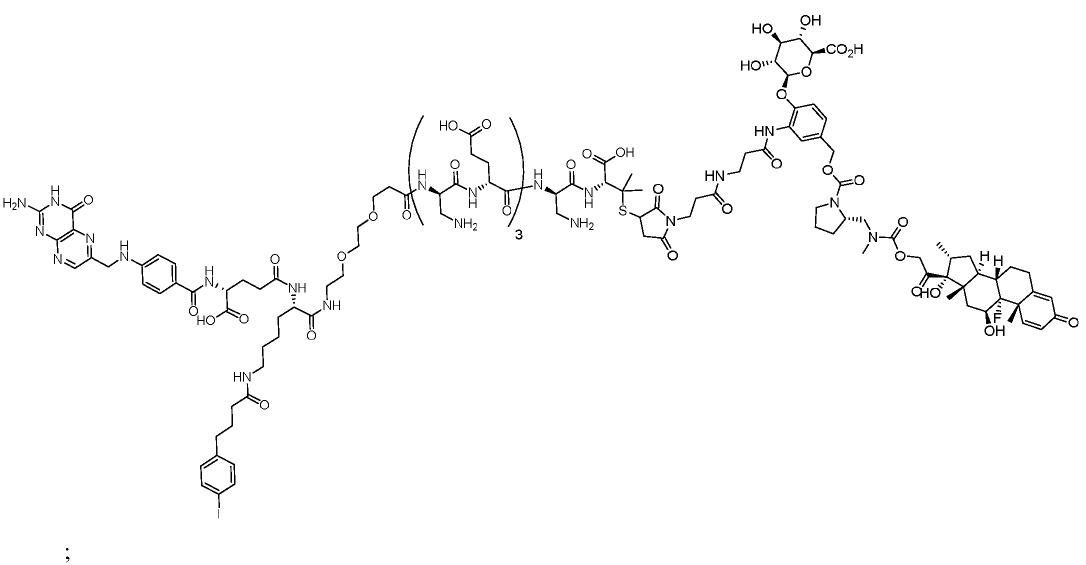 Compound 116 (Folate-AlbuminBinder-PEG12(CH₂)-DimethylCysteine-Maleimide-BetaGlucuronidaseCleavable-Dicarbamate-Dexamethasone): 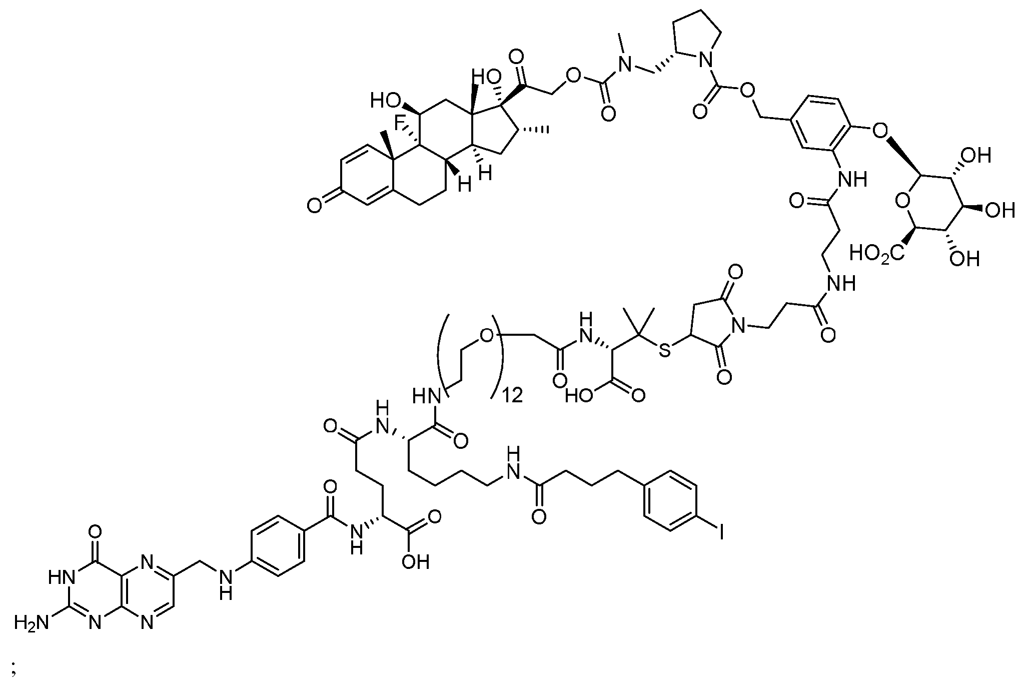 Compound 117 (Pteroyl-DimethylCysteine-CathepsinBCleavable-DiCarbamate-Dexamethasone): 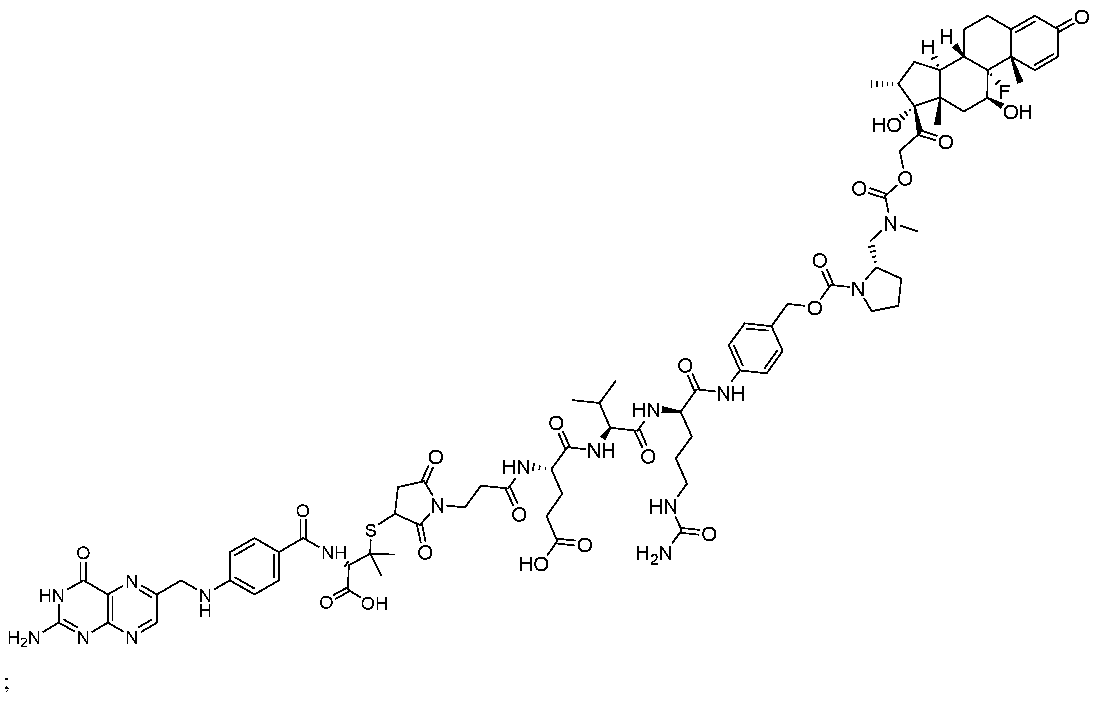 Compound 118 (Pteroyl-Lysine-Carbamate-Dexamethasone):

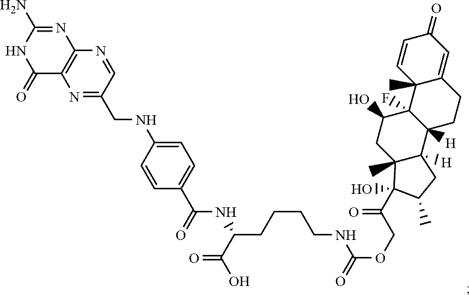

Compound 119 (Pteroyl-DimethylCysteine-Carbamate-Dexamethasone):

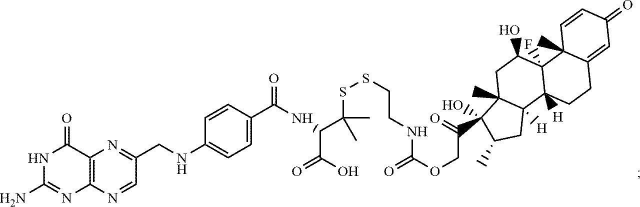

Compound 120 (Pteroyl-Serine-Phosphate-Dexamethasone):

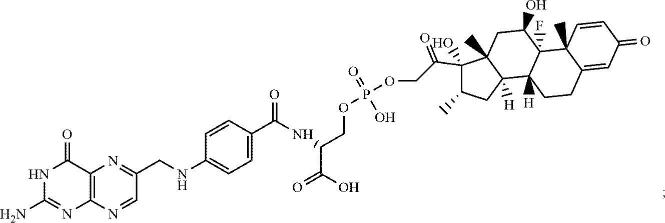

Compound 121 (Pteroyl-Serine-PyroPhosphate-Dexamethasone):

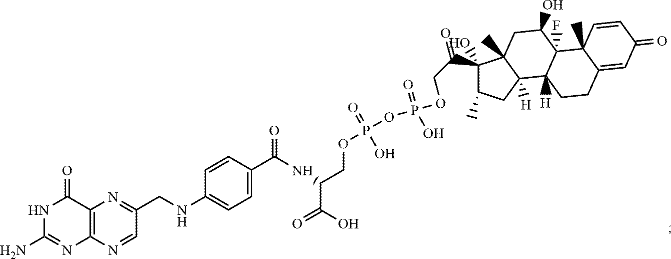

Compound 122 (Pteroyl-Threonine-Phosphate-Dexamethasone):

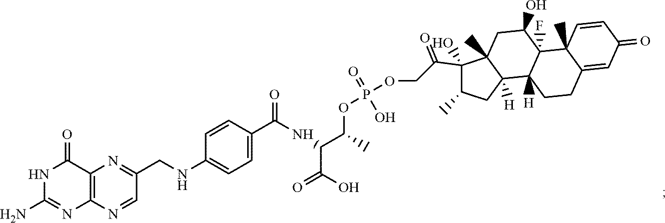

Compound 123 (Pteroyl-Threonine-PyroPhosphate-Dexamethasone):

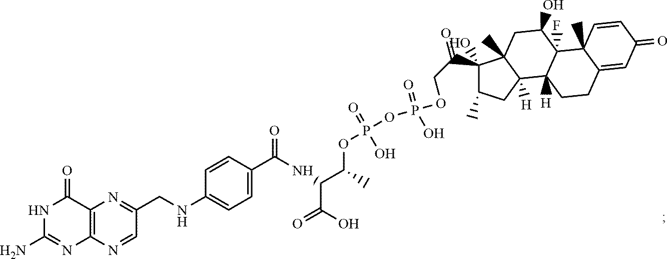

Compound 124 (Folate-AlbuminBinder-PEG2-Hydrolink-DimethylCysteine-Carbamate-Flumethasone) 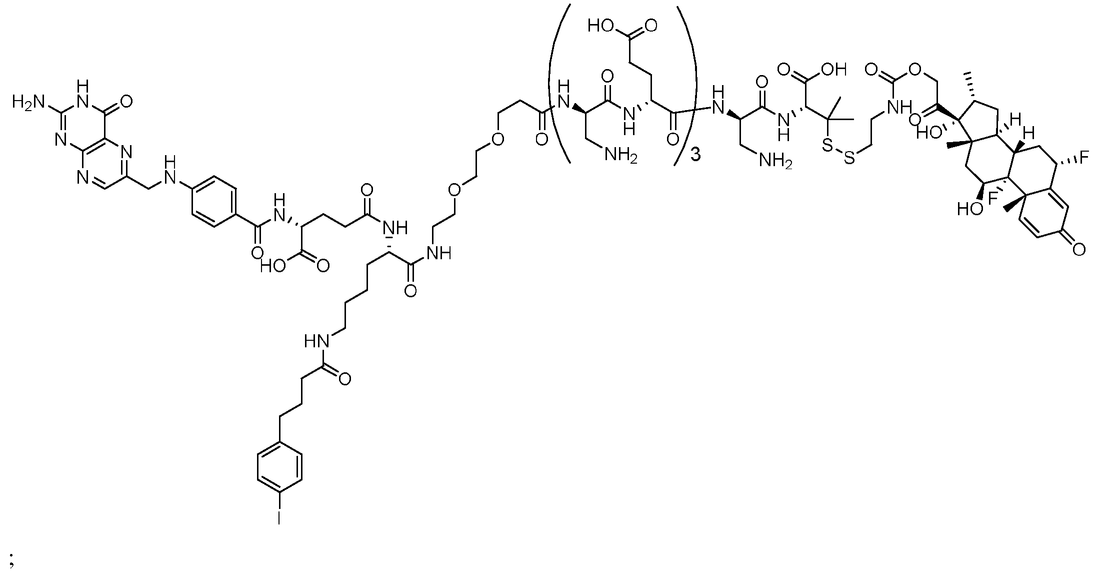 Compound 125 (Fol-Alb-PEG2-Hydrolink-DimethylCysteine-Maleimide-(CH2)₅-CathepsinB-Cleavable-Dicarbamate-Dexamethasone): 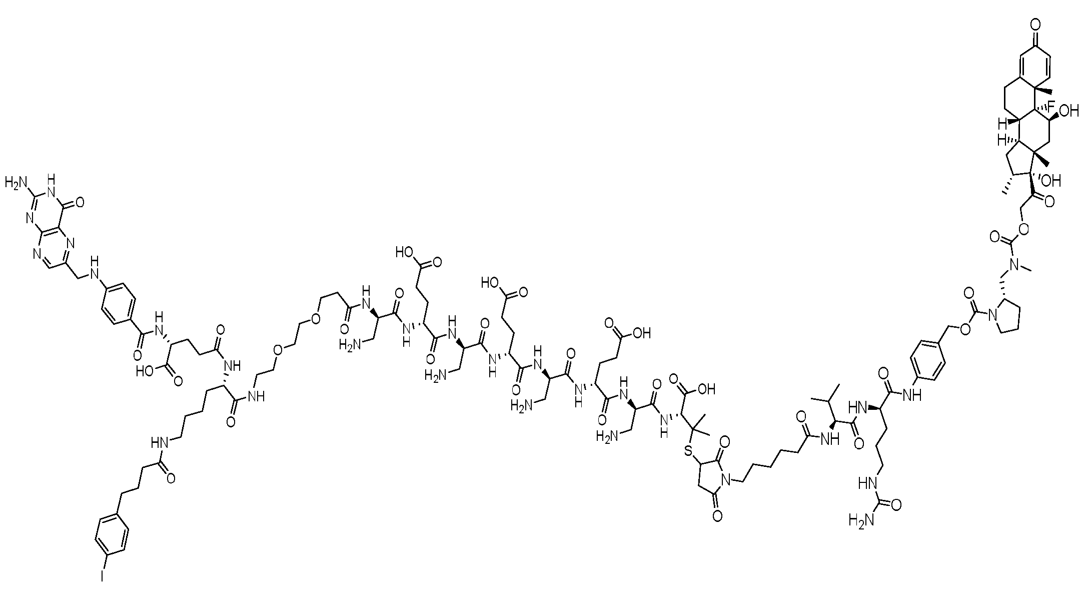 Compound 126 (Folate-AlbuminBinder-PEG2-Hydrolink-DimethylCysteine-Carbamate-Betamethasone) 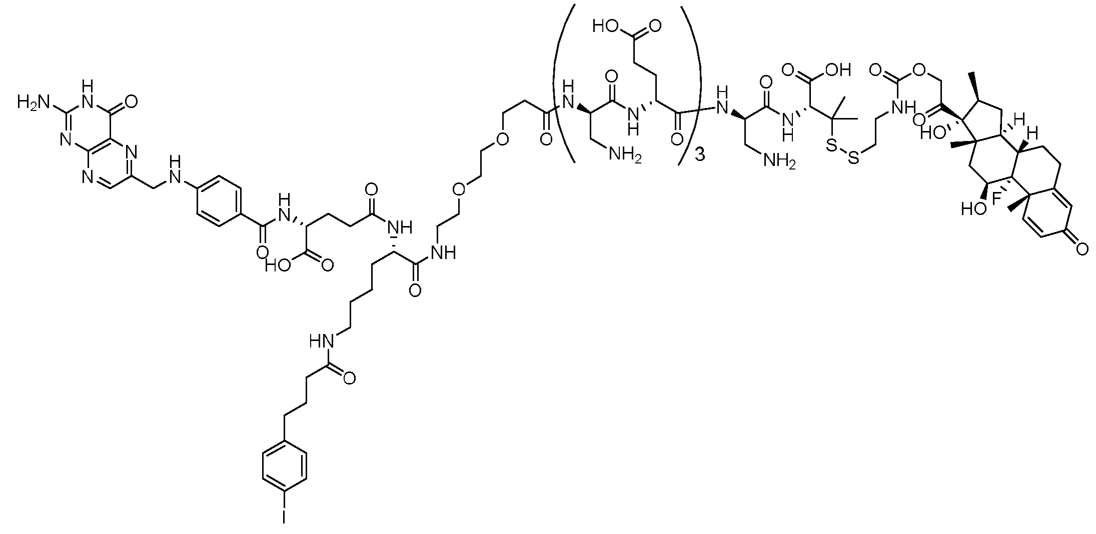 ; and Compound 127 (Folate-AlbuminBinder-PEG2-Hydrolink-DimethylCysteine-Carbamate-Budesonide) 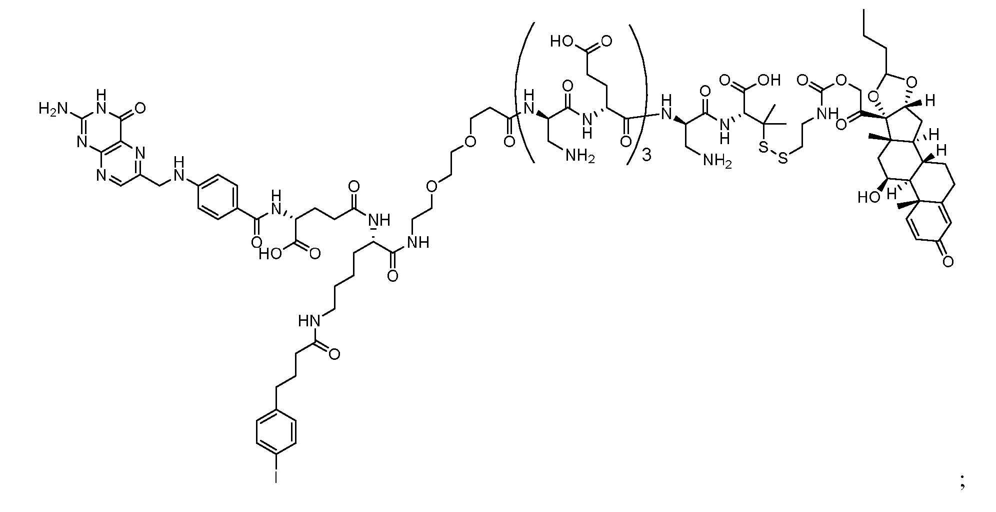 or a pharmaceutically acceptable salt, polymorph, prodrug, solvate or clathrate thereof

### Pharmaceutical Compositions

Disclosed herein, in certain embodiments, are pharmaceutical compositions comprising one or more compounds as described herein (e.g., a compound of the formula (I)) and one or more pharmaceutically acceptable excipients. Excipients are substances added to a pharmaceutical formulation which are not active ingredients., The class of excipients includes diluents (e.g., fillers used to, among other things, increase weight and improve content uniformity in tablets, including starches, hydrolyzed starches, partially pregelatinized starches; other examples of diluents include anhydrous lactose, lactose monohydrate, and sugar alcohols such as sorbitol, xylitol and mannitol). Such compositions may be specifically formulated for administration via one or more of a number of routes including, but not limited to, buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can be by means of capsule, drops, foams, gel, gum, injection, liquid, patch, pill, porous pouch, powder, tablet, or other suitable means of administration.

Also contemplated herein are pharmaceutical compositions comprising any compound described herein and at least one pharmaceutically acceptable excipient that is part of a nanoparticle, a liposomal or an exosomal formulation.

Pharmaceutical excipients generally do not provide any pharmacological activity to the formulation, though they provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

Any suitable pharmaceutically acceptable excipient may be used in the compositions disclosed herein. Suitable excipients include any and all solvents, dispersion media, coatings, antibacterial and antifungal agents, isotonic and absorption delaying agents that are physiologically compatible.

In some embodiments, the pharmaceutical composition is suitable for parenteral administration. Alternatively, the pharmaceutical composition is suitable for intravenous, intraperitoneal, intramuscular, sublingual, or oral administration. In some embodiments, the pharmaceutical composition is a sterile aqueous solution or dispersion or a sterile powder for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions disclosed herein is contemplated. Supplementary active compounds can also be incorporated into the compositions.

Pharmaceutical compositions may be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In some cases, isotonic agents can be included in the pharmaceutical compositions, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds described herein can be formulated in a time-release formulation, for example in a composition that includes a slow-release polymer. The active compounds can be prepared with carriers that will protect the compound against rapid release, such as a controlled-release formulation, including implants and microencapsulated delivery systems. Biodegradable, biocompatible polymers may be used, such as ethylene vinyl acetate, polyanhydrides, polyglycolic acid, collagen, polyorthoesters, polylactic acid and polylactic, and polyglycolic copolymers (PLG). Many methods for the preparation of such formulations are known to those skilled in the art.

Oral forms of administration are also contemplated herein. The pharmaceutical compositions may be orally administered as a capsule (hard or soft), tablet (film-coated, enteric-coated or uncoated), powder or granules (coated or uncoated) or liquid (solution or suspension). The formulations may be conveniently prepared by any of the methods well-known in the art. The pharmaceutical compositions may include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically- acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds, which can be included by admixture, are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a composition according to various embodiments may vary according to factors such as the disease state, age, gender, weight, patient history, risk factors, predisposition to disease, administration route, and pre-existing treatment regime (e.g., possible interactions with other medications). Dosage regimens may be adjusted to provide the optimum response. For example, a single bolus may be administered, several divided doses may be administered over time, or the dose may be proportionally reduced or increased as indicated by the exigencies of the situation. Desirably, a therapeutically effective amount is provided, and the amount is sufficient to provide a therapeutic effect to a subject having inflammation, such as inflammation associated with a disease or a disorder, or a prophylactic effect to a subject at risk for developing inflammation associated with a disease or disorder.

“Dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms for use with the methods disclosed herein are dictated by and directly dependent on the unique characteristics of the active compound and the particular therapeutic effect to be achieved, and the limitations inherent in the art of compounding such an active compound for the treatment of sensitivity in individuals. In therapeutic use for treatment of conditions in mammals (e.g., humans) for which the compounds of the various embodiments described herein, or an appropriate pharmaceutical composition thereof are effective, the compounds of the various embodiments described herein may be administered in an effective amount.

The dosage can be administered once, twice, or thrice a day, although more frequent dosing intervals are possible. The dosage may be administered every day, every 2 days, every 3 days, every 4 days, every 5 days, every 6 days, and/or every 7 days (once a week). In one embodiment, the dosage may be administered daily for up to and including 30 days, preferably between 7-10 days. In another embodiment, the dosage may be administered twice a day for 10 days. If the patient requires treatment for a chronic disease or condition, the dosage may be administered for as long as signs and/or symptoms persist. The patient may require “maintenance treatment” where the patient is receiving dosages every day for months, years, or for life. In addition, the compositions disclosed herein may be to effect prophylaxis of recurring symptoms. For example, the dosage may be administered once or twice a day to prevent the onset of symptoms in patients at risk, especially for asymptomatic patients.

The compositions described herein may be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to the cell, tissue, organ, and/or organ system, which encompasses and/or is affected by the disease, and/or where the disease signs and/or symptoms are active or are likely to occur. Administration can be topical with a local effect; composition is applied directly where its action is desired. Administration can be enteral wherein the desired effect is systemic (nonlocal), composition is given via the digestive tract. Administration can be parenteral, where the desired effect is systemic, composition is given by other routes than the digestive tract.

Compositions comprising a therapeutically effective amount of one or more compounds of the various embodiments described herein (e.g. a compound of the formula (I)) are also contemplated. The compositions are useful in a method for treating inflammation in a subject, the method comprising administering a therapeutically effective amount of a compound described herein to the subject. The therapeutically effective amount can provide a prophylactic or therapeutic effect. The inflammation can be treated with reduced, or elimination of, adverse effects relative to when the steroid is administered without the linkage to the folate targeting ligand. Adverse effects of steroids, such as those that are administered systemically include sodium (salt) and fluid retention, weight gain, edema, high blood pressure, muscle weakness, slow wound healing, and the like.

### Methods of Treatment

Disclosed herein, in certain embodiments are methods of treating inflammation in an individual in need thereof, comprising administering to the individual a therapeutically effective amount of one or more compounds disclosed herein or a pharmaceutical composition disclosed herein.

In some embodiments, the inflammation is associated with Crohn’s disease, lupus, inflammatory bowel disease (IBS), Addison’s disease, Grave’s disease, Sjogren’s syndrome, celiac disease, Hashimoto’s thyroiditis, myasthenia gravis, autoimmune vasculitis, reactive arthritis, psoriatic arthritis, pernicious anemia, ulcerative colitis, rheumatoid arthritis, type 1 diabetes, multiple sclerosis, or fibrotic disease, graft vs. host disease (GVHD), fatty liver disease, asthma, osteoporosis, sarcoidosis, ischemia-reperfusion injury, prosthesis osteolysis, glomerulonephritis, scleroderma, psoriasis, autoimmune myocarditis, spinal cord injury, central nervous system, viral infection, influenza, coronavirus infection, cytokine storm syndrome, bone damage, inflammatory brain disease, atherosclerosis, cancer, or a tumor.

Disclosed herein, in certain embodiments are methods of shifting macrophages from M1 to M2 in an individual in need thereof comprising administering to the individual a therapeutically effective amount of one or more compounds disclosed herein or a pharmaceutical composition disclosed herein.

Disclosed herein, in certain embodiments are methods for treating a disease or disorder that involves polarizing macrophages from M1 to M2 in a subject in need thereof comprising administering to the subject a therapeutically effective amount of a compound disclosed herein or a pharmaceutical composition disclosed herein. M1 and M2 macrophage profiles can be used to determine dosage, monitor efficacy of treatment (including therapeutic and prophylactic), and adjust dosage treatment plans. CD86 can be assessed to determine M1 macrophage levels, whereas CD206 can be assessed to determine M2 macrophage levels.

Disclosed herein, in certain embodiments are methods for treating a disease or disorder that involves polarizing tissue-resident macrophages (TRMs) known by other names from M1 to M2 in a subject in need thereof, comprising administering to the subject a therapeutically effective amount of a compound disclosed herein or a pharmaceutical composition disclosed herein. Those of skill in the art will recognize that depending on in which organs they reside, some TRMs have specific names, such as alveolar macrophages (AMs) (lung), microglia (brain), Kupffer cells (liver), renal macrophages (kidney), and osteoclasts (skeletal system).

Disclosed herein, in certain embodiments are methods for treating an inflammatory disorder in a subject in need thereof, comprising administering to the subject a therapeutically effective amount of a compound disclosed herein or a pharmaceutical composition described herein. In some embodiments, the inflammatory disorder is Crohn’s disease, lupus, inflammatory bowel disease (IBS), Addison’s disease, Grave’s disease, Sjogren’s syndrome, celiac disease, Hashimoto’s thyroiditis, myasthenia gravis, autoimmune vasculitis, reactive arthritis, psoriatic arthritis, pernicious anemia, ulcerative colitis, rheumatoid arthritis, type 1 diabetes, multiple sclerosis, or fibrotic disease, graft vs. host disease (GVHD), fatty liver disease, asthma, osteoporosis, sarcoidosis, ischemia-reperfusion injury, prosthesis osteolysis, glomerulonephritis, scleroderma, psoriasis, with autoimmune myocarditis, spinal cord injury, central nervous system, viral infection, influenza, coronavirus infection, cytokine storm syndrome, bone damage, inflammatory brain disease, atherosclerosis, cancer, or a tumor.

Disclosed herein, in certain embodiments are methods for treating inflammation associated with an autoimmune disease in a subject in need thereof, comprising administering to the subject a therapeutically effective amount of a compound disclosed herein or a pharmaceutical composition comprising one or more compounds disclosed herein.

Disclosed herein, in certain embodiments are methods for treating inflammation associated with an autoimmune disease in a subject in need thereof, comprising administering to the subject a therapeutically effective amount of a compound disclosed herein or a pharmaceutical composition disclosed herein, wherein G² is a radical of a steroid selected from the group consisting of budesonide, fludrocortisone, beclomethasone, fluticasone, mometasone, ciclesonide, cortisone, cortivazol, hydrocortisone, 21-hydroxypregnenolone, meprednisone, dexamethasone, triamcinolone, betamethasone, prednisone, prednisolone, and methylprednisolone. In some embodiments, the autoimmune disease is Crohn’s disease, lupus, inflammatory bowel disease, Addison’s disease, Graves’s disease, Sjogren’s syndrome, celiac disease, Hashimoto’s thyroiditis, myasthenia gravis, autoimmune vasculitis, reactive arthritis, psoriatic arthritis, pernicious anemia, ulcerative colitis, rheumatoid arthritis, type 1 diabetes, multiple sclerosis, asthma, osteoporosis, sarcoidosis, glomerulonephritis, scleroderma, psoriasis, or autoimmune myocarditis.

Disclosed herein, in certain embodiments are methods for treating inflammation associated with Crohn’s disease in a subject in need thereof, comprising administering to the subject a therapeutically effective amount of a compound disclosed herein or a pharmaceutical composition disclosed herein.

Disclosed herein, in certain embodiments are methods for treating inflammation associated with Crohn’s disease in a subject in need thereof, comprising administering to the subject a therapeutically effective amount of a compound disclosed herein or a pharmaceutical composition disclosed herein, wherein G² is a radical of a steroid selected from the group consisting of dexamethasone, hydrocortisone, budesonide, betamethasone, prednisone, prednisolone, and methylprednisolone.

Disclosed herein, in certain embodiments are methods for treating inflammation associated with lupus in a subject in need thereof, comprising administering to the subject a therapeutically effective amount of a compound disclosed herein or a pharmaceutical composition disclosed herein.

Disclosed herein, in certain embodiments are methods for treating inflammation associated with lupus in a subject in need thereof, comprising administering to the subject a therapeutically effective amount of a compound disclosed herein or a pharmaceutical composition disclosed herein, wherein G² is a radical of a steroid selected from the group consisting of dexamethasone, hydrocortisone, betamethasone, budesonide, prednisone, prednisolone, and methylprednisolone.

Disclosed herein, in certain embodiments are methods for treating inflammation associated with inflammatory bowel disease in a subject in need thereof, comprising administering to the subject a therapeutically effective amount of a compound disclosed herein or a pharmaceutical composition disclosed herein.

Disclosed herein, in certain embodiments are methods for treating inflammation associated with inflammatory bowel disease in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, hydrocortisone, budesonide, prednisone, prednisolone, and methylprednisolone.

Disclosed herein, in certain embodiments are methods for treating inflammation associated with Addison’s disease in a subject in need thereof, comprising administering to the subject a therapeutically effective amount of a compound disclosed herein or a pharmaceutical composition disclosed herein.

Disclosed herein, in certain embodiments are methods for treating inflammation associated with Addison’s disease in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, budesonide, hydrocortisone, fludrocortisone, prednisone, prednisolone, and methylprednisolone.

Disclosed herein, in certain embodiments are methods for treating inflammation associated with Grave’s disease in a subject in need thereof, comprising administering to the subject a therapeutically effective amount of a compound disclosed herein or a pharmaceutical composition disclosed herein.

Disclosed herein, in certain embodiments are methods for treating inflammation associated with Grave’s disease in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with Sjogren’s syndrome in a subject in need thereof, comprising administering to the subject a therapeutically effective amount of a compound disclosed herein or a pharmaceutical composition disclosed herein.

Disclosed herein, in certain embodiments are methods for treating inflammation associated with Sjogren’s syndrome in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with celiac disease in a subject in need thereof, comprising administering to the subject a therapeutically effective amount of a compound disclosed herein or a pharmaceutical composition disclosed herein.

Disclosed herein, in certain embodiments are methods for treating inflammation associated with celiac disease in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with Hashimoto’s thyroiditis in a subject in need thereof, comprising administering to the subject a therapeutically effective amount of a compound disclosed herein or a pharmaceutical composition disclosed herein.

Disclosed herein, in certain embodiments are methods for treating inflammation associated with Hashimoto’s thyroiditis in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with myasthenia gravis in a subject in need thereof, comprising administering to the subject a therapeutically effective amount of a compound disclosed herein or a pharmaceutical composition disclosed herein.

Disclosed herein, in certain embodiments are methods for treating inflammation associated with myasthenia gravis in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with autoimmune vasculitis in a subject in need thereof, comprising administering to the subject a therapeutically effective amount of a compound disclosed herein or a pharmaceutical composition disclosed herein.

Disclosed herein, in certain embodiments are methods for treating inflammation associated with autoimmune vasculitis in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with reactive arthritis in a subject in need thereof, comprising administering to the subject a therapeutically effective amount of a compound disclosed herein or a pharmaceutical composition disclosed herein.

Disclosed herein, in certain embodiments are methods for treating inflammation associated with reactive arthritis in a subject in need thereof, comprising administering to the subject a therapeutically effective amount of a compound disclosed herein or a pharmaceutical composition disclosed herein, wherein G² is a radical of a steroid selected from the group consisting of cortisone, hydrocortisone, dexamethasone, betamethasone, budesonide, prednisone, prednisolone, and methylprednisolone.

Disclosed herein, in certain embodiments are methods for treating inflammation associated with psoriatic arthritis in a subject in need thereof, comprising administering to the subject a therapeutically effective amount of a compound disclosed herein or a pharmaceutical composition disclosed herein.

Disclosed herein, in certain embodiments are methods for treating inflammation associated with psoriatic arthritis in a subject in need thereof, comprising administering to the subject a therapeutically effective amount of a compound disclosed herein or a pharmaceutical composition disclosed herein, wherein G² is a radical of a steroid selected from the group consisting of cortisone, hydrocortisone, dexamethasone, betamethasone, budesonide, prednisone, prednisolone, and methylprednisolone.

Disclosed herein, in certain embodiments are methods for treating inflammation associated with pernicious anemia in a subject in need thereof, comprising administering to the subject a therapeutically effective amount of a compound disclosed herein or a pharmaceutical composition disclosed herein.

Disclosed herein, in certain embodiments are methods for treating inflammation associated with pernicious anemia in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with ulcerative colitis in a subject in need thereof, comprising administering to the subject a therapeutically effective amount of a compound disclosed herein or a pharmaceutical composition disclosed herein.

Disclosed herein, in certain embodiments are methods for treating inflammation associated with ulcerative colitis in a subject in need thereof, comprising administering to the subject a therapeutically effective amount of a compound disclosed herein or a pharmaceutical composition disclosed herein, wherein G² is a radical of a steroid selected from the group consisting of cortisone, hydrocortisone, triamcinolone, beclomethasone, betamethasone, dexamethasone, budesonide, prednisone, prednisolone, and methylprednisolone.

Disclosed herein, in certain embodiments are methods for treating inflammation associated with rheumatoid arthritis in a subject in need thereof, comprising administering to the subject a therapeutically effective amount of a compound disclosed herein or a pharmaceutical composition disclosed herein.

Disclosed herein, in certain embodiments are methods for treating inflammation associated with rheumatoid arthritis in a subject in need thereof, comprising administering to the subject a therapeutically effective amount of a compound disclosed herein or a pharmaceutical composition disclosed herein, wherein G² is a radical of a steroid selected from the group consisting of cortisone, hydrocortisone, triamcinolone, beclomethasone, betamethasone, dexamethasone, budesonide, prednisone, prednisolone, and methylprednisolone.

Disclosed herein, in certain embodiments are methods for treating inflammation associated with type 1 diabetes in a subject in need thereof, comprising administering to the subject a therapeutically effective amount of a compound disclosed herein, or a pharmaceutical composition disclosed herein.

Disclosed herein, in certain embodiments are methods for treating inflammation associated with type 1 diabetes in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with multiple sclerosis in a subject in need thereof, comprising administering to the subject a therapeutically effective amount of a compound disclosed herein or a pharmaceutical composition disclosed herein.

Disclosed herein, in certain embodiments are methods for treating inflammation associated with multiple sclerosis in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with asthma in a subject in need thereof, comprising administering to the subject a therapeutically effective amount of a compound disclosed herein, or a pharmaceutical composition disclosed herein.

Disclosed herein, in certain embodiments are methods for treating inflammation associated with asthma in a subject in need thereof, comprising administering to the subject a therapeutically effective amount of a compound disclosed herein, or a pharmaceutical composition disclosed herein, wherein G² is a radical of a steroid selected from the group consisting of triamcinolone, fluticasone, budesonide, mometasone, beclomethasone, ciclesonide, dexamethasone, betamethasone, budesonide, prednisone, prednisolone, and methylprednisolone.

Disclosed herein, in certain embodiments are methods for treating inflammation associated with osteoporosis in a subject in need thereof, comprising administering to the subject a therapeutically effective amount of a compound disclosed herein, or a pharmaceutical composition disclosed herein.

Disclosed herein, in certain embodiments are methods for treating inflammation associated with osteoporosis in a subject in need thereof, comprising administering to the subject a therapeutically effective amount of a compound disclosed herein, or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with sarcoidosis in a subject in need thereof, comprising administering to the subject a therapeutically effective amount of a compound disclosed herein, or a pharmaceutical composition disclosed herein.

Disclosed herein, in certain embodiments are methods for treating inflammation associated with sarcoidosis in a subject in need thereof, comprising administering to the subject a therapeutically effective amount of a compound disclosed herein, or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with glomerulonephritis in a subject in need thereof, comprising administering to the subject a therapeutically effective amount of a compound disclosed herein, or a pharmaceutical composition disclosed herein.

Disclosed herein, in certain embodiments are methods for treating inflammation associated with glomerulonephritis in a subject in need thereof, comprising administering to the subject a therapeutically effective amount of a compound disclosed herein, or a pharmaceutical composition disclosed herein, wherein G² is a radical of a steroid selected from the group consisting of dexamethasone, budesonide, betamethasone, prednisone, prednisolone, and methylprednisolone.

Disclosed herein, in certain embodiments are methods for treating inflammation associated with autoimmune myocarditis in a subject in need thereof, comprising administering to the subject a therapeutically effective amount of a compound disclosed herein, or a pharmaceutical composition disclosed herein.

Disclosed herein, in certain embodiments are methods for treating inflammation associated with autoimmune myocarditis in a subject in need thereof, comprising administering to the subject a therapeutically effective amount of a compound disclosed herein, or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with a fibrotic disease (e.g., a fibrotic disease that results from inflammation) in a subject in need thereof, comprising administering to the subject a therapeutically effective amount of a compound disclosed herein, or a pharmaceutical composition disclosed herein.

Disclosed herein, in certain embodiments are methods for treating inflammation associated with a fibrotic disease (e.g., a fibrotic disease that results from inflammation) in a subject in need thereof, comprising administering to the subject a therapeutically effective amount of a compound disclosed herein, or a pharmaceutical composition comprising one or more compounds disclosed herein, wherein G² is a radical of a steroid selected from the group consisting of dexamethasone, betamethasone, budesonide, and prednisone.

Disclosed herein, in certain embodiments are methods for treating inflammation associated with graft vs. host disease (GVHD), in a subject in need thereof, comprising administering to the subject a therapeutically effective amount of a compound disclosed herein or a pharmaceutical composition disclosed herein.

Disclosed herein, in certain embodiments are methods for treating inflammation associated with graft vs. host disease (GVHD), in a subject in need thereof, comprising administering to the subject a therapeutically effective amount of a compound disclosed herein, or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with fatty liver disease in a subject in need thereof, comprising administering to the subject a therapeutically effective amount of a compound disclosed herein, or a pharmaceutical composition disclosed herein.

Disclosed herein, in certain embodiments are methods for treating inflammation associated with fatty liver disease in a subject in need thereof, comprising administering to the subject a therapeutically effective amount of a compound disclosed herein, or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with ischemia-reperfusion injury in a subject in need thereof, comprising administering to the subject a therapeutically effective amount of a compound disclosed herein, or a pharmaceutical composition disclosed herein.

Disclosed herein, in certain embodiments are methods for treating inflammation associated with ischemia-reperfusion injury in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with prosthesis osteolysis in a subject in need thereof, comprising administering to the subject a therapeutically effective amount of a compound disclosed herein, or a pharmaceutical composition disclosed herein.

Disclosed herein, in certain embodiments are methods for treating inflammation associated with prosthesis osteolysis in a subject in need thereof, comprising administering to the subject a therapeutically effective amount of a compound disclosed herein, or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with scleroderma in a subject in need thereof, comprising administering to the subject a therapeutically effective amount of a compound disclosed herein, or a pharmaceutical composition comprising one or more compounds disclosed herein.

Disclosed herein, in certain embodiments are methods for treating inflammation associated with scleroderma in a subject in need thereof, comprising administering to the subject a therapeutically effective amount of a compound disclosed herein, or a pharmaceutical composition disclosed herein, wherein G² is a radical of a steroid selected from the group consisting of dexamethasone, budesonide, betamethasone, triamcinolone, prednisone, prednisolone, and methylprednisolone.

Disclosed herein, in certain embodiments are methods for treating inflammation associated with psoriasis in a subject in need thereof, comprising administering to the subject a therapeutically effective amount of a compound disclosed herein, or a pharmaceutical composition disclosed herein.

Disclosed herein, in certain embodiments are methods for treating inflammation associated with psoriasis in a subject in need thereof, comprising administering to the subject a therapeutically effective amount of a compound disclosed herein, or a pharmaceutical composition disclosed herein, wherein G² is a radical of a steroid selected from the group consisting of budesonide, betamethasone, triamcinolone, prednisone, prednisolone, methylprednisolone, hydrocortisone, dexamethasone, hydrocortisone-17-valerate, diflorasone, meprednisone, halobetacol, tixocortol, amcinonide, desonide, fluocinolone acetonide, fluocinonide, halcinonide, beclomethasone, and halometasone.

Disclosed herein, in certain embodiments are methods for treating inflammation associated with spinal cord injury in a subject in need thereof, comprising administering to the subject a therapeutically effective amount of a compound disclosed herein, or a pharmaceutical composition disclosed herein.

Disclosed herein, in certain embodiments are methods for treating inflammation associated with spinal cord injury in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation of the central nervous system in a subject in need thereof, comprising administering to the subject a therapeutically effective amount of a compound disclosed herein, or a pharmaceutical composition disclosed herein.

Disclosed herein, in certain embodiments are methods for treating inflammation of the central nervous system in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with a viral infection in a subject in need thereof, comprising administering to the subject a therapeutically effective amount of a compound disclosed herein, or a pharmaceutical composition disclosed herein.

Disclosed herein, in certain embodiments are methods for treating inflammation associated with a viral infection in a subject in need thereof, comprising administering to the subject a therapeutically effective amount of a compound disclosed herein or a pharmaceutical composition disclosed herein, wherein G² is a radical of a steroid selected from the group consisting of hydrocortisone, dexamethasone, betamethasone, budesonide, prednisone, prednisolone, and methylprednisolone.

Disclosed herein, in certain embodiments are methods for treating inflammation associated with influenza in a subject in need thereof, comprising administering to the subject a therapeutically effective amount of a compound disclosed herein, or a pharmaceutical composition disclosed herein.

Disclosed herein, in certain embodiments are methods for treating inflammation associated with influenza in a subject in need thereof, comprising administering to the subject a therapeutically effective amount of a compound disclosed herein or a pharmaceutical composition disclosed herein, wherein G² is a radical of a steroid selected from the group consisting of hydrocortisone, dexamethasone, betamethasone, budesonide, prednisone, prednisolone, and methylprednisolone.

Disclosed herein, in certain embodiments are methods for treating inflammation associated with SARS-CoV-2 (COVID-19) infection in a subject in need thereof, comprising administering to the subject a therapeutically effective amount of a compound disclosed herein or a pharmaceutical composition disclosed herein.

Disclosed herein, in certain embodiments are methods for treating inflammation associated with SARS-CoV-2 (COVID-19) infection in a subject in need thereof, comprising administering to the subject a therapeutically effective amount of a compound disclosed herein or a pharmaceutical composition disclosed herein, wherein G² is a radical of a steroid selected from the group consisting of hydrocortisone, dexamethasone, betamethasone, budesonide, and prednisone, prednisolone, and methylprednisolone.

Disclosed herein, in certain embodiments are methods for treating inflammation associated with cytokine storm syndrome in a subject in need thereof, comprising administering to the subject a therapeutically effective amount of a compound disclosed herein, or a pharmaceutical composition disclosed herein.

Disclosed herein, in certain embodiments are methods for treating inflammation associated with cytokine storm syndrome in a subject in need thereof, comprising administering to the subject a therapeutically effective amount of a compound disclosed herein or a pharmaceutical composition disclosed herein, wherein G² is a radical of a steroid selected from the group consisting of hydrocortisone, dexamethasone, betamethasone, budesonide, prednisone, prednisolone, and methylprednisolone.

Disclosed herein, in certain embodiments are methods for treating inflammation associated with damage to bone in a subject in need thereof, comprising administering to the subject a therapeutically effective amount of a compound disclosed herein or a pharmaceutical composition disclosed herein.

Disclosed herein, in certain embodiments are methods for treating inflammation associated with damage to bone in a subject in need thereof, comprising administering to the subject a therapeutically effective amount of a compound disclosed herein or a pharmaceutical composition disclosed herein, wherein G² is a radical of a steroid selected from the group consisting of cortisone, cortivazol, hydrocortisone, 21-hydroxypregnenolone, meprednisone, dexamethasone, triamcinolone, betamethasone, budesonide, prednisone, prednisolone, and methylprednisolone.

Disclosed herein, in certain embodiments are methods for treating inflammation associated with inflammatory brain disease in a subject in need thereof, comprising administering to the subject a therapeutically effective amount of a compound disclosed herein or a pharmaceutical composition disclosed herein.

Disclosed herein, in certain embodiments are methods for treating inflammation associated with inflammatory brain disease in a subject in need thereof, comprising administering to the subject a therapeutically effective amount of a compound disclosed herein or a pharmaceutical composition disclosed herein, wherein G² is a radical of a steroid selected from the group consisting of dexamethasone, betamethasone, budesonide, prednisone, prednisolone, and methylprednisolone.

Disclosed herein, in certain embodiments are methods for treating inflammation associated with atherosclerosis in a subject in need thereof, comprising administering to the subject a therapeutically effective amount of a compound disclosed herein or a pharmaceutical composition disclosed herein.

Disclosed herein, in certain embodiments are methods for treating inflammation associated with atherosclerosis in a subject in need thereof, comprising administering to the subject a therapeutically effective amount of a compound disclosed herein or a pharmaceutical composition disclosed herein, wherein G² is a radical of a steroid selected from the group consisting of fluticasone, budesonide, beclomethasone, ciclesonide, dexamethasone, betamethasone, prednisone, prednisolone, and methylprednisolone.

Disclosed herein, in certain embodiments are methods for treating inflammation associated with a tumor in a subject in need thereof, comprising administering to the subject a therapeutically effective amount of a compound disclosed herein, or a pharmaceutical composition disclosed herein.

Disclosed herein, in certain embodiments are methods for treating inflammation associated with a tumor in a subject in need thereof, comprising administering to the subject a therapeutically effective amount of a compound disclosed herein or a pharmaceutical composition disclosed herein, wherein G² is a radical of a steroid selected from the group consisting of cortisone, hydrocortisone, clobetasol, dexamethasone, betamethasone, budesonide, meprednisone, prednisone, prednisolone, and methylprednisolone.

Disclosed herein, in certain embodiments are methods for treating inflammation associated with cancer in a subject in need thereof, comprising administering to the subject a therapeutically effective amount of a compound disclosed herein, or a pharmaceutical composition disclosed herein.

Disclosed herein, in certain embodiments are methods for treating inflammation associated with cancer in a subject in need thereof, comprising administering to the subject a therapeutically effective amount of a compound disclosed herein or a pharmaceutical composition disclosed herein, wherein G² is a radical of a steroid selected from the group consisting of cortisone, hydrocortisone, clobetasol, dexamethasone, betamethasone, budesonide, meprednisone, prednisone, prednisolone, and methylprednisolone.

In some embodiments, the therapeutically effective amount is that which may treat or alleviate the disease, signs or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically effective dose level for any particular patient will depend upon a variety of factors, including the condition being treated and the severity of the condition; activity of the specific compound employed; the specific composition employed; the age, body weight, general health, gender and diet of the patient; the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable adverse effect, that might occur during administration of one or more of the compounds described herein.

It will be understood by one of ordinary skill in the relevant arts that other suitable modifications and adaptations to the compositions and methods described herein are readily apparent from the description of the disclosure contained herein in view of information known to the ordinarily skilled artisan, and may be made without departing from the scope of the disclosure. Having now described the present disclosure in detail, the same will be more clearly understood by reference to the following examples, which are included herewith for purposes of illustration only and are not intended to be limiting of the disclosure.

### EXAMPLES

The present disclosure can be better understood by reference to the following examples which are offered by way of illustration.

All LC-MS data was acquired on a 0-100% gradient method, utilizing a C18 column along with 20 mM ammonium bicarbonate pH 7 and acetonitrile.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

### Example 1

This example describes the synthesis of a compound comprising folate as a ligand, a linker, and dexamethasone as a steroid.

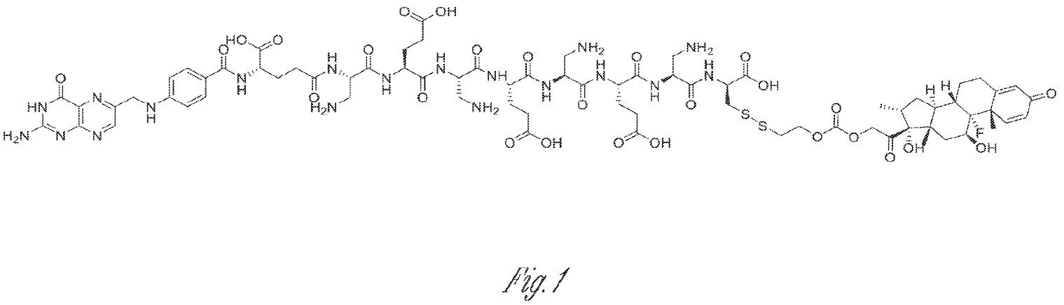

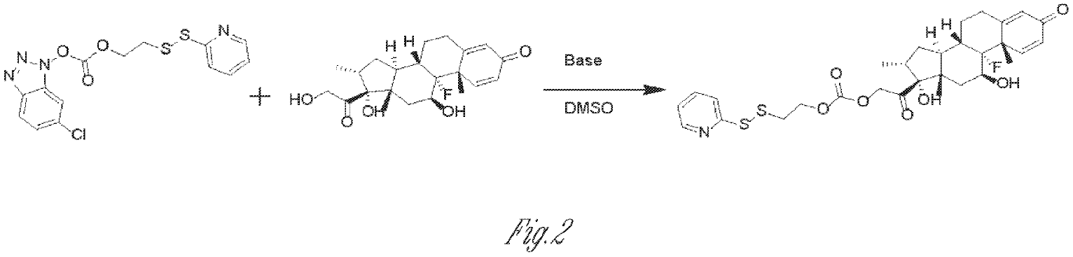

FIG. 1 shows the chemical structure of a “drug compound,” otherwise known as compound 101. The drug compound from left to right is composed of folate as the ligand. The folate ligand is covalently linked to a hydrophilic linker composed of alternating unnatural amino acid D-glutamic acid, diaminopropionic acid (Dap), and the natural amino acid cysteine. The cysteine is then connected via a disulfide self-immolative linker to dexamethasone. The structure shown is also named compound 101, or Folate-Hydrolink-Cysteine-Carbonate-Dexamethasone.

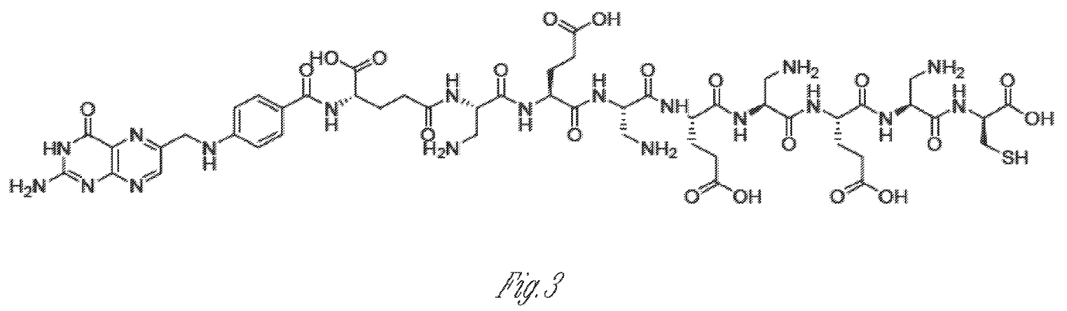

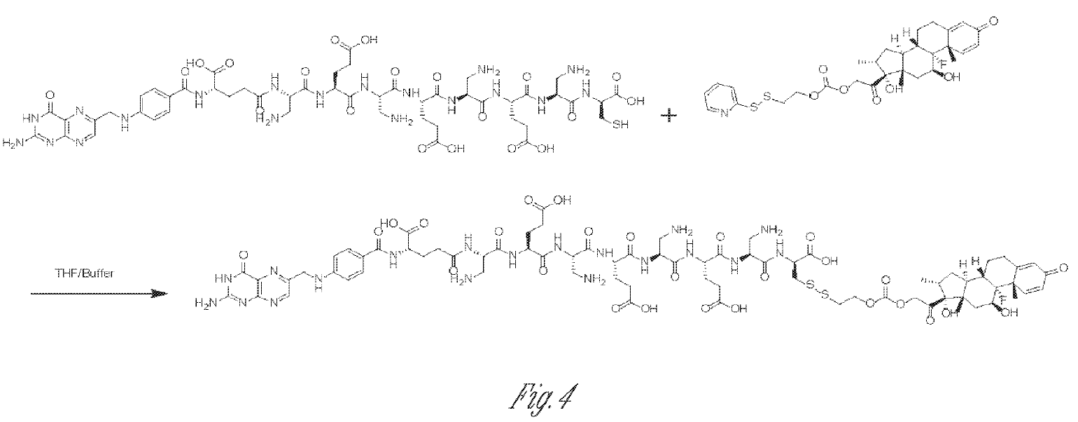

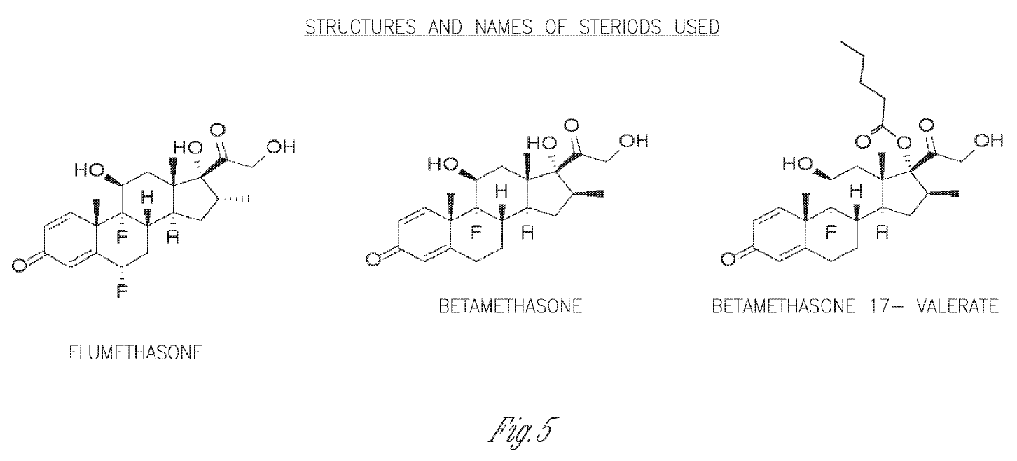

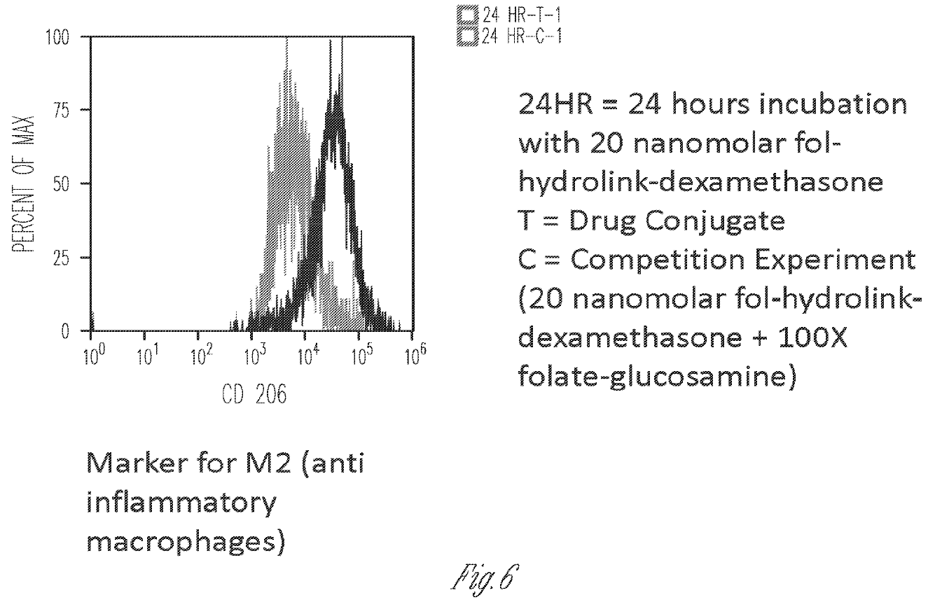

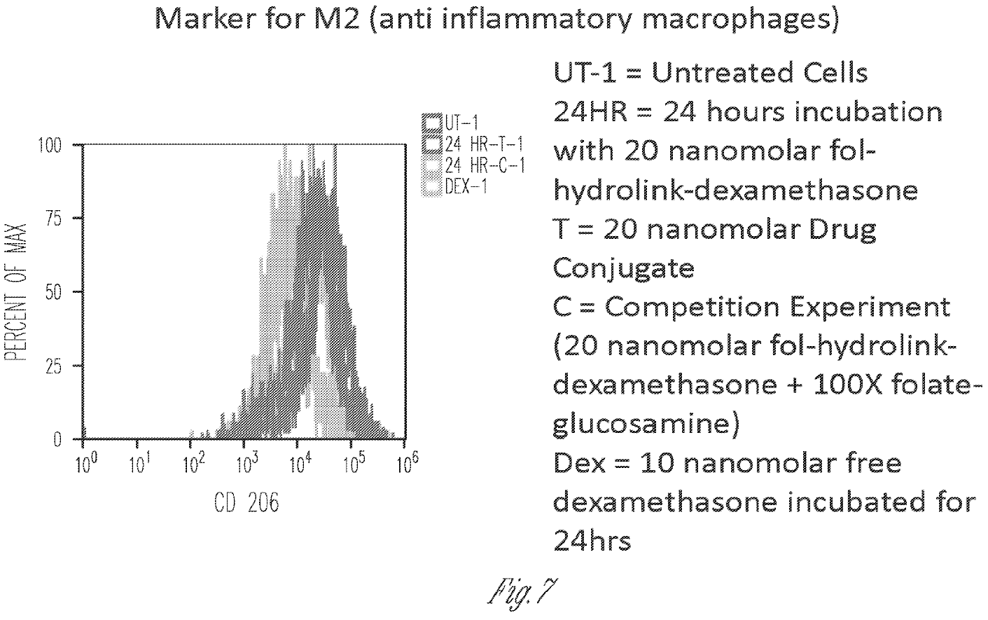

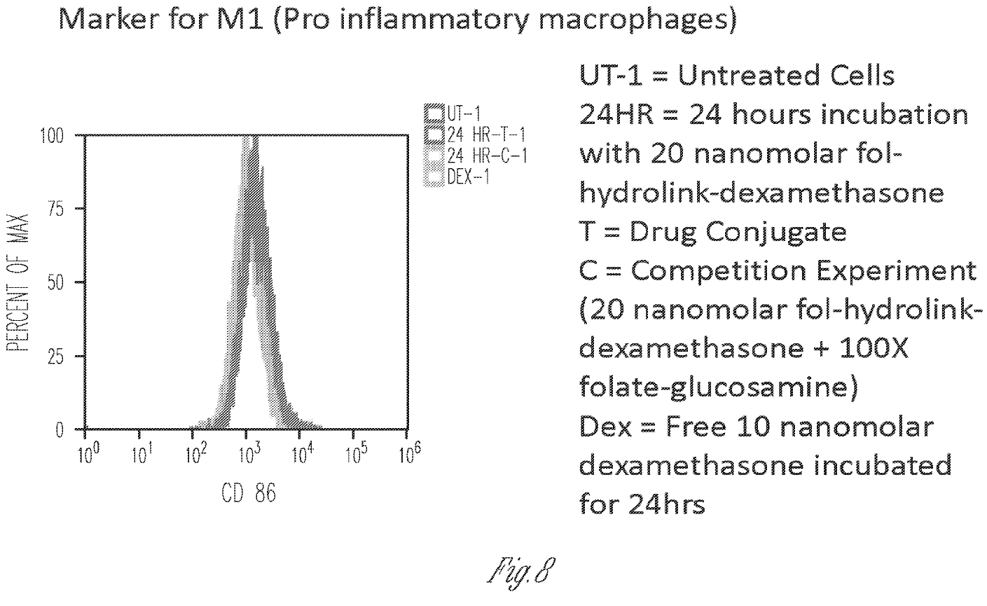

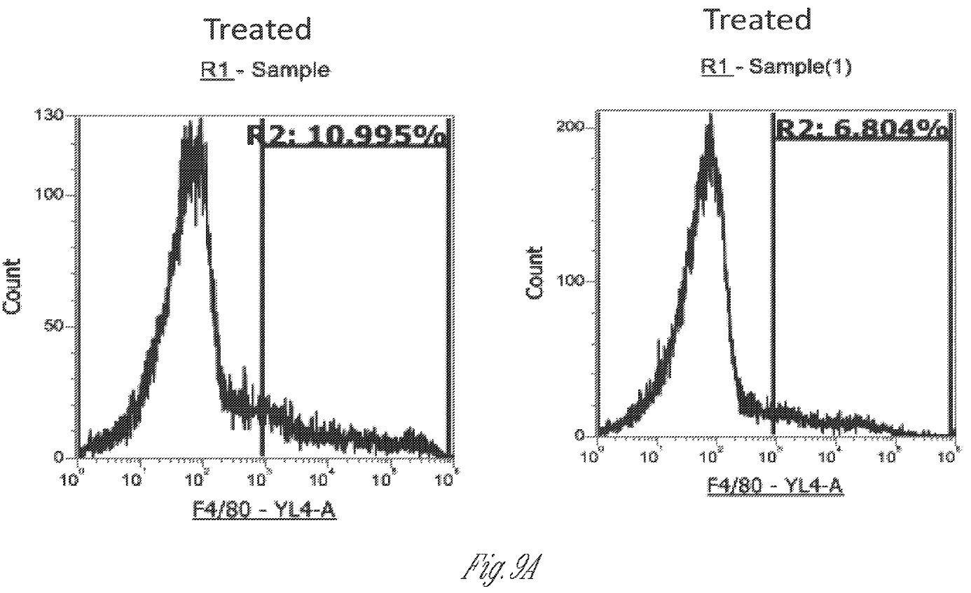

The compound shown in FIG. 1 can be synthesized via the scheme shown in FIG. 4 by reacting the activated dexamethasone synthesized via the scheme shown in FIG. 2 with the compound shown in FIG. 3. Briefly, one equivalent of 6-chloro-1H-benzo[d][1,2,3]triazol-1-yl (2-(pyridin-2- yldisulfaneyl)ethyl) carbonate was reacted with 1 equivalent of dexamethasone (Cayman Chemical Co., Ann Arbor, MI, USA) in dimethylsulfoxide (DMSO) overnight at room temperature. The product was purified by reverse phase high- pressure liquid chromatography (RPHPLC) and verified by liquid chromatography-mass spectrometry (LC-MS).

### Example 2

The compound in FIG. 3 was synthesized via solid phase peptide synthesis (SPPS) using standard SPPS protocols along with N,N,N′,N′- tetramethyl-O-(1H-benzotriazol-1-yl)uronium hexafluorophosphate (HBTU) and the coupling reagent. The product was purified by RPHPLC and verified by LC- MS.

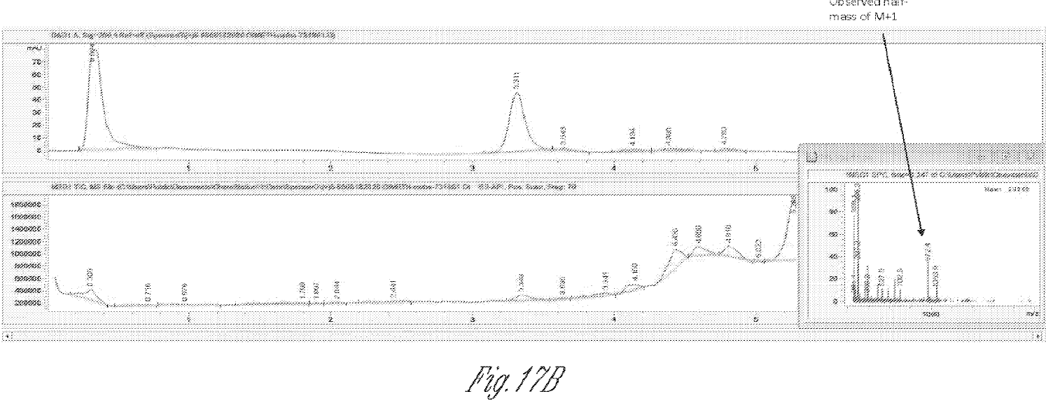

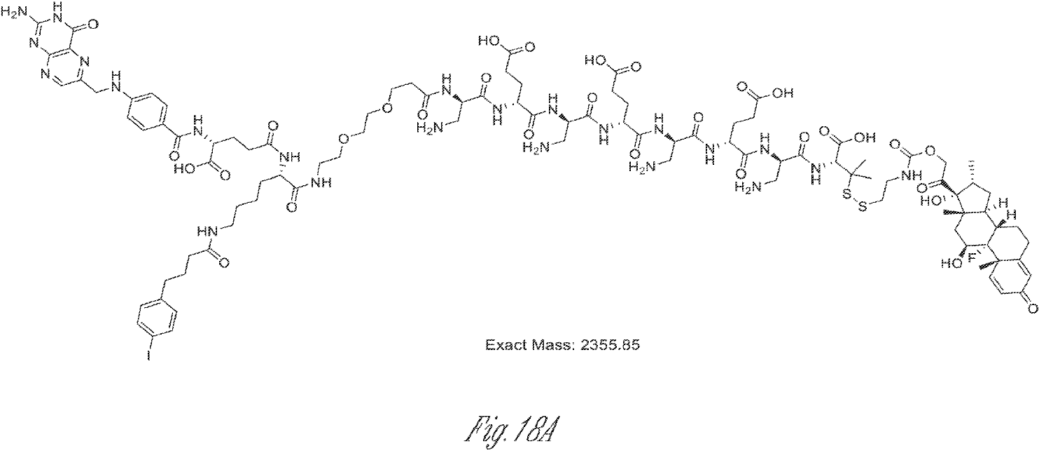

The product of the reaction scheme in FIG. 2 is then reacted with the compound in FIG. 3 to give the compound in FIG. 4. Briefly, one equivalent of the compound in FIG. 3 was dissolved in ammonium acetate buffer, pH 6.5. Then the activated dexamethasone (1 eq) was dissolved in tetrahydrofuran (THF). The THF was added to the buffer containing the compound in FIG. 3 and allowed to stir at room temperature overnight. The reaction was then purified by RPHPLC and verified by LC-MS. The LC-MS trace of the product compound 101 in FIG. 4 is shown in FIG. 16.

### Example 3

### General Solid Phase Peptide Synthesis of Folate-Linker Compounds

As shown in scheme 6, 2-chlorotrityl chloride resin was swelled with anhydrous dichloromethane, then loaded with a solution containing 2eq of Fmoc-S-trityl-L-penicillamine (Fmoc-Pen(trt)-OH aka Fmoc-DimethylCys-OH) and 10eq or either N-methyl morphiline (NMM) or DIPEA. This was allowed to stir under argon in a solid phase peptide synthesis vessel for 2 hrs at room temperature followed by the addition of 10% total volume of methanol for 20 minutes to cap the resin. The resin was then washed with DCM/DMF/DCM, dried and loaded onto an automated solid phase peptide synthesis machine. The remainder of the molecule was synthesized via solid phase peptide synthesis (SPPS) using standard SPPS protocols along with N,N,N′,N′-Tetramethyl-O-(1H-benzotriazol-1-yl)uronium hexafluorophosphate (HBTU) / 1-[Bis(dimethylamino)methylene]-1H-1,2,3-triazolo[4,5-b]pyridinium 3-oxid hexafluorophosphate (HATU) as the coupling reagents. N¹⁰-trifluoroacetylated (N¹⁰-TFA) pteroic acid was coupled last using previously mentioned standard SPPS protocol, followed by N¹⁰-TFA deprotection using 50% ammonia hydroxide in DMF. The product was cleaved from the resin using TFA/TIPS/TCEP-HCl and precipitated in diethyl ether, centrifuged and washed three times with diethyl ether and verified by LC-MS.

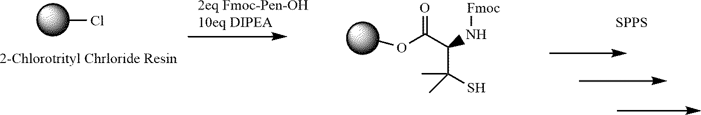

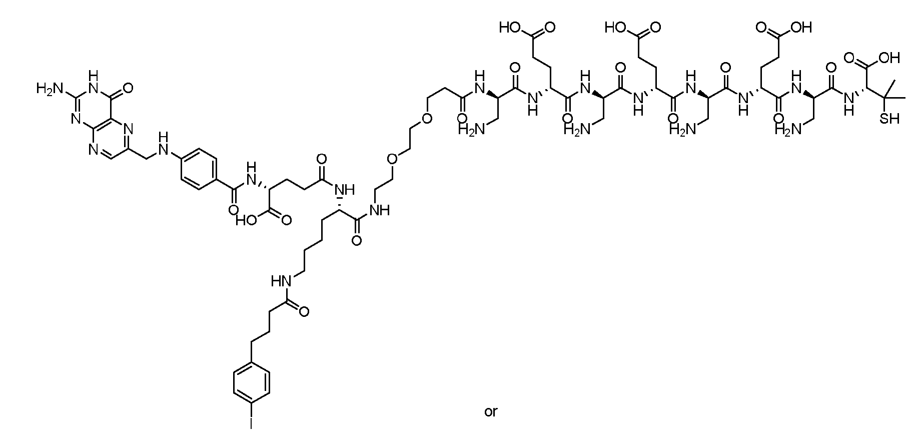

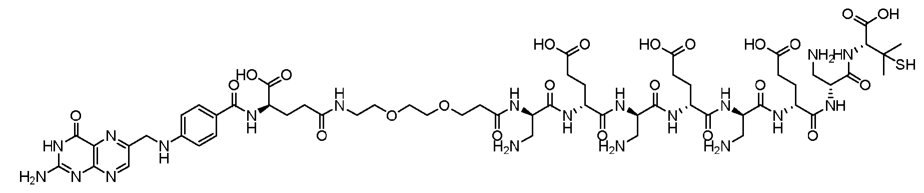

Scheme 6- Synthetic Scheme of Steroid-Carbamate-Pyridyl Disulfide Compounds

### Example 4

General Procedure for Synthesis of Steroid-Carbamate-Pyridyl Disulfide Compounds: 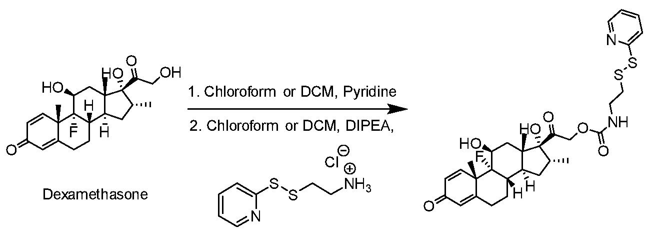 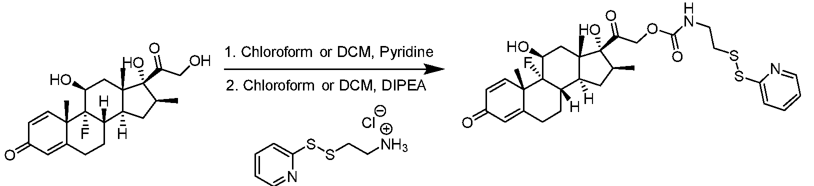 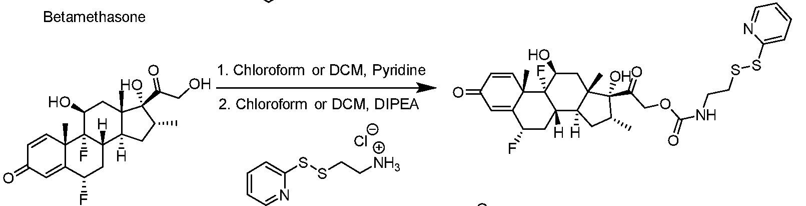 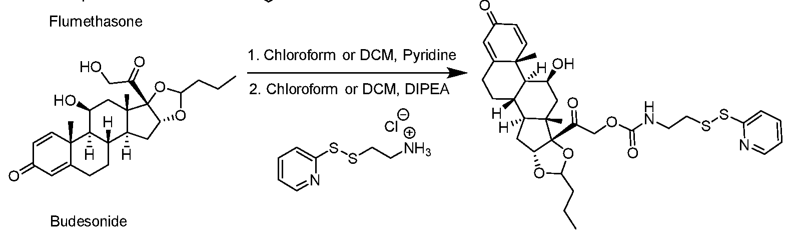 Scheme 7 - Synthetic Scheme of Steroid-Carbamate-Pyridyl Disulfide Compounds

As shown in Scheme 7. Steroid (1 eq, Dexamethasone, Betamethasone, Flumethasone or Budesonide) and p-nitrophenyl chloroformate (1eq) were loaded into a capped vial containing a magnetic stir bar. Next, addition of either chloroform or dichloromethane created a final steroid concentration of 0.1 M. Lastly, ~25eq of pyridine was added to the stirring solution and the reaction was monitored at room temperature by LC-MS. Then, ~1.4 eq of 2-(pyridin-2-yldisulfaneyl)ethan-1-amine acid chloride salt was mixed in a separate container with chloroform or dichloromethane and ~4.3 eq of diisopropylethylamine (DIPEA) and continued to be monitored by LC-MS. Upon completion, the reaction was purified by flash chromatography utilizing either a 0-100% hexane/ethyl acetate or 0-20% dichloromethane/methanol gradient. The products were verified by LC-MS.

### Synthesis of 2-(Pyridin-2-Yldisulfaneyl)Ethan-1-Amine Acid Chloride Salt

25.6 mmols of methoxycarbonyl sulfenyl chloride in 10 mL of acetonitrile was cooled to 0° C., followed by the addition of 25.6 mmols of 2-aminoethanol hydrochloride. Then 23 mmols of 2-mercaptopyridine in 15 mL of methanol was added and the mixture was refluxed for 2 hrs. The vessel was then cooled to 0° C. for one hour and the resulting precipitate was collected by filtration. (2.5660 g yield)

### Synthesis of Dex-Carbamate-Pyridyl Disulfide

392 mg Dexamethasone and 230 mg p-nitrophenyl chloroformate were loaded into a capped vial containing a magnetic stir bar. The solids were dissolved in 10 mL chloroform followed by the addition of 1 mL pyridine, the reaction was run at room temperature and monitored by LC-MS. Then, 320 mg of 2-(pyridin-2-yldisulfaneyl)ethan-1-amine acid chloride salt was mixed in a separate container with 2.5 mL chloroform with 752 uL of diisopropylethylamine (DIPEA) and continued to be monitored by LC-MS. Upon completion, the reaction was purified by flash chromatography utilizing a 0-20% dichloromethane/methanol gradient. The product was verified by LC-MS. (460.0 mg yield)

### Synthesis of Beta-Carbamate-Pyridyl Disulfide

10.0 mg Betamethasone and 5.4 mg p-nitrophenyl chloroformate were loaded into a capped vial containing a magnetic stir bar. Next, addition of 200 uL dichloromethane followed by 40 uL of pyridine was added to the stirring solution and the reaction was monitored at room temperature by LC-MS. Then, 5.7 mg of 2-(pyridin-2-yldisulfaneyl)ethan-1-amine acid chloride salt was mixed in a separate container with 500 uL dichloromethane and 13.3 uL of diisopropylethylamine (DIPEA) and continued to be monitored by LC-MS. Upon completion, the reaction was purified by flash chromatography utilizing a 0-20% dichloromethane/methanol gradient, then a 0-100% hexane/ethyl acetate. The product was verified by LC-MS. (6.2 mg yield)

### Synthesis of Flu-Carbamate-Pyridyl Disulfide

10.6 mg Flumethasone and 5.4 mg p-nitrophenyl chloroformate were loaded into a capped vial containing a magnetic stir bar. 213 uL of dichloromethane was added followed by 42.7 uL of pyridine and the reaction was monitored at room temperature by LC-MS. Then, 5.75 mg of 2-(pyridin-2-yldisulfaneyl)ethan-1-amine acid chloride salt was mixed in a separate container with 500 uL of dichloromethane and 12.5 uL of diisopropylethylamine (DIPEA) and continued to be monitored by LC-MS. Upon completion, the reaction was purified by flash chromatography utilizing 0-20% dichloromethane/methanol gradient. The product was verified by LC-MS. (5.8 mg yield)

### Synthesis of Bud-Carbamate-Pyridyl Disulfide

25 mg of Budesonide and 12.3 mg p-nitrophenyl chloroformate were loaded into a capped vial containing a magnetic stir bar. 500 uL chloroform and 100 uL of pyridine were added to the stirring solution and the reaction was monitored at room temperature by LC-MS. Then, 12.9 mg of 2-(pyridin-2-yldisulfaneyl)ethan-1-amine acid chloride salt was mixed in a separate container with 500 uL chloroform and 30.3 uL of diisopropylethylamine (DIPEA) and was monitored by LC-MS. Upon completion, the reaction was purified by flash chromatography utilizing 0-20% dichloromethane/methanol gradient. The products was verified by LC-MS. (30.6 mg yield)

### Example 5

General Synthesis of Folate-Albuminbinder-PEG2-Hydrolink-DimethylCysteine-Carbamate-Steroid Compounds 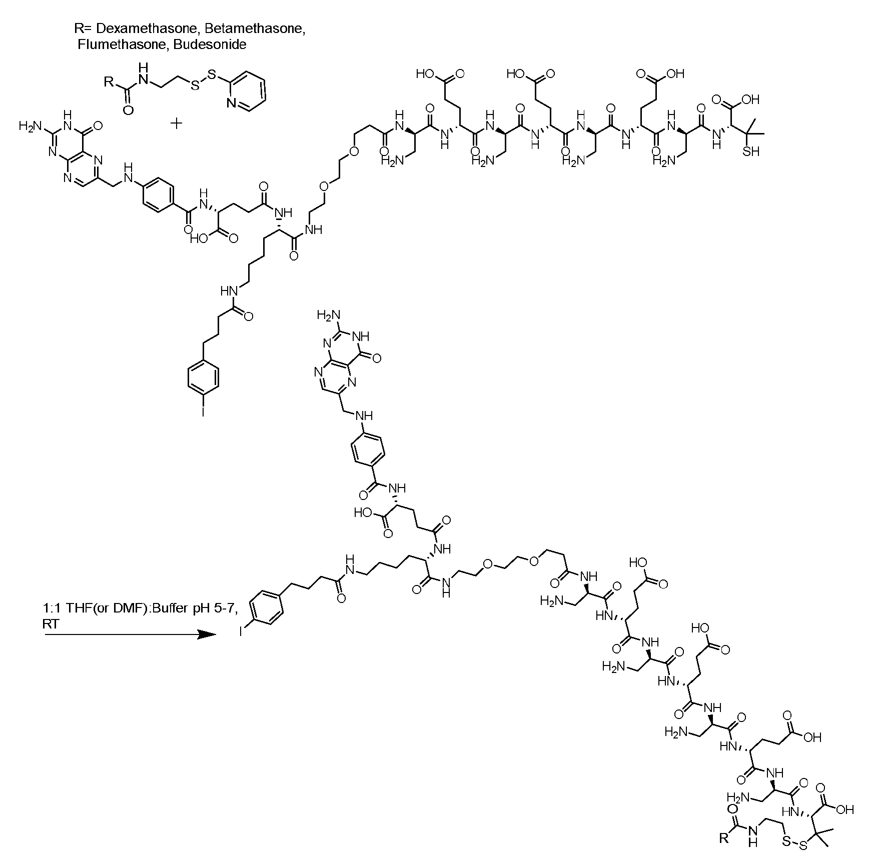 Scheme 8 Final DimethylCys-Maleimide Coupling of Compounds.

### General Synthesis of Folate-Albuminbinder-PEG2-Hydrolink-DimethylCysteine-Carbamate-Steroid Compounds

As shown in scheme 8. 1-2eq of Fol-Alb-PEG2-Hydrolink-DimethylCys was dissolved in 20 mM ammonium acetate buffer (pH 5-7) and stirred vigorously. Then, 1eq of Steroid-Carbamate-Pyridyl Disulfide dissolved in THF or DMF (volume equal to buffer) was added quickly and monitored to completion by LC-MS. The reaction as then purified by RPHPLC utilizing 20 mM ammonium acetate buffer/acetonitrile and verified by LC-MS.

### Compound 107 (Folate-Albuminbinder-PEG2-Hydrolink-DimethylCysteine-Carbamate-Dexamethasone)

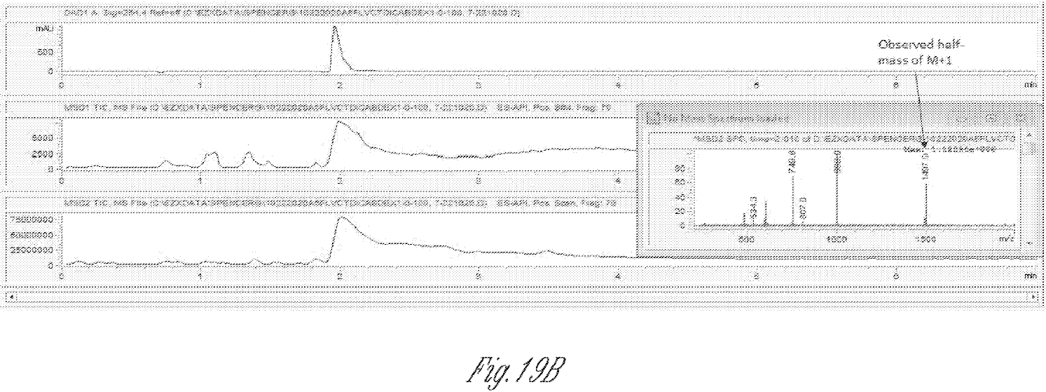

60 mg of Fol-Alb-PEG2-Hydrolink-DimethylCys was dissolved in 1 mL 20 mM ammonium acetate buffer (pH 6) and stirred vigorously. Then, 19.2 mg of Dex-Carbamate-Pyridyl Disulfide dissolved in 1 mL THF was added quickly and monitored to completion by LC-MS. The reaction as then purified by RPHPLC utilizing 20 mM ammonium acetate buffer/acetonitrile and verified by LC-MS. (Compound 107; 15.8 mg yield). LC-MS is shown in FIG. 18.

### Compound 124 (Folate-Albuminbinder-PEG2-Hydrolink-DimethylCysteine-Carbamate-Flumethasone)

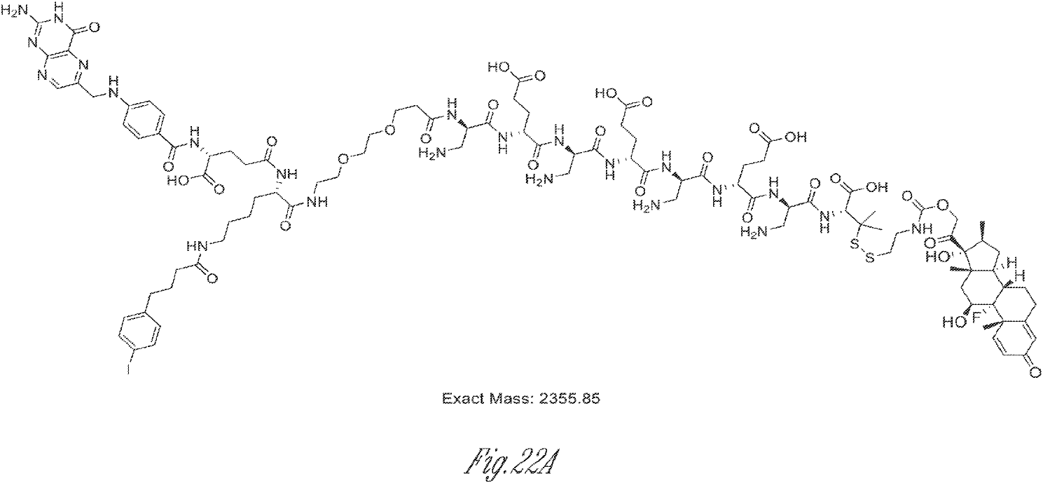

26 mg of Fol-Alb-PEG2-Hydrolink-DimethylCys was dissolved in 250 uL 20 mM ammonium acetate buffer (pH 6.19) and stirred vigorously. Then, 5.8 mg of Flu-Carbamate-Pyridyl Disulfide dissolved in 250 uL THF was added quickly and monitored to completion by LC-MS. The reaction as then purified by RPHPLC utilizing 20 mM ammonium acetate buffer/acetonitrile and verified by LC-MS. (Compound 124; 1 mg yield) LC-MS is shown in FIG. 21.

### Folate-Albuminbinder-PEG2-Hydrolink-DimethylCysteine-Carbamate-Betamethasone (Compound 126)

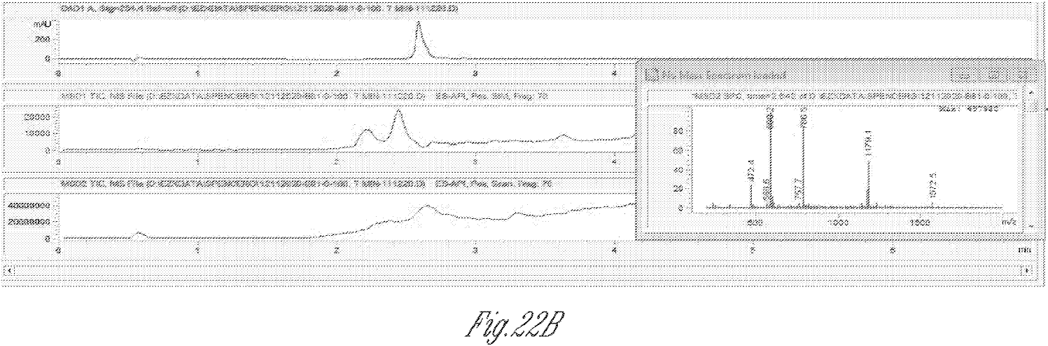

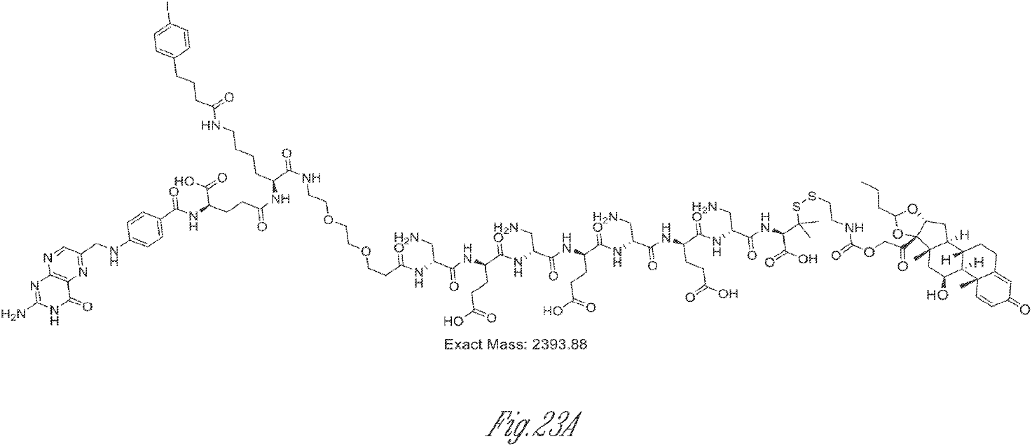

28.7 mg of Fol-Alb-PEG2-Hydrolink-DimethylCys was dissolved in 0.250 mL 20 mM ammonium acetate buffer (pH 6) and stirred vigorously. Then, 6.2 mg of Betamethasone-Carbamate-Pyridyl Disulfide dissolved in 0.250 mL THF was added quickly and monitored to completion by LC-MS. The reaction as then purified by RPHPLC utilizing 20 mM ammonium acetate buffer/acetonitrile and verified by LC-MS. (Compound 126; 2.6 mg yield). LC-MS is shown in FIG. 22.

### Folate-Albuminbinder-PEG2-Hydrolink-DimethylCys-Carbamate-Budesonide (Compound 127)

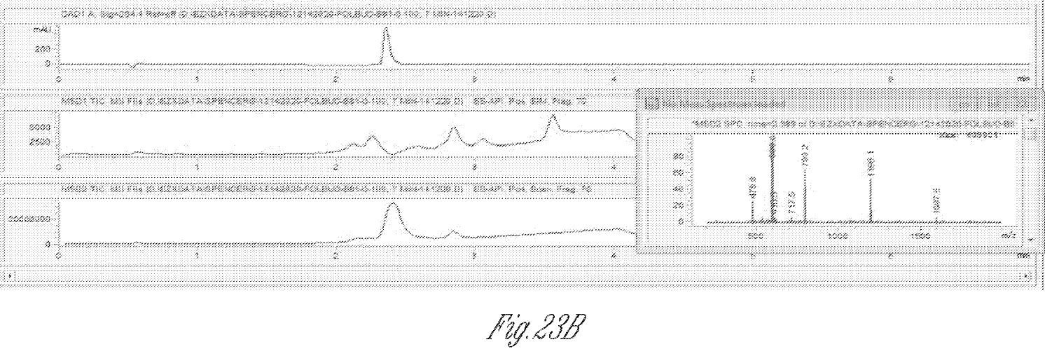

21.8 mg of Fol-Alb-PEG2-Hydrolink-DimethylCys was dissolved in 0.250 mL 20 mM ammonium acetate buffer (pH 6) and stirred vigorously. Then, 5 mg of Betamethasone-Carbamate-Pyridyl Disulfide dissolved in 0.250 mL THF was added quickly and monitored to completion by LC-MS. The reaction as then purified by RPHPLC utilizing 20 mM ammonium acetate buffer/acetonitrile and verified by LC-MS. (Compound 127; 3.0 mg yield). LC-MS is shown in FIG. 23.

Synthesis of Compound 106 (Folate-PEG2-Hydrolink-DimethylCysteine-Carbamate-Dexamethasone) Scheme 9 - Synthetic scheme for Folate-PEG2-Hydrolink-DimethylCysteine-Carbamate-Dexamethasone (Compound 106)

[symbol]

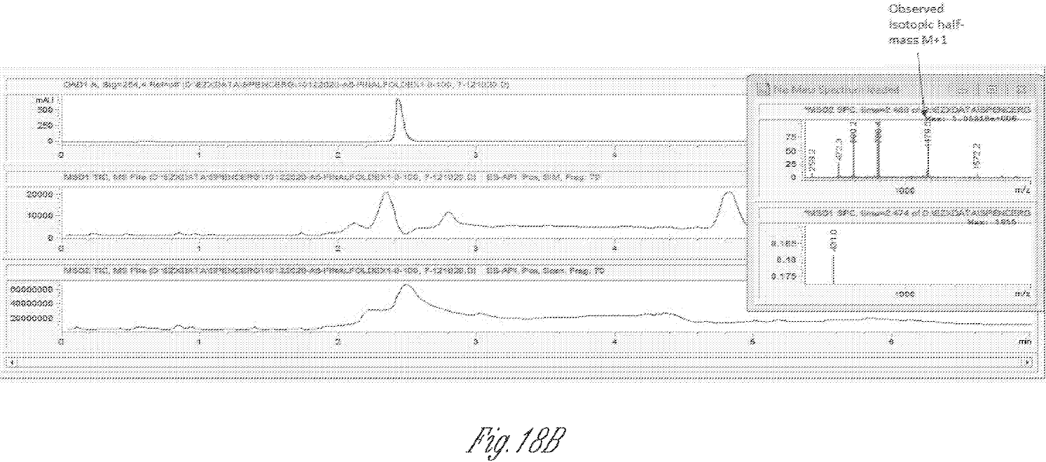

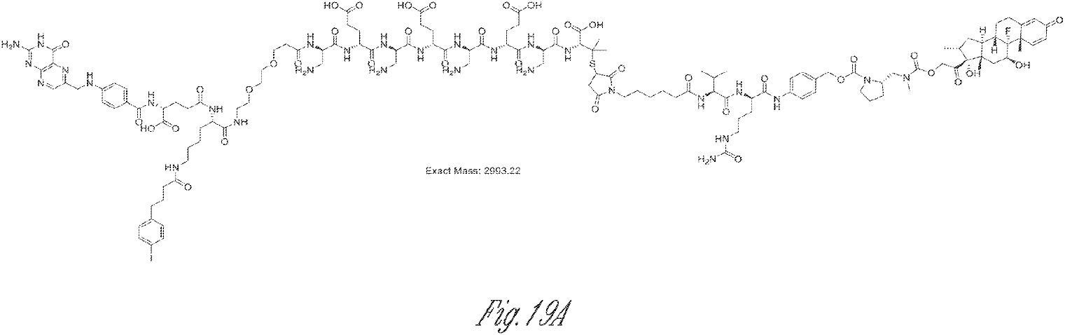

39.2 mg of dexamethasone and 23.0 mg of p-nitrophenyl chloroformate were dissolved in 1 mL of chloroform and 100 uL of pyridine. The reaction was run for 2 hrs, washed with water, dilute HCl, water, dried over magnesium sulfate, filtered, dried de vacuo and triturated with diethyl ether. (10.3 mg yield) The product was dissolved in 150 uL of DMF followed by 7.3 uL DIPEA and 4.7 mg of 2-(pyridin-2-yldisulfaneyl)ethan-1-amine acid chloride salt. The reaction was monitored by LC-MS and upon completion the crude reaction mixture was precipitated with diethyl ether, dried, dissolved in 300 uL DMF and 300 uL of 20 mM ammonium acetate buffer (pH 6) containing 20 mg Fol-mPEG2-Hydrolink-DimethylCys was added quickly and monitored to completion by LC-MS. The reaction as then purified by RPHPLC utilizing 20 mM ammonium acetate buffer/acetonitrile and verified by LC-MS. LC-MS of compound 106 is shown in FIG. 17.

### Example 6

Synthesis of Compound 125 (Folate-Albuminbinder-PEG2-Hydrolink-DimethylCysteine-CathepsinBCleavable-Dicarbamate-Dexamethasone) 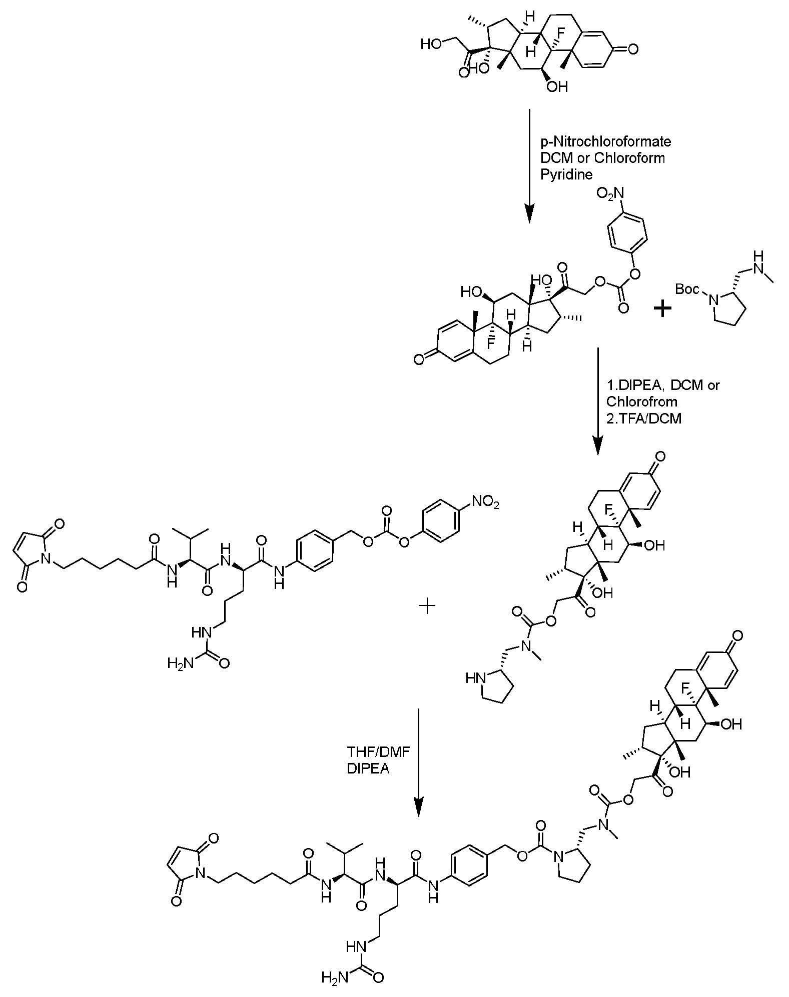 Scheme 10 - Synthetic Scheme for Maleimide-CathepsinBCleavable-Dicarbamate-Dex

### Synthesis of Compound 125 (Folate-Albuminbinder-PEG2-Hydrolink-DimethylCysteine-Maleimide-(CH₂)₅-CathepsinBCleavable-Dicarbamate-Dexamethasone)

As shown in scheme 10. Dexamethasone (1eq) and p-nitrophenyl chloroformate (1eq) was loaded into a capped vial containing a magnetic stir bar. Next, addition of either chloroform or dichloromethane created a final steroid concentration of 0.1 M. Lastly, ~25eq of pyridine was added to the stirring solution and the reaction was monitored at room temperature by LC-MS. Then, ~1eq of tert-butyl (S)-2-((methylamino)methyl)pyrrolidine-1-carboxylate was mixed in a separate container with chloroform or dichloromethane and diisopropylethylamine (DIPEA). Upon completion or reaction monitored by LC-MS, the product was purified by flash chromatography utilizing a 0-20% dichloromethane/methanol gradient to afford tert-butyl (S)-2-((((2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethoxy)carbonyl)(methyl)amino)methyl)pyrrolidine-1-carboxylate.

Next, 12.9 mg of tert-butyl (S)-2-((((2-((8S,9R,10S,11S,13S,14S,16R,17R)-9-fluoro-11,17-dihydroxy-10,13,16-trimethyl-3-oxo-6,7, 8,9,10,11,12,13,14,15,16,17-dodecahydro-3H-cyclopenta[a]phenanthren-17-yl)-2-oxoethoxy)carbonyl)(methyl)amino)methyl)pyrrolidine-1-carboxylate was treated with a 1:1 mixture of trifluoroacetic acid (TFA) and dichloromethane (DCM). Boc protecting group removal was monitored and validated by LC-MS. The TFA/DCM mixture was dried de vacuo and the resulting oil was dissolved in 200 uL of THF plus 600 uL DMF followed by 28.3 uL of DIPEA, to which was added 15 mg of 4-((R)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate supplied by a commercial vendor.

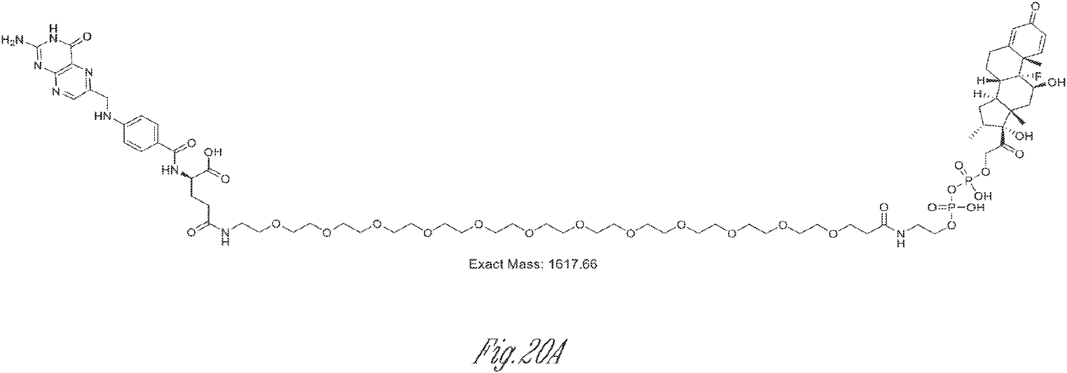

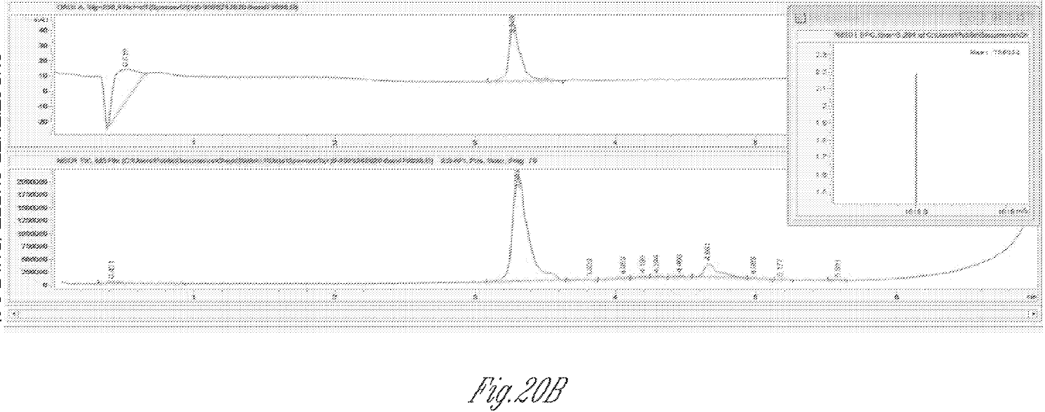

This intermediate was validated by LC-MS, then the reaction mixture was added to 37.9 mg of Fol-Alb-PEG2-Hydrolink-DimethylCys vigorously stirring in 800 uL of pH 5.18 20 mM ammonium acetate buffer, as shown in scheme 10. The product was then purified by RPHPLC 20 mM ammonium acetate buffer pH 7/acetonitrile and verified by LC-MS. (compound 125; 6.74 mg yield). LC-MS is shown in FIG. 19. 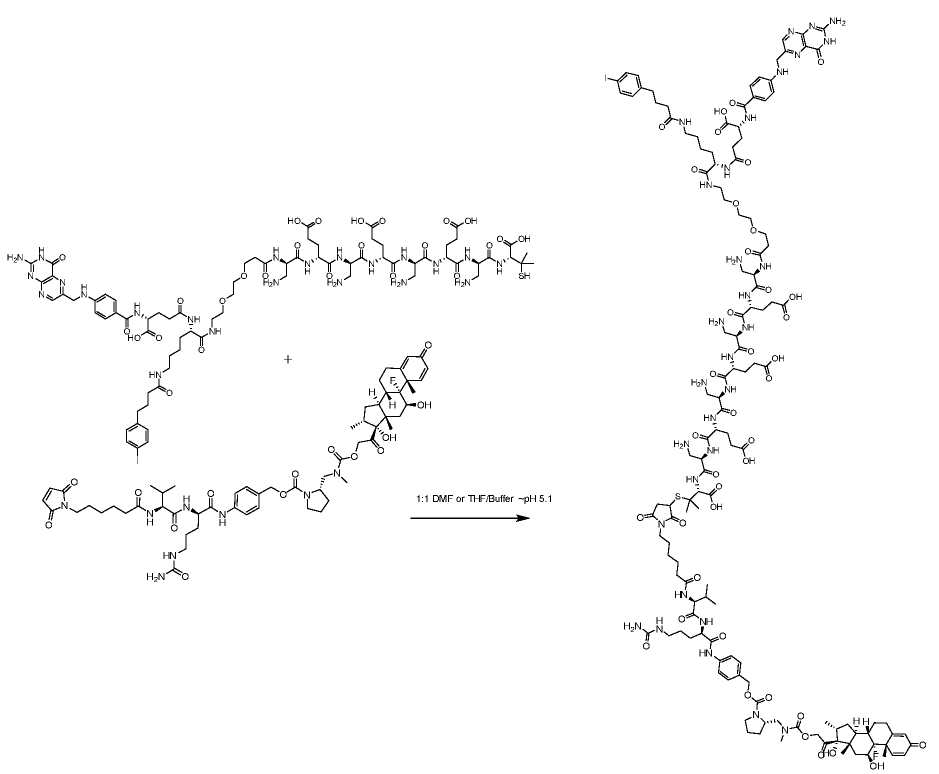 Scheme 11- Synthesis of Compound 125 (Fol-Alb-PEG2-Hydrolink-DimethylCysteine-Maleimide-(CH₂)₅-CathepsinBCleavable-Dicarbamate-Dexamethasone)

### Example 8

Synthesis of Folate-PEG12-Pyrophosphate-Dexamethasone 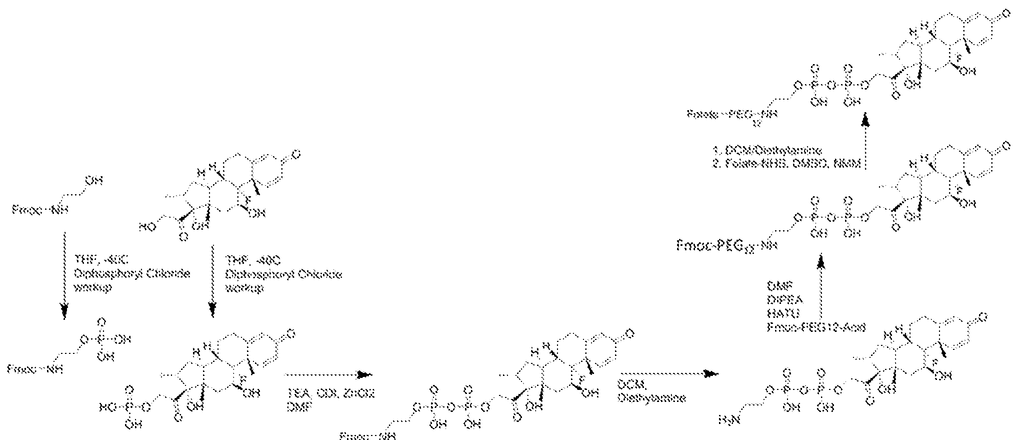 Scheme 12 - Synthetic Scheme of Compound 108 (Folate-PEG12(CH₂)-Pyrophosphate-Dexamethasone)

### Synthesis of Dexamethasone-21 Phosphate

As shown in Scheme 12. 1 g of dexamethasone was added to a 50 mL round bottom flask followed by 5 mL of anhydrous THF. Then, the reaction was cooled to -40° C. with a dry ice/acetonitrile cold bath. 1.06 mL of diphosphoryl chloride was added and the reaction was stirred at -40° C. for 1 hour. The reaction was quenched with water, titrated to ~pH 8 with saturated sodium bicarbonate, then the pH was brought to ~2 with 1 M hydrochloric acid. The resulting precipitate was extracted with ethyl acetate, washed with acidified brine, dried over sodium sulfate then de vacuo and used in the next step without further purification. (1.0231 g yield)

### Synthesis of Fmoc-Phosphate

1 g of (9H-fluoren-9-yl)methyl (2-hydroxyethyl)carbamate was added to a 50 mL round bottom flask followed by 6.8 mL of anhydrous THF. Then, the reaction was cooled to -40° C. with a dry ice/acetonitrile cold bath. 1.2 mL of diphosphoryl chloride was added and the reaction was stirred at -40° C. for 1 hour. The reaction was quenched with water, titrated to ~pH 8 with saturated sodium bicarbonate, then the pH was brought to ~2 with 1 M hydrochloric acid. The resulting precipitate was extracted with ethyl acetate, washed with acidified brine, dried over sodium sulfate then de vacuo and used in the next step without further purification. (1.2870 g yield)

### Synthesis of Fmoc-Pyro-Dex

50 mg of Fmoc-Phosphate was dissolved in 0.4 mL of DMF, followed by the addition of 0.02 mL Trimethylamine and 56.7 mg of carbonyl diimidizole (CDI) for approximately 30 minutes. Then 63.3 mg of dexamethasone-21 phosphate was added, followed by 150 mg of anhydrous zinc (II) chloride. The reaction was monitored by LC-MS and upon completion, acetonitrile and 20 mM ammonium acetate buffer pH 7 were added and was lyophilized. This resulting residue was then dissolved in DMSO and purified by RPHPLC. (27.06 mg yield)

### Synthesis of Fmoc-PEG12-Pyro-Dex

30.5 mg of Fmoc-Pyro-Dex was first dissolved in 10 mL of 6% diethylamine (DEA) in DCM, Fmoc deprotection was monitored by LC-MS. The DEA/DCM mixture was then removed under vacuum. A solution of 31.3 mg Fmoc-PEG12-acid was activeated with 14.1 mg HATU, 14.1 mg Cl-HOBt, 900 uL DMF and 40.9 uL NMM. This mixture was then added to the Fmoc deprotected Pyro-Dex residue and upon reaction completion monitored by LC-MS, purified by RPHPLC 20 mM ammonium acetate buffer pH 7/acetonitrile and verified by LC-MS.

### Synthesis of Folate-NHS

100 mg of folic acid was dissolved in 10 mL of DMSO followed by the addition of 31.3 mg NHS and 46.7 mg N,N′-Dicyclohexylcarbodiimide. The mixture was allowed to stir overnight at room temperature. The product was then precipitated in ethyl acetate, filtered by vacuum filtration and washed three times with diethyl ether. The resulting solid was used without further purification. (103.4 mg yield)

### Synthesis of Compound 108 (Folate-PEG12(CH₂)-Pyrophosphate-Dexamethasone)

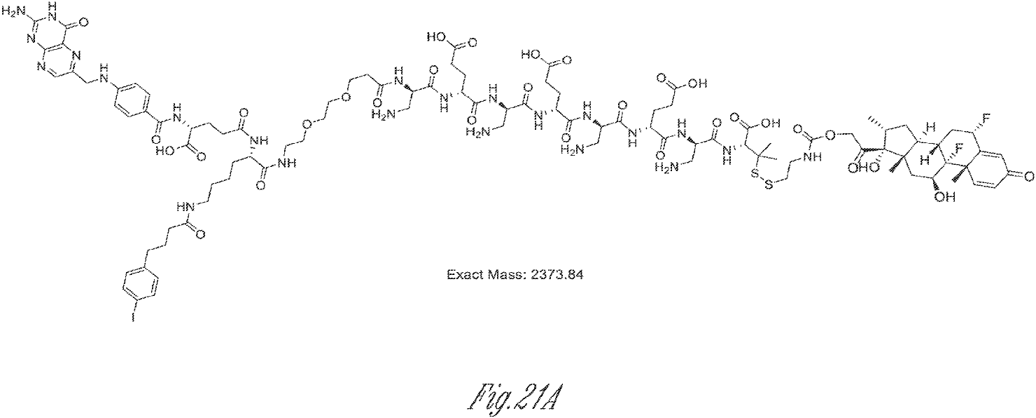

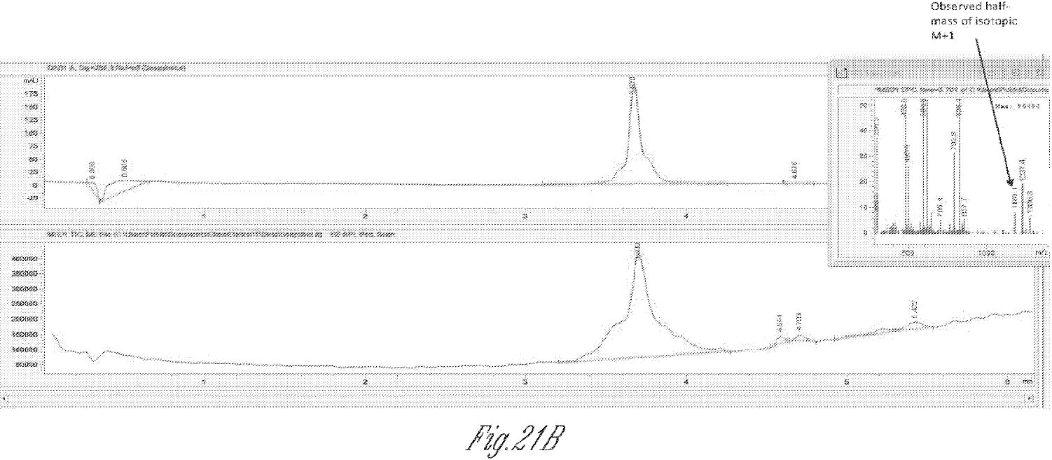

Fmoc-PEG12-Pyro-Dex was then deprotected with DEA/DCM, then dried under vacuum and 2.2 mg of the resulting deprotected compound was mixed with 2 mg of Folate-NHS in 250 uL of DMSO and 1uL of DIPEA. The reaction was monitored by LC-MS and upon completion was purified by RPHPLC and verified by LC-MS. (Compound 108; 1.93 mg yield). LC-MS is shown in FIG. 20.

### Example 9

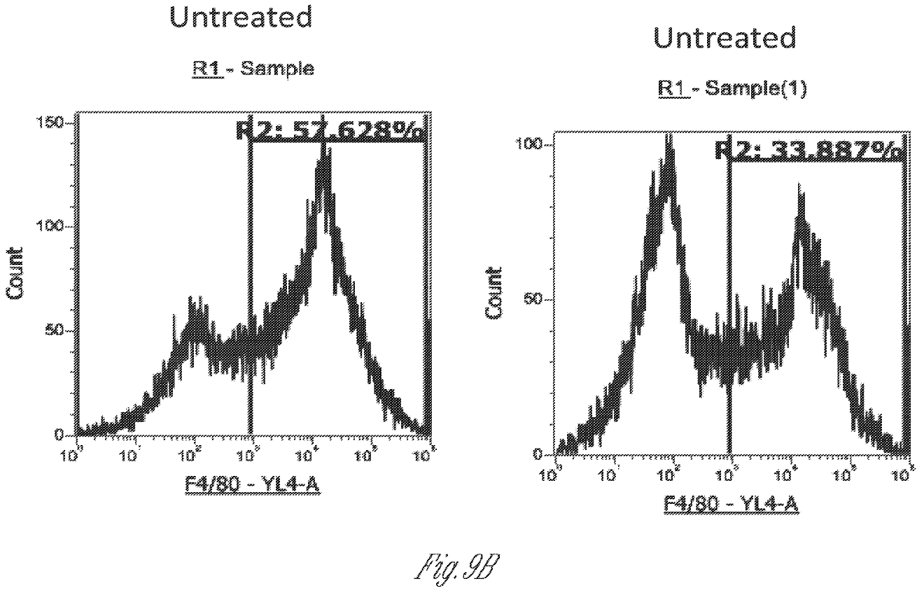

FIG. 6 is an example of compound 101 in flow cytometry of macrophage gated on F4/80 to show CD 206 expression (a marker for M2, anti-inflammatory macrophages). Briefly, 1.5 mL of 3% thioglycolate were injected into the intraperitoneal cavity of 12-week-old ND4 mice. Three days later the macrophages were isolated by intraperitoneal lavage and plated in a 12-well dish and incubated at 37° C./5% CO2. After 24 hours, the media was replaced with media containing 20 nanomolar the compound in FIGS. 1 or 20 nanomolar of the compound in FIG. 1 plus 100-fold excess folate-glucosamine (to show receptor-specific binding) or untreated control or 10 nanomolar free dexamethasone (to show the effect of the free drug) and allowed to incubate at 37° C./5% CO2 for the allotted time. Then 3 days later the cells were detached and incubated with the corresponding fluorescently labeled antibodies for 1 hour at 4° C. in 1% fetal bovine serum (FBS) in phosphate- buffered saline (PBS). The samples were then washed 3 times with ice-cold 1% FBS in PBS before being analyzed by flow cytometry.

Increased expression of CD 206 in the targeted group over the competition group showed receptor-specific uptake of the drug in macrophages.

### Example 10

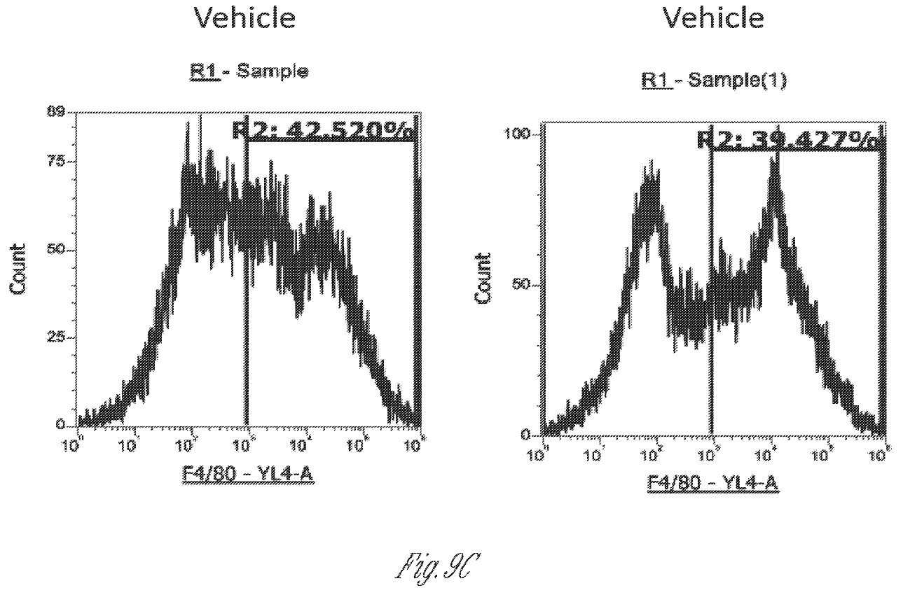

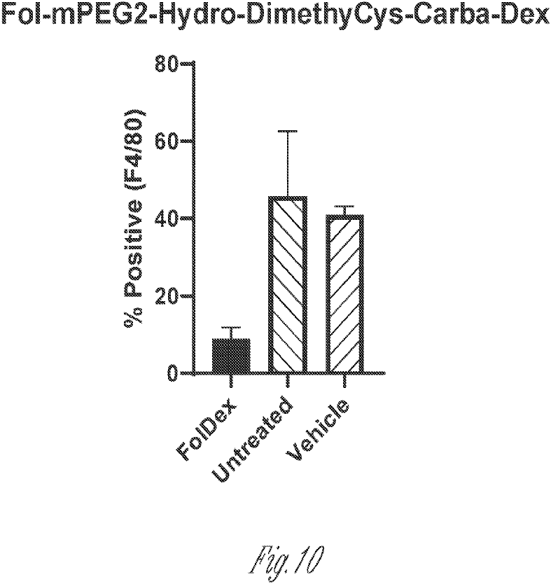

FIG. 7 is another example of Compound 101 in flow cytometry of macrophage gated on F4/80 to show CD 206 expression with controls.

### Example 11

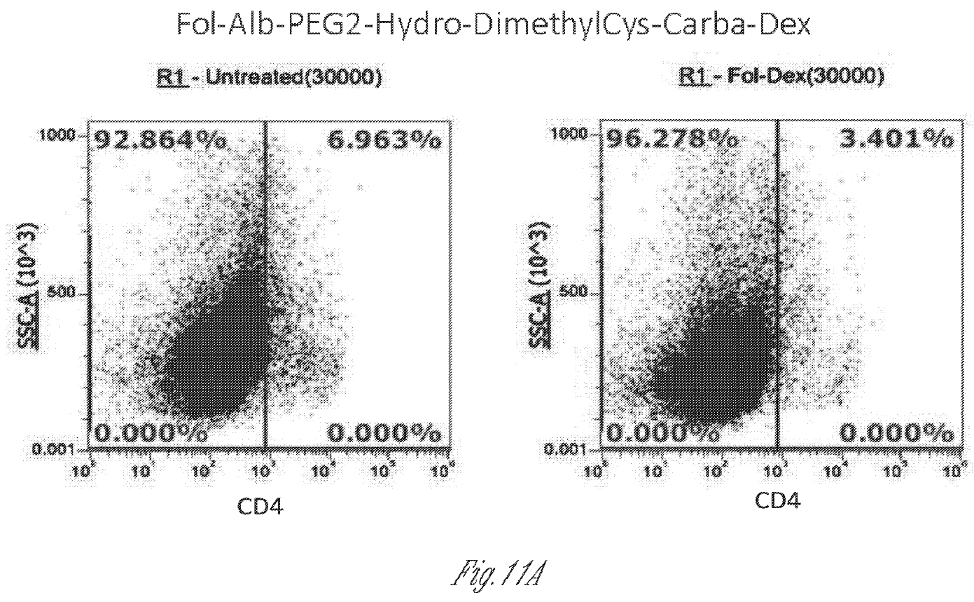

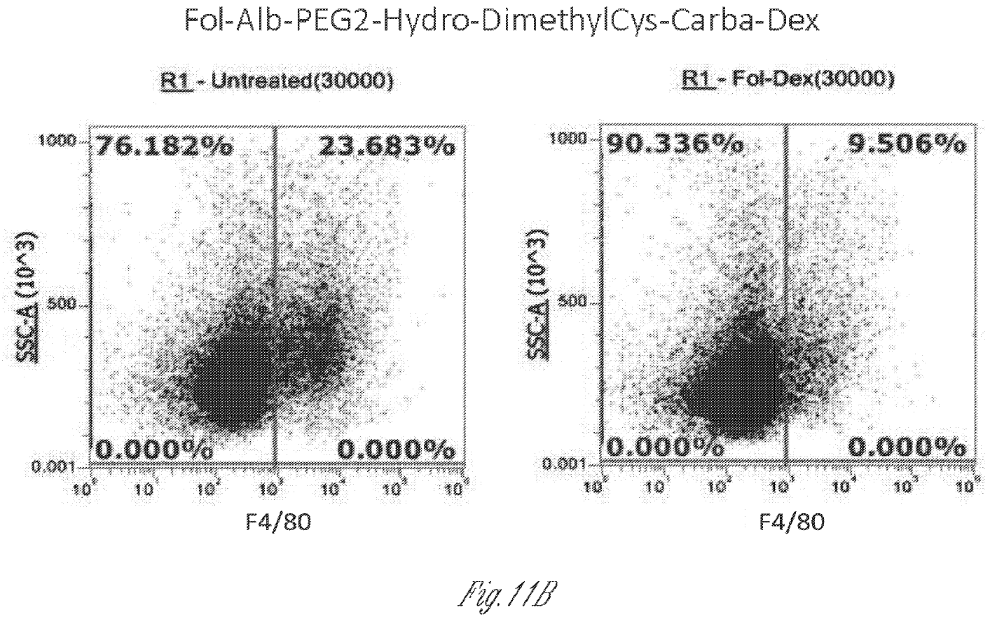

FIG. 8 is an example of Compound 101 in flow cytometry of macrophage to show CD 86 expression with controls. CD 86, a marker for M1, pro-inflammatory macrophages, has no observable differences with various group of compound treatment, competition or free dexamethasone treatment for 24 hours, indicating that the steroid has no effect on M1 CD 86 expression. This indicates that dexamethasone is not having an effect of pro- inflammatory macrophages.

### Example 12

### Mechanistic Studies Using Peritonitis

Induced peritonitis with 1.5 mL 3% thioglycollate injected I.P. into either BALB/C or Swiss Webster mice maintained on a folate free diet for at least three weeks and treated with 10 nmol Compound 107 injected I.V. 48 hrs after induction. After an additional 48 hrs the mice were euthanized, and peritoneal lavage was performed using 2% fetal bovine serum (FBS) in phosphate buffered saline (PBS). Cells were passed through a 70 µm cell filter to remove debris then counted using a hemocytometer. Approximately 1 million cells pre-incubated in anti-CD16/32 (to block endogenous Fc receptor binding) for one hour on ice in 100 µL of 2% FBS in PBS. Then the cells incubated with at least one of the following fluorescently labeled antibodies for cell surface markers: PE-F4/80 or PE/Cy7-F4/80 (mouse macrophage), FITC-CD4 (T helper cell), eFluor780-CD8 (cytotoxic T cell), and PerCP/Cy5.5-Ly6G (neutrophil) according to the manufacturers recommended procedure on ice for 1 hour. Cells were washed twice with 1 mL of ice cold 2% FBS in PBS then suspended in 2% FBS in PBS. Controls included unstained cells as the negative control as well as the use of 1 drop of compensation beads per dye-antibody (1 µL) for a positive control incubated for 1 hour on ice and washed twice with 1 mL 2% FBS in PBS. All samples were centrifuged at 400xg for 10 min between washes, transfers and after incubation to remove supernatant.

Samples and appropriate controls were analyzed on an Attune NxT Acoustic Focusing Cytometer collecting between 10,000 and 30,000 events per sample utilizing laser lines BL1-A, BL2-A, BL3-A, and YL4-A for FITC-CD4, PE-F4/80, PerCP/Cy5.5-Ly6G and eFluor780-CD8 or PE/Cy7-F4/80, respectively. Flow cytometry data analysis involved first observing the cells run in the experiment using forward and side-scattering light (FSC and SSC, respectively). All observed cells were gated (R1) and analyzed for relative abundance of F4/80 (mouse macrophage), CD4 (T helper cell), CD8 (cytotoxic T cell), and Ly6G (neutrophil) cells within the population. Values from quadrant gating based on negative controls were then represented in bar graph form.

### Treatment With Compound 106 (Folate-PEG2-Hydrolink-DimethylCysteine-Carbamate-Dexamethasone)

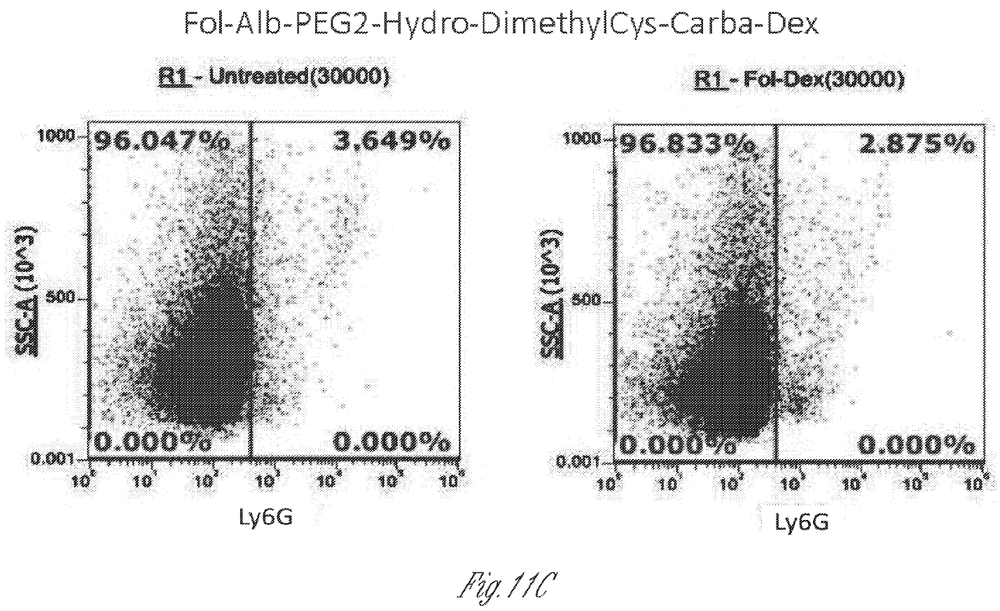

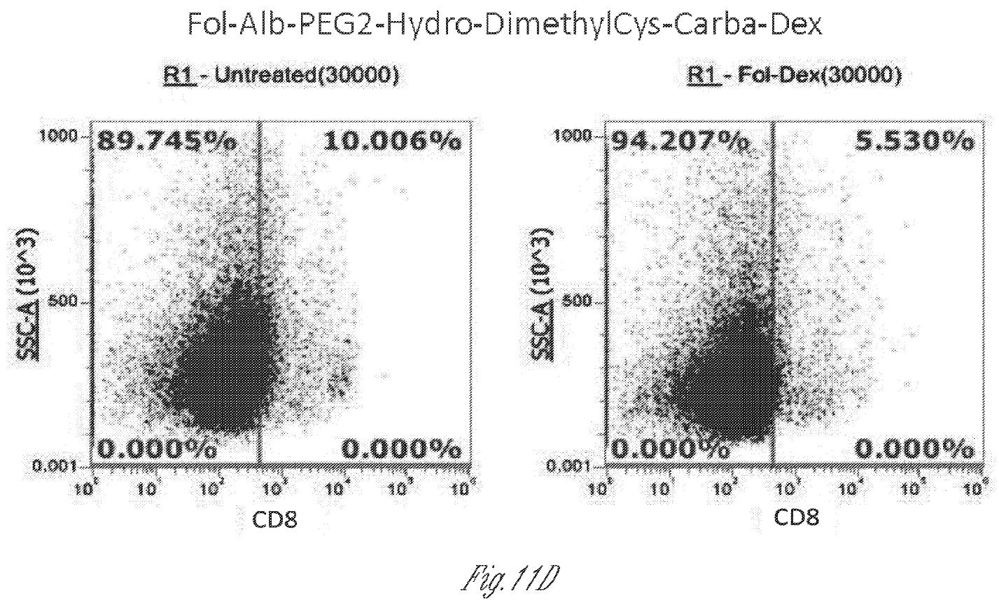

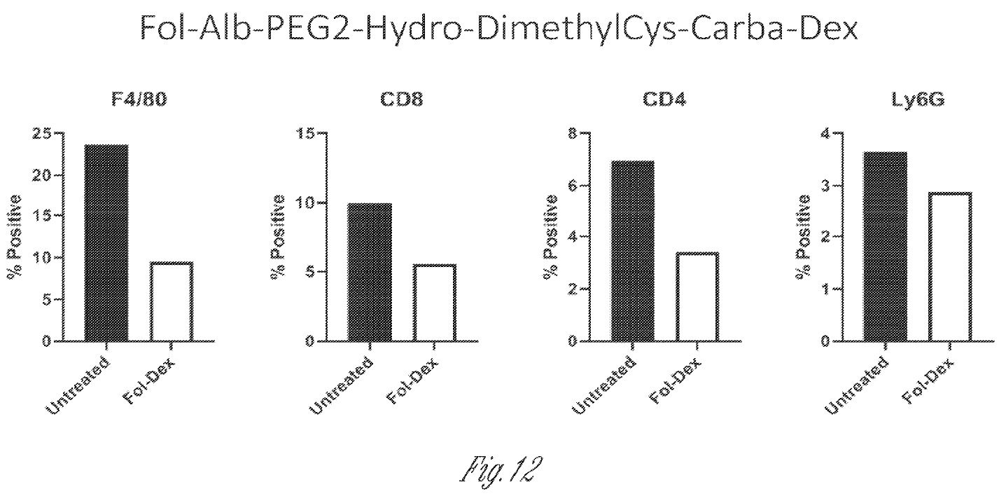

FIG. 9 shows F4/80 cytometry results for one set of samples in the top row and a second set of samples in the bottom row. On the left of each row is a graph with results for cells treated with compound 106, in the middle of each row is a graph with results for untreated cells, and on the right side of each row is a graph with results for vehicle treated cells. FIG. 10 is a bar graph summarizing the results of FIG. 9. As shown by FIG. 9 and FIG. 10, the percentage of F4/80 macrophages is lower in the treated group vs the untreated or vehicle. This shows that there is less inflammation, since macrophages are one of the main mediator of inflammation and there are less of them present in the peritoneal cavity after treatment with compound 106.

### Treatment With Compound 107 (Folate-AlbuminBinder-PEG2-Hydrolink-DimethylCysteine-Carbamate-Dexamethasone)

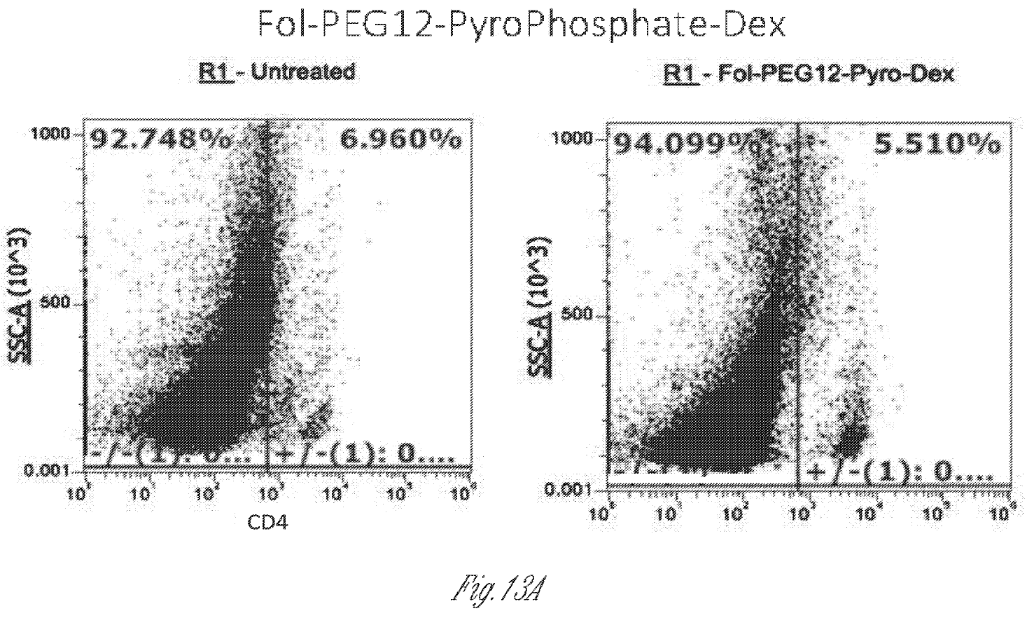

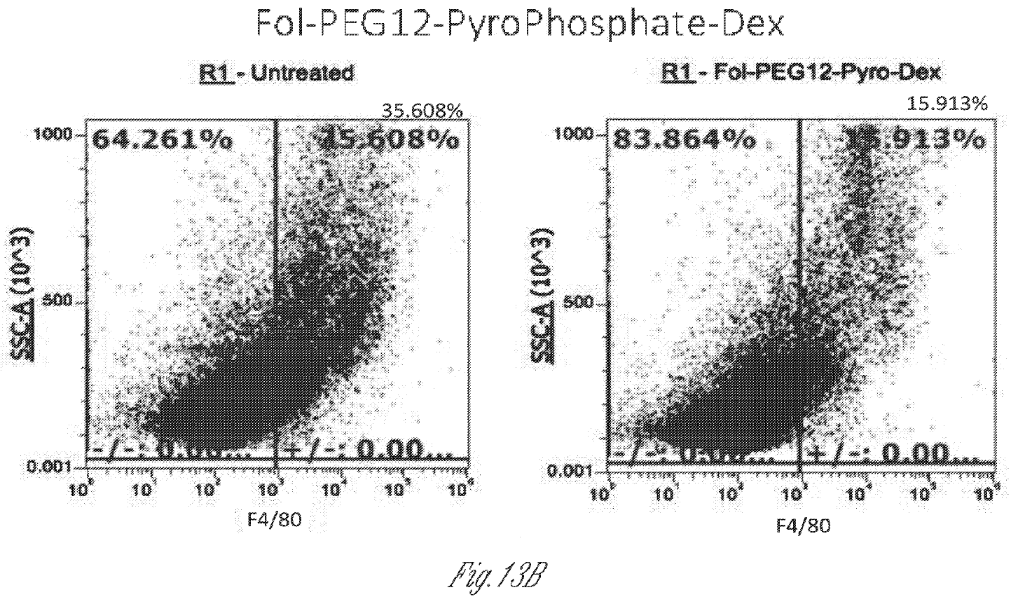

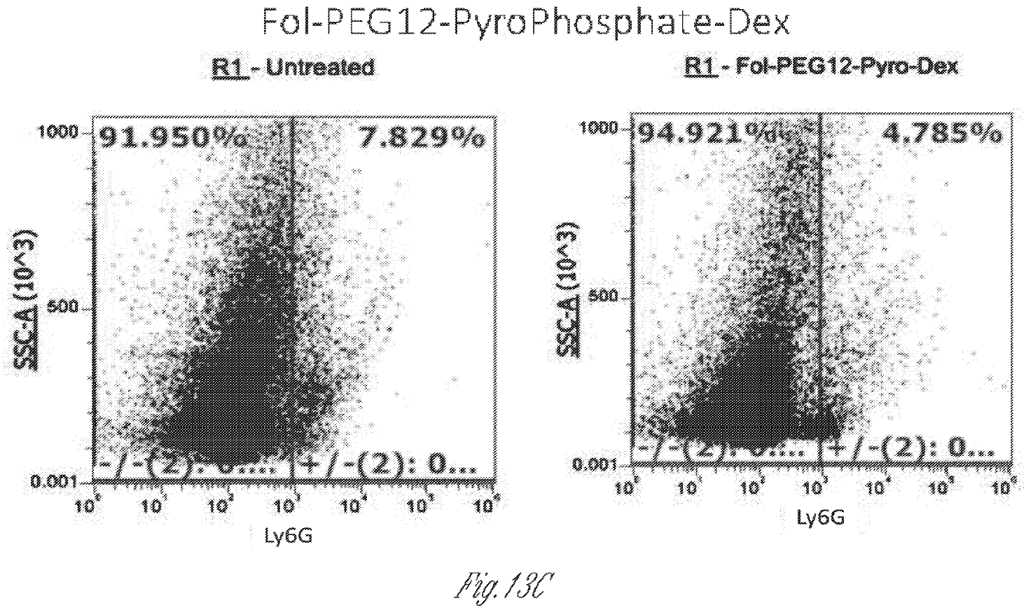

FIG. 11 shows cytometry results for cells which are untreated or treated with compound 107 for four different immune cell types, and FIG. 12 is a bar graph summarizing the results of FIG. 11. As shown in FIG. 11 and FIG. 12, the percentage of PE-F4/80 or (mouse macrophage), FITC-CD4 (T helper cell), eFluor780-CD8 (cytotoxic T cell), and PerCP/Cy5.5-Ly6G (neutrophil) is lower in the treated group vs the untreated or vehicle. The immune cells highlighted above represent a major portion of cells involved in inflammation and there are fewer of them present in the peritoneal cavity after compound 107 treatment.

### Treatment With Compound 108 (Folate-PEG12(CH₂)-Pyrophosphate-Dexamethasone)

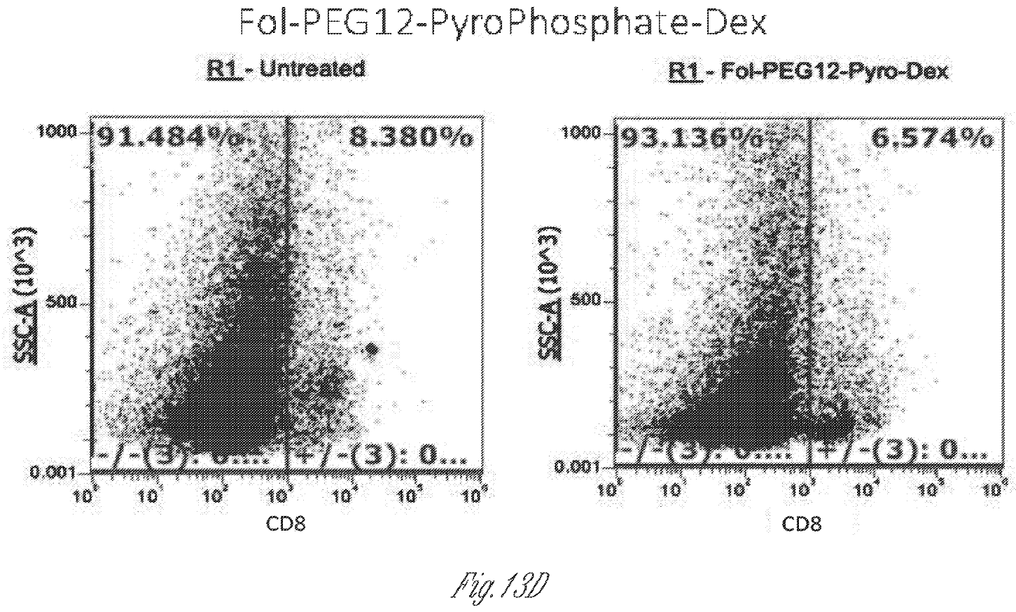

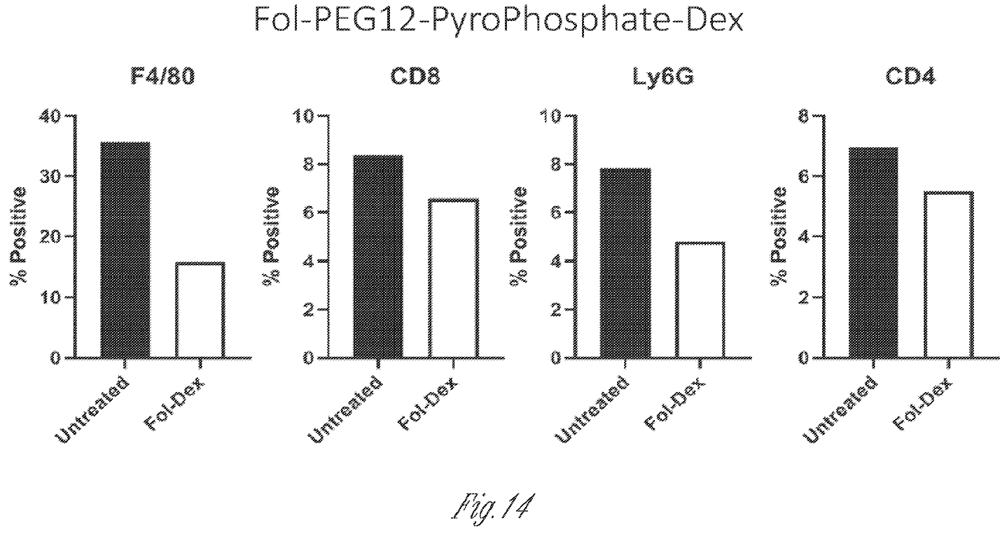

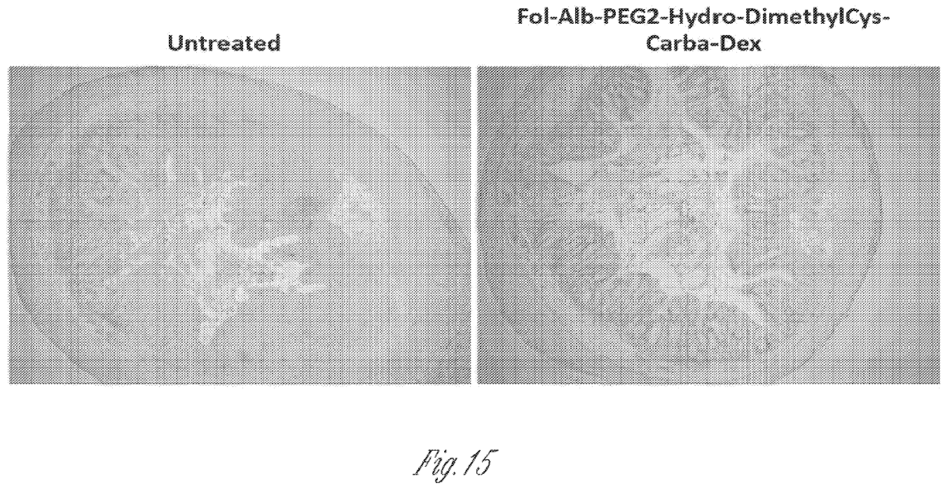

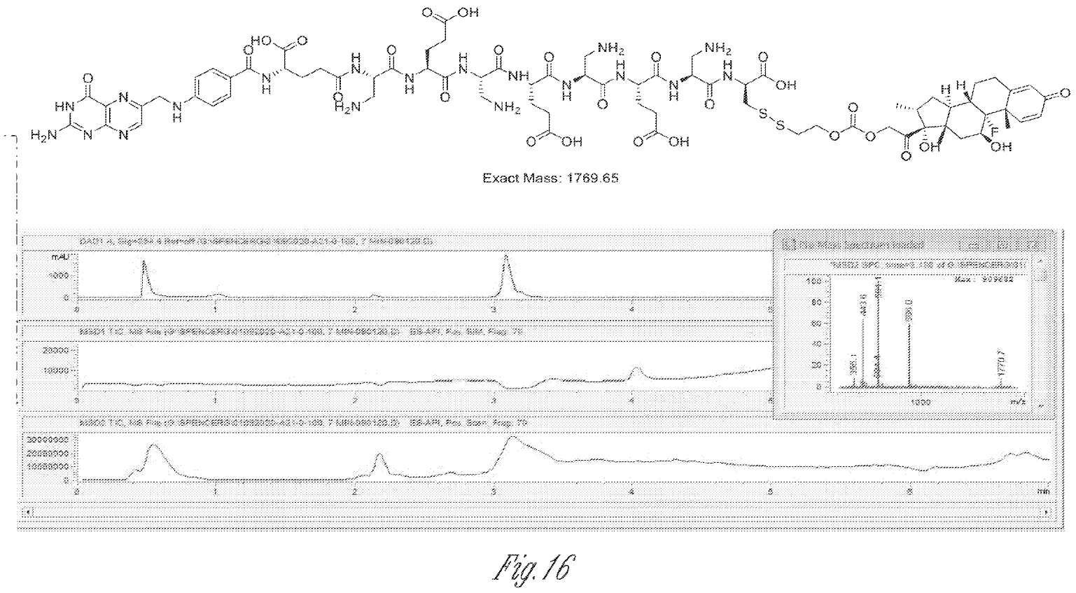

FIG. 13 shows cytometry results for cells which are untreated or treated with compound 108 for four different immune cell types, and FIG. 14 is a bar graph summarizing the results of FIG. 13. As seen above and in the corresponding bar graph below the percentage of PE-F4/80 or (mouse macrophage), FITC-CD4 (T helper cell), eFluor780-CD8 (cytotoxic T cell), and PerCP/Cy5.5-Ly6G (neutrophil) is lower in the treated group vs the untreated or vehicle. The immune cells highlighted above represent a major portion of cells involved in inflammation and there are fewer of them present in the peritoneal cavity after Compound 108 treatment.

### Example 13

### Ulcerative Colitis

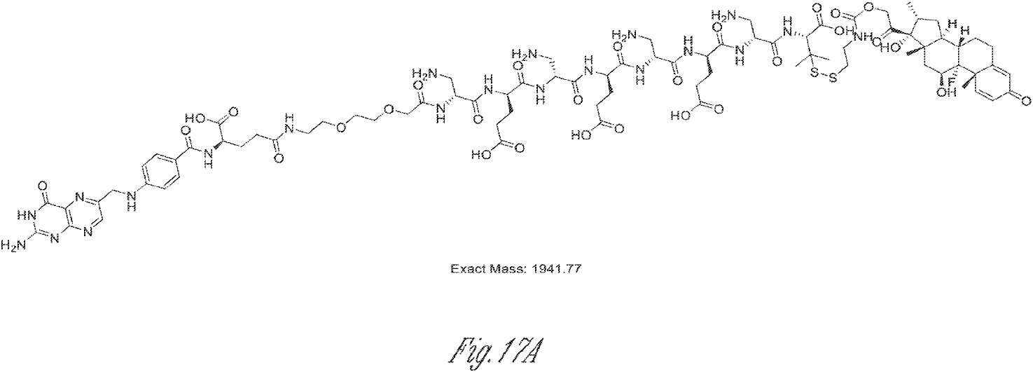

8-12 week old C57BL/6 mice were maintained on a folate free diet for at least three week dextran sodium sulfate (DSS) was added to their drinking water at a final concentration of 2.5% for 6 days. On days 3 and 4 the mice were give a single i.v. injection consisting of 10nmols of Compound 107 in PBS containing 2% DMSO. On day 6 the mice were euthanized by CO2 affixation followed by cervical dislocation. Then the colons were excised and prepared for histology as described previously (J Vis Exp. 2012; (60): 3678.). Briefly, the colons were cut out and the fecal contents were gently removed and washed in PBS. The colons were fixed in 10% buffered formalin for 24 hrs followed by transfer to 70% ethanol. These samples were submitted to the Purdue Veterinary Pathology lab where they were embedded in paraffin, sectioned, and stained with hematoxylin and eosin. The results are shown in FIG. 15, with an image of the untreated colon on the left and an image of the treated colon on the right. Note that the treated group has more crypt architecture, fewer infiltrating immune cells and has a higher number of goblet cells, indicating a healthier colon.

Those skilled in the art will recognize that numerous modifications can be made to the specific implementations described above. The implementations should not be limited to the particular limitations described. Other implementations may be possible.

1. A compound of the formula (I): 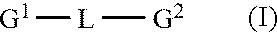
wherein:
G¹ is a folate radical, an antifolate radical, or a folate analog radical;
L is a linker; and
G² is a radical of a steroid;
or a pharmaceutically acceptable salt thereof.

2. The compound of claim 1, wherein the folate radical has the formula: 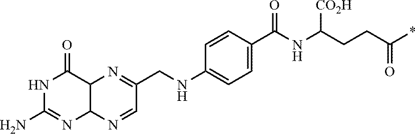
wherein the asterisk denotes the point of attachment of the carbonyl carbon to the linker L.

3. The compound of claim 1, where G¹ is a pteroyl-amino acid radical where the amino acid is selected from the group consisting of aspartic acid, lysine, tyrosine, cysteine, threonine, serine, histidine, arginine, and an unnatural amino acid with a derivatizable moiety in the side chain and/or wherein G¹ is an antifolate radical or a folate analog radical comprising an amino acid selected from the group consisting of aspartic acid, lysine, tyrosine, cysteine, threonine, serine, histidine, and arginine.

4-5. (canceled)

6. The compound of claim 1, wherein G² is a radical of a steroid selected from the group consisting of a radical of dexamethasone, a radical of prednisone, a radical of prednisolone, a radical of methylprednisolone, a radical of budesonide, a radical of triamcinolone; and a radical of betamethasone; and/or wherein G² is selected from:betamethasone, cortisone, cortivazol, difluprednate, hydrocortisone, prednisolone, methylprednisolone, prednisone, dexamethasone, hydrocortisone-17-valerate, budesonide, flumethazone, fluticasone proprionate, fluorocortisone, fludrocortisone, paramethasone, eplerenone, and an ester of any of the foregoing.

7-13. (canceled)

14. The compound of claim 1, wherein the linker is releasable or wherein the linker is non-releasable, and/or
wherein the linker comprises one or more of an amino acid, an alkyl chain, a polyethylene glycol (PEG) monomer, a PEG oligomer, a PEG polymer, or a combination of any of the foregoing, and/or
wherein the linker increases the water solubility of the compound, and/or
wherein the linker comprises an oligomer of peptidoglycans, glycans, anions, or a combination of any of the foregoing, and/or
wherein the linker comprises at least one 2,3-diaminopropionic acid group, at least one glutamic acid group, and at least one cysteine group, and/or
wherein the linker is selected from:
a linker comprising a repeating unit of the formula: 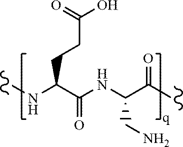
wherein q is an integer from 1 to 10; a linker comprising the formula: 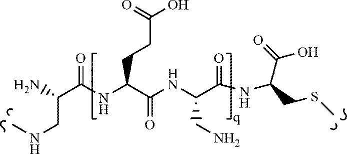
wherein q is an integer from 1 to 10; a linker comprising the formula: 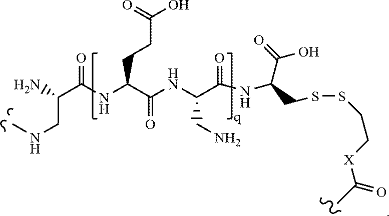
wherein X can be O, NH, NR, or S, and q is an integer from 1 to 10; and a linker comprising the formula: [image]
, and/or wherein the linker is a bivalent linker or wherein the linker is polyvalent and has multiple attachment points for one or more additional chemical groups, and/or
wherein the linker is polyvalent and has multiple attachment points for one or more additional chemical groups and the additional chemical groups comprise one or more additional G¹ groups, and/or
wherein the linker is polyvalent and has multiple attachment points for one or more additional chemical groups and the additional chemical groups comprise one or more binding ligands that are not G¹ groups, and/or
wherein the linker comprises a PEG oligomer with 2-16 PEG units, for example wherein the linker comprises a PEG oligomer with 12 PEG units, and/or
wherein the linker comprises an albumin ligand, for example wherein the albumin ligand comprises 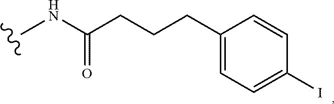
and/or
wherein the linker comprises a dimethylcysteine group, for example wherein the dimethylcysteine group is linked to a succinimide to form: 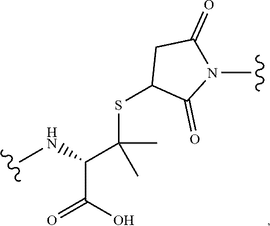
and/or
wherein the linker comprises a phosphate or pyrophosphate group, and/or
wherein the linker comprises a cathepsin B cleavable group, for example wherein the cathepsin B cleavable group is Valine-Citrulline, and/or
wherein the linker comprises a carbamate moiety; or wherein the linker comprises a β-glucuronide; or wherein the linker comprises an ester, phosphate, oxime, acetal, pyrophosphate, polyphosphate, disulfide, sulfate, hydrazide, imine, carbonate, carbamate or enzyme-cleavable amino acid sequence, and/or
wherein the linker comprises a self-immolative moiety.

15-39. (canceled)

40. The compound of claim 1, wherein the linker comprises a self-immolative moiety selected from:
a self-immolative disulfide and or sterically protected disulfide bond;
a self-immolative cathepsin-cleavable amino acid sequence;
a self-immolative furin-cleavable amino acid sequence;
a self-immolative β-glucuronidase-cleavable moiety;
a self-immolative phosphatase-cleavable moiety; and a self-immolative sulfatase-cleavable moiety.

41-46. (canceled)

47. The compound of claim 1, wherein the compound has the formula: 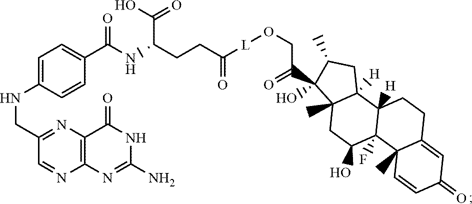 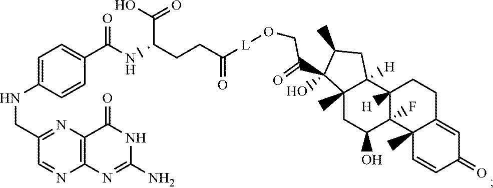 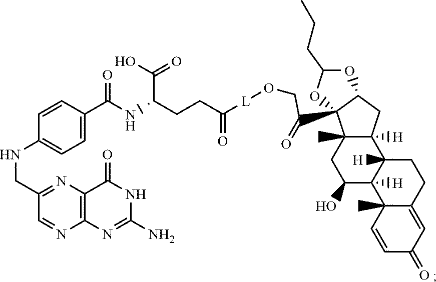 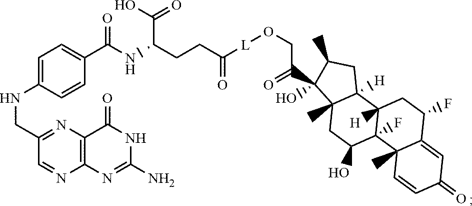 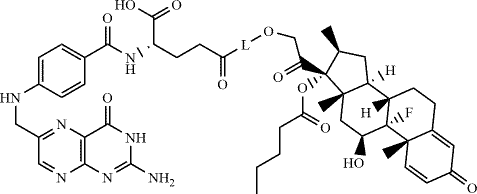 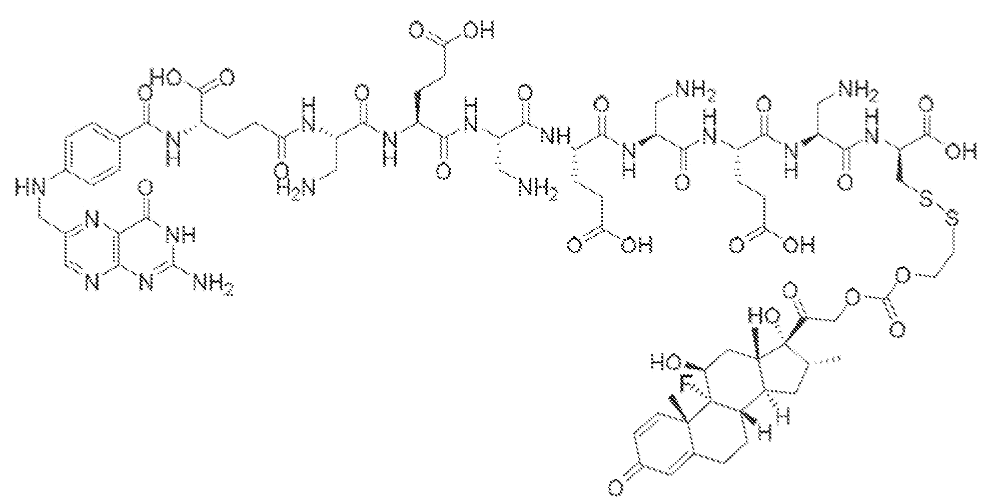 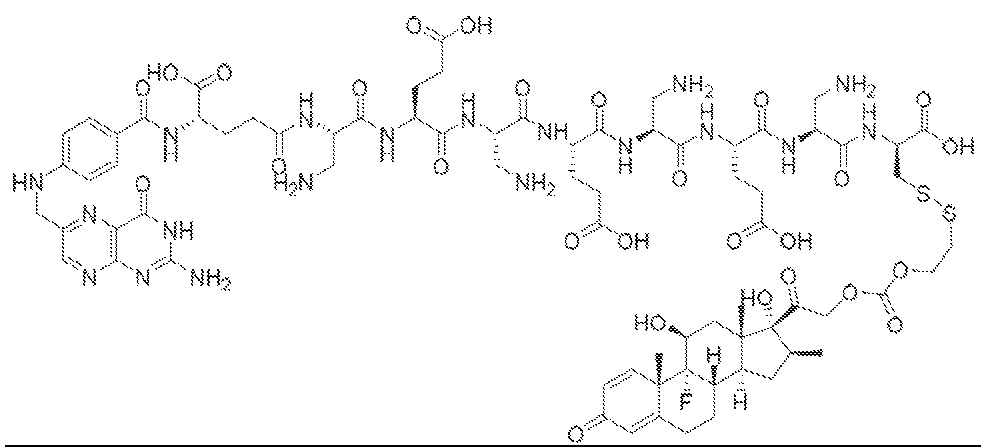 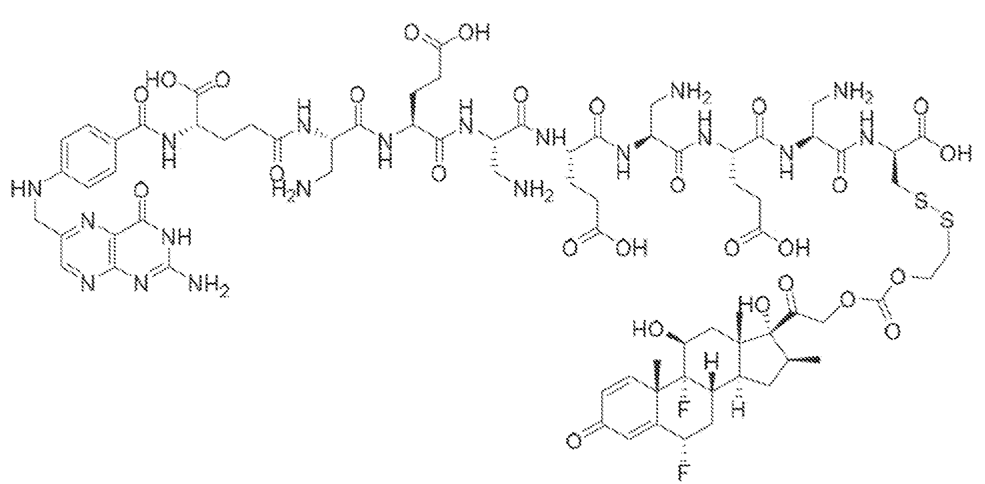 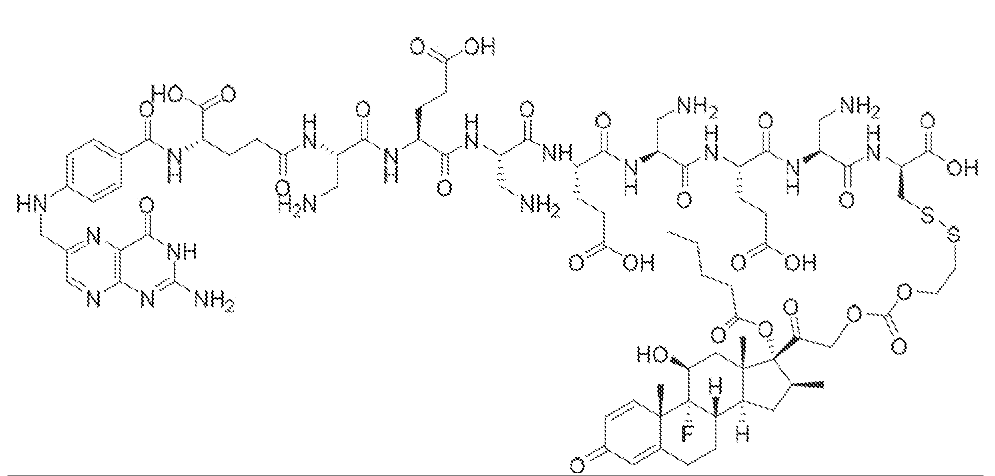 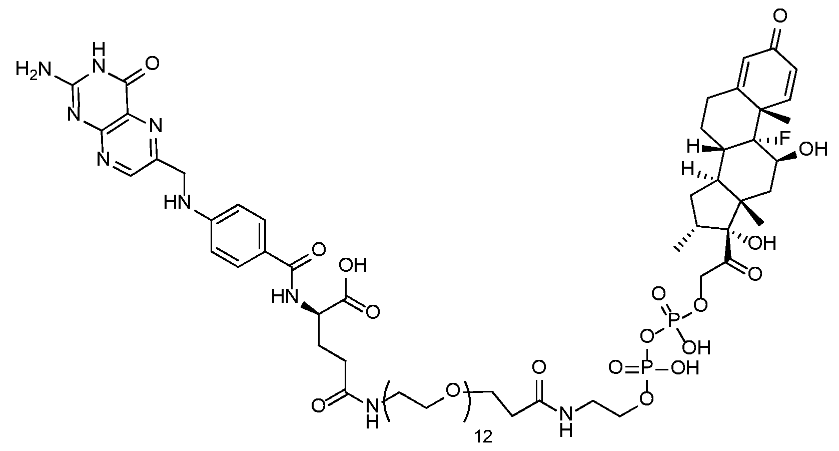 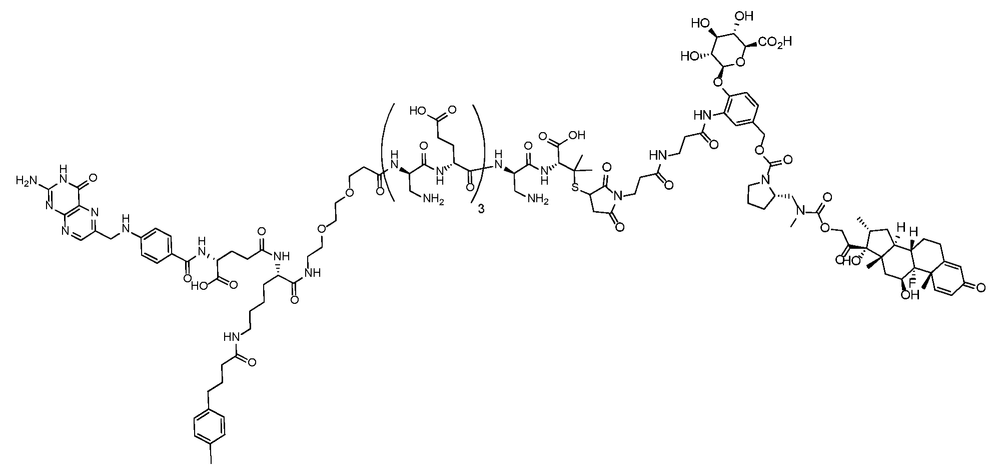 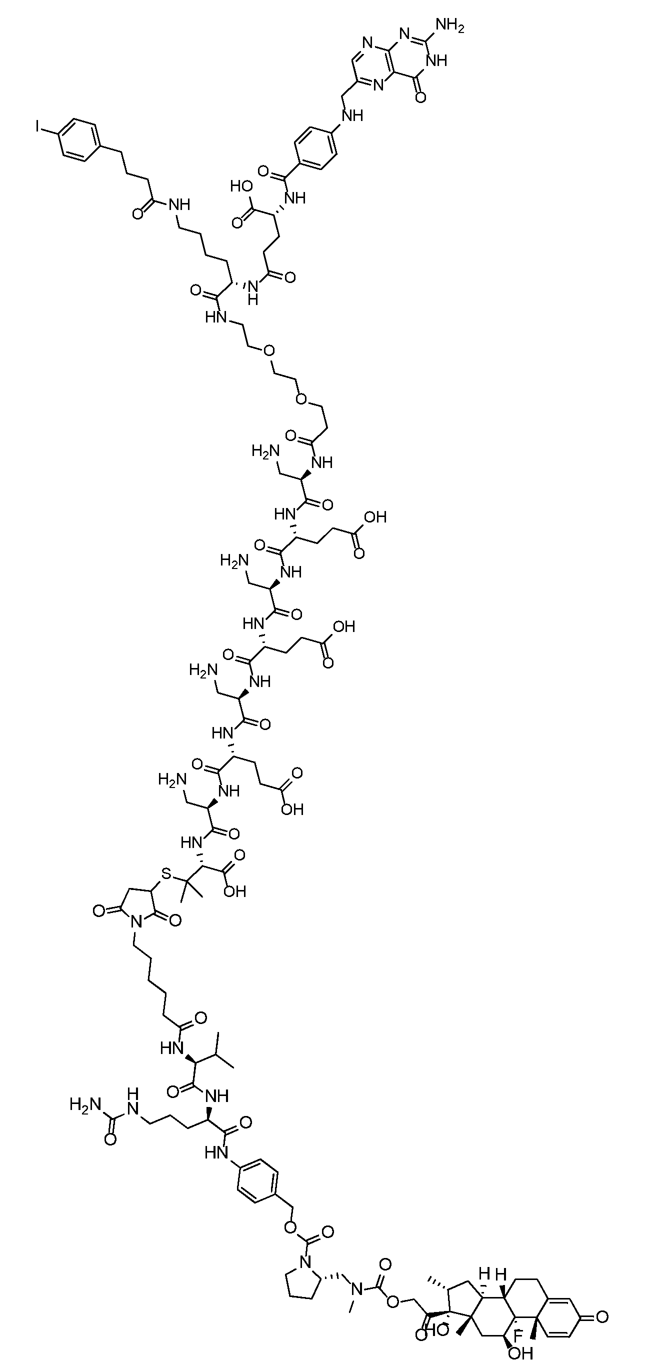
or a pharmaceutically acceptable salt thereof.

48-55. (canceled)

56. A pharmaceutical composition, comprising:
a. a compound of claims 1; and
b. a pharmaceutically acceptable excipient.

57. The pharmaceutical composition of claim 56, wherein a pharmaceutically acceptable excipient is part of a nanoparticle, a liposomal formulation or an exosomal formulation.

58. A method of shifting macrophages from M1 to M2 in a subject in need thereof, the method comprising: administering to the subject a therapeutically effective amount of the compound of claim 1 or a composition of claim 56.

59. A method for treating an inflammatory disorder in a subject in need thereof, the method comprising administering to the subject a therapeutically effective amount of a compound of claim 1 or a composition of claim 56.

60-66. (canceled)

67. The method of claim 59, wherein the inflammatory disorder is Crohn’s disease, lupus, inflammatory bowel disease (IBS), Addison’s disease, Grave’s disease, Sjogren’s syndrome, celiac disease, Hashimoto′ s thyroiditis, myasthenia gravis, autoimmune vasculitis, reactive arthritis, psoriatic arthritis, pernicious anemia, ulcerative colitis, rheumatoid arthritis, type 1 diabetes, multiple sclerosis, or fibrotic disease, graft vs. host disease (GVHD), fatty liver disease, asthma, osteoporosis, sarcoidosis, ischemia-reperfusion injury, prosthesis osteolysis, glomerulonephritis, scleroderma, psoriasis, with autoimmune myocarditis, spinal cord injury, central nervous system, viral infection, influenza, coronavirus infection, cytokine storm syndrome, bone damage, inflammatory brain disease, atherosclerosis, cancer, or a tumor.

68. A method of treating an autoimmune disease in a subject in need thereof, the method comprising administering to the subject a therapeutically effective amount of a compound claim 1 or a composition of claim 56.

69-75. (canceled)

76. A method for treating inflammation in a subject in need thereof, the method comprising administering to the subject a therapeutically effective amount of a compound of claim 1 or a composition of claim 56.

77. The method of claim 76, wherein the inflammation is associated with an autoimmune disease.

78. The method of claim 76, wherein G² is a radical of a steroid selected from the group consisting of dexamethasone, betamethasone, budesonide, fludrocortisone, beclomethasone, fluticasone, mometasone, ciclesonide, cortisone, cortivazol, hydrocortisone, 21-hydroxypregnenolone, meprednisone, dexamethasone, triamcinolone, betamethasone, prednisone, prednisolone, and methylprednisolone.

79-85. (canceled)

86. The method of claim 76, wherein the inflammation is associated with an immune disorder or disease, with a neuromuscular disorder or disease, with a hormonal disorder or disease, with a gastrointestinal disorder or disease, with a connective tissue disease or disorder, with a liver disease or disorder, with a musculoskeletal disease or disorder, with a blood disease of disorder, with a metabolic disease or disorder, with a metabolic disease or disorder, with an endocrine disease or disorder, with an infection, with a neurological disease or disorder, with a renal disease or disorder, with a pulmonary disease or disorder, or with a tissue disease or disorder,.

87-101. (canceled)

102. The method of claim 76, wherein the inflammation is associated with Crohn’s disease, lupus, inflammatory bowel disease (IBS), Addison’s disease, Grave’s disease, Sjogren’s syndrome, celiac disease, Hashimoto’s thyroiditis, myasthenia gravis, autoimmune vasculitis, reactive arthritis, psoriatic arthritis, pernicious anemia, ulcerative colitis, rheumatoid arthritis, type 1 diabetes, multiple sclerosis, or fibrotic disease, graft vs. host disease (GVHD), fatty liver disease, asthma, osteoporosis, sarcoidosis, ischemia-reperfusion injury, prosthesis osteolysis, glomerulonephritis, scleroderma, psoriasis, with autoimmune myocarditis, spinal cord injury, central nervous system, viral infection, influenza, coronavirus infection, cytokine storm syndrome, bone damage, inflammatory brain disease, atherosclerosis, cancer, or a tumor.

103. The method of claim 76, wherein the:
inflammation is associated with Crohn’s disease, and G² is a radical of a steroid selected from the group consisting of dexamethasone, hydrocortisone, budesonide, betamethasone, prednisone, prednisolone, and methylprednisolone;
inflammation associated with lupus, and G² is a radical of a steroid selected from the group consisting of dexamethasone, hydrocortisone, betamethasone, budesonide, prednisone, prednisolone, and methylprednisolone;
inflammation associated with inflammatory bowel disease, and wherein G² is a radical of a steroid selected from the group consisting of dexamethasone, betamethasone, hydrocortisone, budesonide, prednisone, prednisolone, and methylprednisolone;
inflammation associated with Addison’s disease, and G² is a radical of a steroid selected from the group consisting of dexamethasone, betamethasone, hydrocortisone, fludrocortisone, prednisone, prednisolone, and methylprednisolone;
inflammation associated with Grave’s disease, and G² is a radical of a steroid selected from the group consisting of dexamethasone, betamethasone, budesonide, prednisone, prednisolone, and methylprednisolone;
inflammation associated with Sjogren’s syndrome, and G² is a radical of a steroid selected from the group consisting of dexamethasone, betamethasone, budesonide, prednisone, prednisolone, and methylprednisolone;
inflammation associated with celiac disease, and G² is a radical of a steroid selected from the group consisting of dexamethasone, betamethasone, budesonide, prednisone, prednisolone, and methylprednisolone;
inflammation associated with Hashimoto’s thyroiditis, and wherein G² is a radical of a steroid selected from the group consisting of dexamethasone, betamethasone, budesonide, prednisone, prednisolone, and methylprednisolone;
inflammation associated with myasthenia gravis, and G² is a radical of a steroid selected from the group consisting of dexamethasone, betamethasone, budesonide, prednisone, prednisolone, and methylprednisolone;
inflammation associated with autoimmune vasculitis, and G² is a radical of a steroid selected from the group consisting of dexamethasone, betamethasone, budesonide, prednisone, prednisolone, and methylprednisolone;
inflammation associated with reactive arthritis, and G² is a radical of a steroid selected from the group consisting of cortisone, hydrocortisone, dexamethasone, betamethasone, budesonide, prednisone, prednisolone, and methylprednisolone;
inflammation associated with psoriatic arthritis, and G² is a radical of a steroid selected from the group consisting of cortisone, hydrocortisone, dexamethasone, betamethasone, budesonide, prednisone, prednisolone, and methylprednisolone;
inflammation associated with pernicious anemia, and G² is a radical of a steroid selected from the group consisting of dexamethasone, betamethasone, prednisone, prednisolone, and methylprednisolone;
inflammation associated with ulcerative colitis, and G² is a radical of a steroid selected from the group consisting of cortisone, hydrocortisone, triamcinolone, beclomethasone, betamethasone, dexamethasone, budesonide, prednisone, prednisolone, and methylprednisolone;
inflammation associated with rheumatoid arthritis, and G² is a radical of a steroid selected from the group consisting of cortisone, hydrocortisone, triamcinolone, beclomethasone, betamethasone, dexamethasone, budesonide, prednisone, prednisolone, and methylprednisolone;
inflammation associated with type 1 diabetes, and G² is a radical of a steroid selected from the group consisting of dexamethasone, betamethasone, prednisone, prednisolone, and methylprednisolone;
inflammation associated with multiple sclerosis, and G² is a radical of a steroid selected from the group consisting of dexamethasone, betamethasone, prednisone, prednisolone, and methylprednisolone;
inflammation associated with asthma, and G² is a radical of a steroid selected from the group consisting of triamcinolone, fluticasone, budesonide, mometasone, beclomethasone, ciclesonide, dexamethasone, betamethasone, budesonide, prednisone, prednisolone, and methylprednisolone;
inflammation associated with osteoporosis, and G² is a radical of a steroid selected from the group consisting of dexamethasone, betamethasone, prednisone, prednisolone, and methylprednisolone;
inflammation associated with sarcoidosis, and G² is a radical of a steroid selected from the group consisting of dexamethasone, betamethasone, prednisone, prednisolone, and methylprednisolone;
inflammation associated with glomerulonephritis, and G² is a radical of a steroid selected from the group consisting of dexamethasone, betamethasone, budesonide, prednisone, prednisolone, and methylprednisolone;
inflammation associated with autoimmune myocarditis, and G² is a radical of a steroid selected from the group consisting of dexamethasone, betamethasone, budesonide, prednisone, prednisolone, and methylprednisolone;
inflammation associated with a fibrotic disease, and G² is a radical of a steroid selected from the group consisting of dexamethasone, betamethasone, budesonide, and prednisone;
inflammation associated with graft vs. host disease (GVHD), and G² is a radical of a steroid selected from the group consisting of dexamethasone, betamethasone, budesonide, prednisone, prednisolone, and methylprednisolone;
inflammation associated with fatty liver disease, and G² is a radical of a steroid selected from the group consisting of dexamethasone, betamethasone, budesonide, prednisone, prednisolone, and methylprednisolone;
inflammation associated with ischemia-reperfusion injury, and G² is a radical of a steroid selected from the group consisting of dexamethasone, betamethasone, budesonide, prednisone, prednisolone, and methylprednisolone;
inflammation associated with prosthesis osteolysis, and G² is a radical of a steroid selected from the group consisting of dexamethasone, betamethasone, budesonide, prednisone, prednisolone, and methylprednisolone;
inflammation associated with scleroderma, and G² is a radical of a steroid selected from the group consisting of dexamethasone, budesonide, betamethasone, triamcinolone, prednisone, prednisolone, and methylprednisolone;
inflammation associated with psoriasis, and G² is a radical of a steroid selected from the group consisting of budesonide, betamethasone, triamcinolone, prednisone, prednisolone, methylprednisolone, hydrocortisone, dexamethasone, hydrocortisone-17-valerate, diflorasone, meprednisone, halobetacol, tixocortol, amcinonide, desonide, fluocinolone acetonide, fluocinonide, halcinonide, beclomethasone, and halometasone;
inflammation associated with spinal cord injury, and G² is a radical of a steroid selected from the group consisting of dexamethasone, betamethasone, budesonide, prednisone, prednisolone, and methylprednisolone;
inflammation of the central nervous system, and G² is a radical of a steroid selected from the group consisting of dexamethasone, betamethasone, budesonide, prednisone, prednisolone, and methylprednisolone;
inflammation associated with a viral infection, and G² is a radical of a steroid selected from the group consisting of hydrocortisone, dexamethasone, betamethasone, budesonide, prednisone, prednisolone, and methylprednisolone;
inflammation associated with influenza, and G² is a radical of a steroid selected from the group consisting of hydrocortisone, dexamethasone, betamethasone, budesonide, prednisone, prednisolone, and methylprednisolone;
inflammation associated with SARS-CoV-2 (COVID-19), and G² is a radical of a steroid selected from the group consisting of hydrocortisone, dexamethasone, betamethasone, budesonide, and prednisone, prednisolone, and methylprednisolone;
inflammation associated with cytokine storm syndrome, and G² is a radical of a steroid selected from the group consisting of hydrocortisone, dexamethasone, betamethasone, budesonide, prednisone, prednisolone, and methylprednisolone;
inflammation associated with damage to bone, and G² is a radical of a steroid selected from the group consisting of cortisone, cortivazol, hydrocortisone, 21-hydroxypregnenolone, meprednisone, dexamethasone, triamcinolone, betamethasone, budesonide, prednisone, prednisolone, and methylprednisolone;
inflammation associated with inflammatory brain disease, and G² is a radical of a steroid selected from the group consisting of dexamethasone, betamethasone, budesonide, prednisone, prednisolone, and methylprednisolone;
inflammation associated with atherosclerosis, and G² is a radical of a steroid selected from the group consisting of fluticasone, budesonide, beclomethasone, ciclesonide, dexamethasone, betamethasone, prednisone, prednisolone, and methylprednisolone; inflammation associated with a tumor, and wherein G² is a radical of a steroid selected from the group consisting of cortisone, hydrocortisone, clobetasol, dexamethasone, betamethasone, budesonide, meprednisone, prednisone, prednisolone, and methylprednisolone;and
inflammation associated with cancer, and G² is a radical of a steroid selected from the group consisting of cortisone, hydrocortisone, clobetasol, dexamethasone, betamethasone, budesonide, meprednisone, prednisone, prednisolone, and methylprednisolone.

104-142. (canceled)

143. A method for treating a disease or disorder that involves polarizing macrophages from M1 to M2 in a subject in need thereof, the method comprising administering to the subject a therapeutically effective amount of a compound of claim 1 or a pharmaceutical composition of claim 56.
